(19) United States
(12) Patent Application Publication
(10) Pub. No.: US 2010/0310501 A1
(43) Pub. Date: Dec. 9, 2010

(54) Methods for Improving Immune Function and Methods for Prevention or Treatment of Disease in a Mammalian Subject

(76) Inventors: Onur Boyman, Lausanne (CH); Charles D. Surh, Poway, CA (US); Jonathan Sprent, Potts Point (AU); Mark Rubinstein, La Jolla, CA (US); Marek Kovar, Prague (CZ)

Correspondence Address: FENWICK & WEST LLP, SILICON VALLEY CENTER, 801 CALIFORNIA STREET, MOUNTAIN VIEW, CA, 94041, US

(21) Appl. No.: 12/279,264

(22) PCT Filed: Feb. 16, 2007
(86) PCT No.: PCT/US07/62361

§ 371 Date: May 1, 2009

Related U.S. Application Data
(60) Provisional application No. 60/773,924, filed on Feb. 16, 2006.

(51) Int. Cl. A61K 38/20; C07K 16/24; A61K 38/19; A61K 38/21; A61P 17/06; A61P 19/02; A61P 3/10; A61P 29/00; A61P 11/06; A61P 37/08; A61P 37/00; A61P 35/02; A61P 35/00; A61P 37/04

(52) U.S. Cl. 424/85.2; 530/389.2; 514/1.1; 424/85.5; 424/85.6; 424/85.7

(57) ABSTRACT

A method for increasing a biological activity of a cytokine or lymphokine and a method of treating a neoplastic disease, autoimmune disease, or infectious disease, and a method for expanding a hematopoietic cell population, is provided by administering an antibody capable of binding a cytokine or by administering a cytokine complexed with an antibody or by administering a cytokine complexed with a cytokine receptor to a mammalian subject in need thereof.

### CROSS REFERENCE TO RELATED APPLICATION

This application claims the benefit of U.S. Provisional Application No. 60/773,924, filed Feb. 16, 2006, which is hereby incorporated by reference in its entirety.

### STATEMENT OF GOVERNMENT SUPPORT

This invention was made with government support by The National Institutes of Health Grant No. AI24187, AI46710, CA38355, AI45809, AI007244, AG001743, and AG20189. The Government has certain rights in this invention.

### FIELD

The invention relates to a method for increasing a biological activity of a cytokine or lymphokine by administering an antibody capable of binding a cytokine or by administering a cytokine complexed with an antibody or administering a cytokine complexed with a cytokine receptor to a mammalian subject in need thereof. The invention further relates to a method of treating a neoplastic disease, autoimmune disease, or infectious disease, or a method for expanding a hematopoietic cell population, by administering a cytokine complexed with an antibody or administering a cytokine complexed with a cytokine receptor to a mammalian subject in need thereof.

### BACKGROUND

Contact with certain cytokines, notably IL-2, IL-7 and IL-15, maintains the survival of naïve and memory T cells. Smith, Science 240: 1169, 1988; Waldmann, Annu Rev Biochem 58: 875, 1989; Ku et al., Science 288: 675, 2000; Sprent et al., Annu Rev Immunol 20: 551, 2002; Schluns et al., Nat Rev Immunol 3: 269, 2003. Responsiveness to IL-2 and IL-15 is controlled largely by a shared dimeric receptor comprised of a β-chain (CD122) and a common γ-chain (γ_{c}). Waldmann, Annu Rev Biochem 58: 875, 1989; Takeshita et al., Science 257: 379, 1992; Nakamura et al., Nature 369: 330, 1994. CD122 expression is especially high on “memory” CD8⁺ cells primed against defined antigens and also on a naturally-occurring population of CD8⁺ cells with a similar phenotype. These latter CD122^{high(hi)} memory-phenotype (MP) CD8⁺ cells proliferate in response to IL-2 or IL-15 in vitro, and IL-15 also controls their survival and intermittent proliferation (turnover) in vivo. Smith, Science 240: 1169, 1988; Zhang et al., Immunity 8: 591, 1998; Sprent et al., Annu Rev Immunol 20: 551, 2002. Responsiveness to IL-7 is controlled by a dimeric receptor comprised of an α-chain (CD127) and a γ_{c}-chain; IL-7 receptor is highly expressed on both naïve and memory T cells. Goodwin et al., Cell 60:941, 1990; Sudo et al., Proc. Natl. Acad. Sci. 90:9125, 1993; Tan et al. J. Exp. Med. 195:1523, 2002.

IL-2 is also vital for the survival of CD4⁺T regs in vivo. Malek et al., Nat Rev Immunol 4: 665, 2004; Fontenot et al., Nat Immunol 6: 331, 2005. These latter cells are characterized by strong constitutive expression of IL-2Rα (CD25), which enables the cells to express a high-affinity trimeric αβγ_{c} receptor (IL-2Rαβγ_{c}) and thereby utilize low levels of IL-2. Reflecting their dependency on IL-2, CD4⁺T regs disappear after injection of an anti-IL-2 monoclonal antibody (IL-2 mAb). Murakami et al., Proc Natl Acad Sci USA 99: 8832, 2002; Setoguchi et al., J Exp Med 201: 723, 2005.

IL-15 is normally presented in vivo as a cell-associated cytokine bound to IL-15Rα. IL-15Rα plays a mandatory role in presenting endogenous IL-15. Thus, like IL-15^{-/-} mice (Kennedy et al., J Exp Med 191:771-80, 2000), IL-15Rα^{-/-} mice lack CD122hi CD8+ cells and NK cells (Lodolce et al., Immunity 9:669-76, 1998), presumably because the IL-15 synthesized in IL-15R^{-/-} mice fails to leave the cytoplasm. Nevertheless, IL-2Rβγ_{c}⁺ cells can proliferate in response to a soluble recombinant form of IL-15 in the absence of IL-15Rα (Lodolce et al., J Exp Med 194:1187-94, 2001). Moreover, under certain conditions, IL-15Rα can be inhibitory. Thus, injecting mice with a soluble (s) recombinant form of IL-15Rα is reported to suppress NK cell proliferation (Nguyen et al., J Immunol 169:4279-87, 2002) and certain T-dependent immune responses in vivo (Ruckert et al., Eur J Immunol 33:3493-3503, 2003; Ruckert et al., J Immunol 174:5507-15, 2005; Wei et al., J Immunol 167:577-82, 2001; Ruchatz et al., J Immunol 160:5654-5660, 1998), and adding sIL-15Rα in vitro can block the response of cell lines to IL-15 (Ruckert et al., Eur J Immunol 33:3493-3503, 2003; Ruckert et al., J Immunol 174:5507-15, 2005; Wei et al., J Immunol 167:577-82, 2001; Ruchatz et al., J Immunol 160:5654-5660, 1998; Budagian et al., J Biol Chem 279:40368-75, 2004; Mortier et al., J Immunol 173:1681-1688, 2004; Eisenman et al., Cytokine 20:121-29, 2002). Despite these findings, there are other reports that sIL-15Rα (Giron-Michel et al., Blood 106:2302-10, 2005), and also a soluble sushi domain of IL-15Rα (Mortier et al., J Biol Chem, 2005, E-pub ahead of print), can enhance IL-15 responses of human cell lines. A need exists in the art for a therapy to improve immune function in a mammalian subject and for improved methods for treating disease such as autoimmune disease, neoplastic disease, or infectious disease by administration of a cytokine to the mammalian subject.

### SUMMARY

The present invention generally relates to methods for treating disease by administering to a mammalian subject in need thereof, a composition comprising an antibody capable of binding a cytokine or a composition comprising a cytokine and a receptor to the cytokine. The present invention further relates to methods for treating disease by complexing the antibody with the cytokine prior to the administration, and administering the cytokine antibody complex to the mammalian subject. The present invention further relates to methods for treating disease by complexing the cytokine with the cytokine receptor prior to the administration, and administering the cytokine/cytokine receptor complex to the mammalian subject. A method for improving immune function in a mammalian subject is provided by administering a composition comprising an antibody capable of binding a cytokine or a cytokine complexed with an antibody thereby increasing a biological activity of the cytokine in the mammalian subject. A method for improving immune function in a mammalian subject is provided by administering a composition comprising a cytokine complexed with a cytokine receptor thereby increasing a biological activity of the cytokine in the mammalian subject. The disease state includes, but is not limited to neoplastic disease, autoimmune disease, infectious disease, or hematopoietic cell depletion resulting from irradiation or cytotoxic drug treatment, or primary or secondary immunodeficiency, or aging. The cytokine antibody complex can be a cytokine and an antibody, e.g., a monoclonal antibody, bound to the cytokine. The cytokine/cytokine receptor complex can be, for example, an interleukin 15/interleukin-15 receptor α complex. The method for increasing a biological activity of a cytokine by administering a cytokine complexed with an antibody or a cytokine/cytokine receptor complex can occur as a result of expansion of hematopoietic cells or a subpopulation of T cells, e.g., expansion of CD8⁺ T cells and CD4⁺ T regulatory cells, or expansion of CD8⁺T cells, or expansion of CD4⁺T regulatory cells while blocking expansion of CD8⁺ T cells, or expansion of naïve T cells (both CD4⁺ T cells and CD8 T cells) or memory T cells, or a combination thereof.

A method for improving immune function in a mammalian subject is provided comprising administering to the mammalian subject an antibody capable of binding a cytokine thereby increasing a biological activity of the cytokine in the mammalian subject. A method for improving immune function in a mammalian subject is provided comprising administering to the mammalian subject a cytokine bound to a cytokine receptor thereby increasing a biological activity of the cytokine in the mammalian subject. The method for improving immune function can result from increasing presentation of the cytokine to a target cell in the mammalian subject. The method further comprises complexing the antibody with the cytokine prior to said administration, and administering the cytokine antibody complex to the mammalian subject. In one aspect, a monoclonal antibody comprising an Fc portion binds to the cytokine. The cytokine includes, but is not limited to, IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ. The cytokine receptor can be a natural receptor of the cytokine, for example, interleukin-15 receptor α capable of binding interleukin-15.

Many variants of the method, are envisioned. For example, in one variant, increasing the biological activity of the cytokine expands a population of hematopoietic cells. In a further variant, increasing the biological activity of the cytokine expands a population of T cells, B cells, or NK cells, or a combination thereof. In a further variant, increasing the biological activity of the cytokine expands CD8⁺ T cells and CD4⁺ T regulatory cells. In another aspect, increasing the biological activity of the cytokine expands CD8⁺T cells. In a further aspect, increasing the biological activity of the cytokine expands CD4⁺T cells. In a further aspect, increasing the biological activity of the cytokine expands CD4⁺ T regulatory cells and blocks expansion of CD8⁺ T cells. In another aspect, increasing the biological activity of the cytokine expands naïve T cells or memory T cells, or a combination thereof. In a variant of the method, increasing the biological activity of type I interferons or type II interferons on a non-hematopoietic cell improves immune function in the mammalian subject. In another aspect, increasing the biological activity of the cytokine expands the cell population ex vivo. In a further aspect, increasing the biological activity of the cytokine expands the cell population in vivo.

A method for improving immune function in a mammalian subject is provided comprising administering to the mammalian subject an antibody capable of binding cytokine, thereby increasing a biological activity of cytokine in the mammalian subject. In one aspect, the cytokine can be interleukin-2. In a further aspect, the cytokine can be interleukin-7. The method for improving immune function can result from increasing presentation of cytokine to a target cell in the mammalian subject. The method further comprises complexing the antibody with cytokine prior to the administration, and administering the cytokine antibody complex to the mammalian subject. The method further comprises complexing the cytokine with its cytokine receptor prior to the administration, and administering the cytokine/cytokine receptor complex to the mammalian subject. In one aspect, the monoclonal antibody or the cytokine receptor comprising an Fc portion binds to the cytokine. In a further aspect, the mammalian subject has a weakened immune system due to advanced age of the mammalian subject. In one aspect of the method, increasing presentation of the cytokine to a target cell to improve immune function expands naïve T cells or memory T cells, or a combination thereof. The method can provide the therapeutic effect which reduces or eliminates neoplastic disease, autoimmune disease, or infectious disease in the mammalian subject, or prevents its occurrence or recurrence. The method can provide the therapeutic effect which expands a hematopoietic cell population or improves hematopoietic cell recovery from cell depletion resulting from irradiation or cytotoxic drug treatment, or primary or secondary immunodeficiency in the mammalian subject, or aging.

A method for preventing or treating autoimmune disease in a mammalian subject is provided comprising administering an antibody capable of binding a cytokine to the mammalian subject in an amount effective to reduce or eliminate the autoimmune disease or to prevent its occurrence or recurrence. The method further comprises complexing the antibody with the cytokine prior to said administration, and administering the cytokine antibody complex to the mammalian subject. The method further comprises complexing the cytokine with its cytokine receptor prior to the administration, and administering the cytokine/cytokine receptor complex to the mammalian subject. In one aspect, a monoclonal antibody comprising an Fc portion binds to the cytokine. The cytokine includes, but is not limited to, IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ. The autoimmune disease includes, but is not limited to, rheumatoid arthritis, multiple sclerosis, diabetes, inflammatory bowel disease, psoriasis, systemic lupus erythematosus, allergic disease, or asthma.

Many variants of the method, are envisioned. In a further aspect the method comprises increasing a biological activity of the cytokine. For example, in one variant, increasing the biological activity of the cytokine expands a population of hematopoietic cells. In a further variant, increasing the biological activity of the cytokine expands a population of T cells, B cells, or NK cells, or a combination thereof. In a further variant, increasing the biological activity of the cytokine expands CD8⁺T cells and CD4⁺T regulatory cells. In another aspect, increasing the biological activity of the cytokine expands CD8⁺ T cells. In a further aspect, increasing the biological activity of the cytokine expands CD4⁺ T cells. In a further aspect, increasing the biological activity of the cytokine expands CD4⁺T regulatory cells and blocks expansion of CD8⁺ T cells. In another aspect, increasing the biological activity of the cytokine expands naïve T cells or memory T cells, or a combination thereof. In a variant of the method, increasing the biological activity of type I interferons or type II interferons on a non-hematopoietic cell improves immune function in the mammalian subject. In another aspect, increasing the biological activity of the cytokine expands the cell population ex vivo. In a further aspect, increasing the biological activity of the cytokine expands the cell population in vivo.

A method for preventing or treating neoplastic disease in a mammalian subject is provided comprising administering an antibody capable of binding a cytokine to the mammalian subject in an amount effective to reduce or eliminate the neoplastic disease or to prevent its occurrence or recurrence. The neoplastic disease includes, but is not limited to, cancer, solid tumor, sarcoma, melanoma, carcinoma, leukemia, or lymphoma. The method further comprises complexing the antibody with the cytokine prior to said administration, and administering the cytokine antibody complex to the mammalian subject. The method further comprises complexing the cytokine with its cytokine receptor prior to the administration, and administering the cytokine/cytokine receptor complex to the mammalian subject. In one aspect, the monoclonal antibody comprising an Fc portion or the cytokine receptor comprising an Fc portion binds to the cytokine. The cytokine includes, but is not limited to, IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ.

The method further provides for increasing a biological activity of the cytokine. Many variants of the method are envisioned. For example, in one variant, increasing the biological activity of the cytokine expands a population of hematopoietic cells. In a further variant, increasing the biological activity of the cytokine expands a population of T cells, B cells, or NK cells, or a combination thereof. In a further variant, increasing the biological activity of the cytokine expands CD8⁺ T cells and CD4⁺ T regulatory cells. In another aspect, increasing the biological activity of the cytokine expands CD8⁺T cells. In a further aspect, increasing the biological activity of the cytokine expands CD4⁺ T cells. In a further aspect, increasing the biological activity of the cytokine expands CD4⁺ T regulatory cells and blocks expansion of CD8⁺T cells. In another aspect, increasing the biological activity of the cytokine expands naïve T cells or memory T cells, or a combination thereof. In a variant of the method, increasing the biological activity of type I interferons or type II interferons on a non-hematopoietic cell improves immune function in the mammalian subject. In another aspect, increasing the biological activity of the cytokine expands the cell population ex vivo. In a further aspect, increasing the biological activity of the cytokine expands the cell population in vivo.

A method for improving immune function in a mammalian subject is provided which comprises administering to the mammalian subject an interleukin-15 and an interleukin-15 receptor α, and thereby increasing a biological activity of the interleukin-15 in the mammalian subject. The method can further comprise increasing presentation of the interleukin-15 to a target cell to improve immune function in the mammalian subject. The method can further comprise complexing the interleukin-15 with the interleukin-15 receptor α prior to said administration, and administering the interleukin-15/interleukin-15 receptor α complex to the mammalian subject. In one aspect of the method, increasing presentation of the interleukin-15 to a target cell to improve immune function expands naïve T cells or memory T cells, or a combination thereof. The increased biological activity can have a therapeutic effect to reduce or eliminate neoplastic disease or infectious disease in the mammalian subject, or prevents its occurrence or recurrence. The increased biological activity can have a therapeutic effect to expand a hematopoietic cell population or improve hematopoietic cell recovery from cell depletion resulting from irradiation or cytotoxic drug treatment, or from primary or secondary immunodeficiency in the mammalian subject, or from aging in the mammalian subject. In a further aspect, the mammalian subject has a weakened immune system due to advanced age of the mammalian subject.

A method for expanding a hematopoietic cell population in a mammalian subject is provided comprising administering an antibody capable of binding a cytokine to the mammalian subject, thereby providing a therapeutic effect of the expanded hematopoietic cell population in the mammalian subject. The method further comprises complexing the antibody with the cytokine prior to said administration, and administering the cytokine antibody complex to the mammalian subject. The method further comprises complexing the cytokine with its cytokine receptor prior to the administration, and administering the cytokine/cytokine receptor complex to the mammalian subject. In one aspect, a monoclonal antibody comprising an Fc portion or a cytokine receptor comprising an Fc portion binds to the cytokine. The cytokine includes, but is not limited to, IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ.

The method further provides for increasing a biological activity of the cytokine. Many variants of the method are envisioned. For example, in one variant, increasing the biological activity of the cytokine expands a population of hematopoietic cells. In a further variant, increasing the biological activity of the cytokine expands a population of T cells, B cells, or NK cells, or a combination thereof. In a further variant, increasing the biological activity of the cytokine expands CD8⁺T cells and CD4⁺T regulatory cells. In another aspect, increasing the biological activity of the cytokine expands CD8⁺ T cells. In a further aspect, increasing the biological activity of the cytokine expands CD4⁺ T cells. In a further aspect, increasing the biological activity of the cytokine expands CD4⁺T regulatory cells and blocks expansion of CD8⁺ T cells. In another aspect, increasing the biological activity of the cytokine expands naïve T cells or memory T cells, or a combination thereof. In a further aspect, increasing the biological activity of the cytokine can expand the NK cell population or can expand the B cell population. In a further aspect, a therapeutic effect of a cytokine antibody complex can improve hematopoietic cell recovery from hematopoietic cell depletion resulting from irradiation or cytotoxic drug treatment, or primary or secondary immunodeficiency in the mammalian subject. In another aspect, increasing the biological activity of the cytokine expands the cell population ex vivo. In a further aspect, increasing the biological activity of the cytokine expands the cell population in vivo.

A method for preventing or treating infectious disease in a mammalian subject is provided comprising administering an antibody capable of binding a cytokine to the mammalian subject in an amount effective to reduce or eliminate the infectious disease or to prevent its occurrence or recurrence. The method further comprises complexing the antibody with the cytokine prior to said administration, and administering the cytokine antibody complex to the mammalian subject. The method further comprises complexing the cytokine with its cytokine receptor prior to the administration, and administering the cytokine/cytokine receptor complex to the mammalian subject. In a further aspect, the antibody or the cytokine complexed with an antibody, or cytokine complexed with its receptor is administered with a vaccine to increase an immune response and to enhance vaccine efficacy. In one aspect, a monoclonal antibody comprising an Fc portion binds to the cytokine. The cytokine includes, but is not limited to, IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ.

The method further provides for increasing a biological activity of the cytokine. Many variants of the method are envisioned. For example, in one variant, increasing the biological activity of the cytokine expands a population of hematopoietic cells. In a further variant, increasing the biological activity of the cytokine expands a population of T cells, B cells, or NK cells, or a combination thereof. In a further variant, increasing the biological activity of the cytokine expands CD8⁺ T cells and CD4⁺ T regulatory cells. In another aspect, increasing the biological activity of the cytokine expands CD8⁺ T cells. In a further aspect, increasing the biological activity of the cytokine expands CD4⁺T cells. In a further aspect, increasing the biological activity of the cytokine expands CD4⁺ T regulatory cells and blocks expansion of CD8⁺ T cells. In another aspect, increasing the biological activity of the cytokine expands naïve T cells or memory T cells, or a combination thereof. In a variant of the method, increasing the biological activity of type I interferons or type II interferons on a non-hematopoietic cell improves immune function in the mammalian subject. In a further aspect, increasing the biological activity of the cytokine expands the natural killer cell population or expands the B cell population.

### BRIEF DESCRIPTION OF THE DRAWINGS

FIGS. 1A and 1B show stimulation of memory phenotype (MP) CD8⁺ cells in vivo by IL-2 or IL-2 monoclonal antibody.

FIG. 2 shows proliferation of MP CD8⁺ cells in vivo in response to IL-2 or IL-2 monoclonal antibody.

FIGS. 3A, 3B, and 3C show marked selective expansion of MP and antigen (Ag)-specific memory CD8⁺T cells in vivo by a combination of IL-2 and IL-2 monoclonal antibody.

FIGS. 4A and 4B shows proliferation of CD8⁺ T cells to IL-2/IL-2 monoclonal antibody complexes is largely confined to CD122^{hi} MP cells and is IL-15-independent.

FIGS. 5A and 5B show proliferation of MP CD8⁺ cells to IL-2/IL-2 monoclonal antibody complexes in vivo does not require CD25.

FIGS. 6A, 6B, 6C, 6D, and 6E show selective stimulation of T cell subsets by different IL-2/IL-2 monoclonal antibody complexes.

FIGS. 7A, 7B, 7C, and 7D show requirements for stimulating MP CD8⁺ cells with IL-2/IL-2 monoclonal antibody complexes in vivo.

FIGS. 8A, 8B, 8C, and 8D show features of T cell stimulation by cytokine/monoclonal antibody complexes.

FIGS. 9A and 9B show JES6-5 and S4B6 IL-2 monoclonal antibody bind to similar sites on IL-2 which are distinct from the binding site of JES6-1.

FIGS. 10A and 10B show effects of IL-2/IL-2 monoclonal antibody complexes in vitro.

FIGS. 11A and 11B show injecting a mixture of S4B6 and JES6-1 IL-2 monoclonal antibodies blocks proliferation of both MP CD8⁺ cells and CD4⁺ CD25⁺ cells.

FIGS. 12A and 12B show F(ab′)₂ fragments of IL-2 monoclonal antibody are less efficient than whole IL-2 monoclonal antibody.

FIG. 13 shows IL-2/IL-2 monoclonal antibody complexes are significantly more potent than IL-2-antibody fusion proteins in inducing proliferation of MP CD8⁺ cells.

FIGS. 14A and 14B show IL-7/IL-7 monoclonal antibody complex can efficiently induce T cell development in the thymus.

FIGS. 15A, 15B, and 15C show IL-7/IL-7 monoclonal antibody complex can efficiently induce homeostatic expansion of naïve T cells.

FIG. 16 shows IL-7/IL-7 monoclonal antibody complex can drive expansion of both naïve and memory T cells.

FIG. 17 shows Fc portion of anti-IL-7 monoclonal antibody is required to for proliferative activity of IL-7/IL-7 monoclonal antibody complex.

FIGS. 18A and 18B show that aging is associated with a severe decline in the ability to support homeostatic proliferation of naïve T cells and this can be restored using IL-7/IL-7 monoclonal antibody complexes.

FIGS. 19A, 19B, 19C, 19D, and 19E show soluble IL-15Rα augments IL-15-mediated lymphocyte proliferation in vitro.

FIGS. 20A, 20B, 20C, and 20D show soluble IL-15Rα augments IL-15-mediated donor lymphocyte proliferation in vivo.

FIGS. 21A, 21B, 21C, and 21D show soluble IL-15Rα augments IL-15-mediated host lymphocyte proliferation.

FIG. 22 shows IL-15Rα-Fc are better than IL-15Rα in augmenting IL-15 under in vivo conditions.

FIGS. 23A and 23B show proliferation to IL-15 immobilized by IL-15Rα cannot be blocked by soluble IL-15Rα-Fc.

FIGS. 24A and 24B show soluble IL-2Rα inhibits IL-2-mediated proliferation.

FIGS. 25A and 25B show survival of IL-15 in vitro.

FIG. 26 shows human sIL-15Rα-Fc enhances the response of mouse MP CD8⁺ cells to both mouse and human IL-15.

FIG. 27 shows stimulation by IL-15/sIL-15-Rα-Fc complexes in IL-15Rα^{-/-} hosts.

FIGS. 28A, 28B, and 28C show blocking effects of sIL-15Rα-Fc for responses to mouse vs human IL-15.

### DETAILED DESCRIPTION

The present invention generally relates to methods for treating disease by administering a composition comprising an antibody capable of binding a cytokine or a composition comprising a cytokine and a receptor to the cytokine to a mammalian subject in need thereof. The present invention further relates to methods for treating disease by complexing the antibody with the cytokine prior to the administration, and administering the cytokine antibody complex to the mammalian subject. The present invention further relates to methods for treating disease by complexing the cytokine with the cytokine receptor prior to the administration, and administering the cytokine/cytokine receptor complex to the mammalian subject. A method for improving immune function in a mammalian subject is provided by administering an antibody capable of binding a cytokine or a cytokine complexed with an antibody thereby increasing a biological activity of the cytokine in the mammalian subject. A method for improving immune function in a mammalian subject is provided by administering a composition comprising a cytokine complexed with a cytokine receptor thereby increasing a biological activity of the cytokine in the mammalian subject. A method of treating a disease state is provided in which the disease state includes, but is not limited to neoplastic disease, autoimmune disease, infectious disease, or lymphocyte depletion resulting from irradiation or cytotoxic drug treatment or primary or secondary immunodeficiency, or aging. The cytokine antibody complex can be a cytokine and an antibody, e.g., a monoclonal antibody, bound to the cytokine. The cytokine/cytokine receptor complex can be, for example, an interleukin 15/interleukin-15 receptor α complex.

The method of improving immune function in a mammalian subject by increasing a biological activity of the cytokine, in one aspect, expands a population of hematopoietic cells in the mammalian subject, e.g., T cells, B cells, or NK cells, or a combination thereof. A method of treating a disease state is provided by administering an antibody capable of binding to interleukin-2 or interleukin-2 complexed with an antibody to a mammalian subject in need thereof. The antibody can be a monoclonal antibody. The increase in biological activity of interleukin-2 is useful for treatment of disease, as described herein. The increased interleukin-2 activity can expand T cell populations, B cell populations, or NK cell populations, or more specifically, expanding populations of CD8⁺T cells, or expanding populations of CD8⁺ T cells and CD4⁺ T regulatory cells, or expanding populations of CD4⁺ T regulatory cells and blocking expansion of CD8⁺ T cells. Hematopoietic cell expansion can be carried out in vivo or ex vivo of the mammalian subject.

A method of treating a disease state is provided by administering an antibody capable of binding to cytokine or cytokine complexed with an antibody to a mammalian subject in need thereof. The antibody can be a monoclonal antibody. The increase in biological activity of cytokine is useful for treatment of disease, as described herein. The increased cytokine activity can expand T cell populations, B cell populations, or NK cell populations, or more specifically, biological activity of the cytokine can expand naïve T cells (both naïve CD4 T cells and CD8⁺ T cells) or memory T cells (both naïve CD4 T cells and CD8 T cells), or a combination thereof.

Interleukin-2 (IL-2), a growth factor for T lymphocytes, can also sometimes be inhibitory. The present invention provides an explanation for the fact that proliferation of CD8⁺ T cells in vivo is increased after injection of monoclonal antibody specific for IL-2 (IL-2 mAb). The present invention demonstrates that IL-2 mAb augments proliferation of CD8⁺ cells by increasing the biological activity of pre-existing IL-2 through formation of immune complexes. When coupled with recombinant IL-2, some IL-2/IL-2 mAb complexes cause massive (>100-fold) expansion of CD8⁺ cells in vivo while others selectively stimulate CD4⁺ T regs. Thus, different cytokine/antibody complexes can be used to selectively boost or inhibit the immune response and are useful for treatment of disease states.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“An amount effective to reduce or eliminate the disease or to prevent its occurrence or recurrence” refers to an amount of a therapeutic compound that improves a patient outcome or survival following treatment for the disease state, e.g., neoplastic disease, autoimmune disease, cell reductive radiation or chemotherapy, or infectious disease, as measured by patient test data, survival data, elevation or suppression of tumor marker levels, reduced susceptibility based upon genetic profile or exposure to environmental factors.

“Lymphocytes” refer to a population of cells in circulation including, but not limited to, T cells, B cells, or natural killer (NK) cells.

“T cell proliferation” refers to growth and expansion of one or more sub-populations of T cells in response to a cellular signal provided by a cytokine or lymphokine. T cell proliferation can occur in vivo or ex vivo. Subpopulations of T cells include, but are not limited to, CD8+ T cells, CD4 T regulatory cells (T reg cells), or natural killer (NK) cells.

“Cytokine antibody complex” or cytokine/cytokine receptor complex” refers to cytokines or lymphokines that are bound to an antibody or to its cytokine receptor either by an electrostatic charge interaction as in an antibody-antigen or ligand-receptor binding interaction. The antibody molecule can be an IgG molecule or a fragment thereof. The antibody fragment can include at least an Fc portion of the antibody molecule.

“Cytokine” refers to the soluble mediators that control many critical interactions among cells of the immune system. Cytokines comprise a diverse group of intercellular signaling peptides and glycoproteins. Most are genetically and structurally similar to each other. Each cytokine is secreted by a particular cell type in response to a variety of stimuli and produces characteristic effects on the growth, mobility, differentiation, and/or function of target cells. Collectively, cytokines regulate not only immune and inflammatory systems, but also are involved in wound healing, hematopoiesis, angiogenesis, and many other processes. It is intended that the term encompass all of the various cytokines, regardless of their structure, and commonly used nomenclature. For example, it is intended that the term encompass “lymphokines” (i.e., cytokines produced by lymphocytes), as well as “monokines” (i.e., cytokines produced by monocytes). Cytokine refers to any one of the numerous factors that exert a variety of effects on cells, for example, inducing growth or proliferation. Non-limiting examples of cytokines which can be used alone or in combination in the practice of the present invention include, interleukin-1 (IL-1), interleukin-2 (IL-2), interleukin 3 (IL-3), interleukin-4 (IL-4), interleukin-6 (IL-6), interleukin-7 (IL-7), interleukin-9 (IL-9), interleukin 12 (IL-12), interleukin-15 (IL-15), interleukin-21 (IL-21), type I interferons, interferon-α, interferon-β, type II interferons, interferon-γ, stem cell factor (SCF), granulocyte colony stimulating factor (G-CSF) granulocyte macrophage-colony stimulating factor (GM-CSF), interleukin-1α (IL-1α), interleukin-11 (IL-11), MIP-1a, leukemia inhibitory factor (LIF), c-kit ligand, thrombopoietin (TPO) and flt3 ligand. The present invention also includes pharmaceutical compositions in which one or more cytokine, or one or more antibodies capable of binding to a cytokine, or a combination thereof. Cytokines are commercially available from several vendors such as, for example, Genzyme (Framingham, Mass.), Genentech (South San Francisco, Calif.), Amgen (Thousand Oaks, Calif.), or R&D Systems (Minneapolis, Minn.). It is intended, although not always explicitly stated, that molecules having similar biological activity as wild-type or purified cytokines (e.g., recombinantly produced or muteins thereof) are intended to be used within the spirit and scope of the invention.

“Cytokine receptor” refers to receptor molecules that recognize and bind to cytokines. It is intended that the term encompass soluble cytokine receptors as well as cytokine receptors that are cell-bound. In some embodiments, the term refers to interleukin-15 receptor α, which binds to interleukin-15. It is intended that the term also encompass modified cytokine receptor molecules (i.e., “variant cytokine receptors”), including those with substitutions, deletions, and/or additions to the cytokine receptor amino acid and/or nucleic acid sequence. Thus, it is intended that the term encompass wild-type, as well as recombinant, synthetically-produced, and variant cytokine receptors. “Cytokine receptor” refers to all human cytokine receptors within the art that bind one or more cytokine(s), as defined hereinunder, including but not limited to receptors of IL-1, IL-2, IL-3, IL-4, IL-6, IL-7, IL-9, IL-10, IL-12, IL-15, IL-17, IL-21, type I interferons, type II interferons, IFN-α, IFN-β, or IFN-γ.

“Improving immune function in a mammalian subject” refers to the ability of treatment with the compositions of the present invention to successfully reduce or eliminate a disease in a mammalian subject or to prevent its occurrence or recurrence. Diseases include but are not limited to, neoplastic disease, autoimmune disease, or infectious disease or wherein improved immune function provides a therapeutic effect to expand a hematopoietic cell population or improves hematopoietic cell recovery from cell depletion resulting from irradiation or cytotoxic drug treatment, or primary or secondary immunodeficiency in the mammalian subject, or aging. For example, compositions and formulations used to treat or prevent neoplastic disease. include antibodies to cytokines or cytokine antibody complexes or cytokine/cytokine receptor complexes which can serve to interfere with tumor induction; to maintain or improve immune function, for example, increase populations of hematopoietic cells generally or during chemotherapy; and for example to enhance the activity of tumor infiltrating lymphocytes; and/or to reduce chemotherapy induced suppression of NK-cell and lymphokine-activated killer cell cytotoxicity, and lymphocyte mitogenic reactivity in cancer subjects.

“Increasing biological activity” and “biologically active” with regard to cytokine/antibody complex compositions or cytokine/cytokine receptor complex compositions of the present invention refer to the ability of the cytokine or lymphokine molecule to specifically bind to and signal in a hematopoietic cell population, e.g., in a T cell population to expand a subset of the T cell population. “Increasing biological activity” can also refer to cytokine molecules, e.g., type I interferons or type II interferons, which specifically bind to and signal in a non-hematopoietic cell population, e.g., epithelial cells or liver cells. Increasing the biological activity of a cytokine by a cytokine antibody complex or a cytokine/cytokine receptor complex of the present invention includes the ability to expand a T cell population including, but not limited to, expanding CD8⁺T cells and CD4⁺T regulatory cells, expanding CD8⁺T cells, or expanding CD4⁺ T regulatory cells and blocking expansion of CD8⁺ T cells or expanding CD4⁺ and CD8⁺ cells, or expanding naïve T cells (both CD4⁺ T cells and CD8⁺ T cells) or memory T cells, or a combination thereof. Accordingly, the administration of the compounds or agents of the present invention can prevent or delay, to alleviate, or to arrest or inhibit development of the symptoms or conditions associated with neoplastic disease, autoimmune disease, cell depleting radiation or chemotherapy, or infectious disease, in a mammalian subject.

Interferon molecules are grouped in the heterogeneous family of cytokines, originally identified on the basis of their ability to induce cellular resistance to viral infections (Diaz et al., J. Interferon Cytokine Res., 16: 179-180, 1996). “Type I interferon”, for example, interferons α/β, include many members of the interferon α family (interferon α1, α2, ω, and τ) as well as interferon β. “Type II interferon”, for example, interferon γ, is different from type I in its particular mechanisms that regulate its production. Whereas the production of interferons α/β is most efficiently induced in many types of cells upon viral infection, interferon-γ is produced mainly in cells of hematopoietic system, such as T-cells or natural killer cells, upon stimulation by antigens or cytokines, respectively. These two interferon systems are functionally non-redundant in the treatment of disease and in antiviral defense host.

The receptor for IL-15 is comprised of three chains, α, β and γ_{c} the α chain is exclusive for IL-15 whereas the β (CD122) and γ_{c} (CD132) chains are shared with the receptor for IL-2 (Kovanen and Leonard., Immunol Rev 202:67-83, 2004). CD122 is expressed at the highest level on the majority (˜70%) of MP CD8 cells in normal mice, and at low but significant levels on naïve CD8 and MP CD4 cells; virtually no CD122 is expressed on naïve CD4 cells (Zhang et al., Immunity 8(5):591-99, 1998). Reflecting the expression pattern of CD122, IL-15 proved to be essential for the turnover and survival of CD122^{hi} MP CD8 cells. Thus, the generation of IL-15⁻ mice revealed that these mice lacked CD122^{hi} MP CD8 cells Kennedy et al., J Exp Med 191(5): 771-780, 2000). The absence of CD122^{hi} MP CD8 cells appeared to reflect a lack of cell survival, rather than a developmental defect, as CD122^{hi} MP CD8 cells adoptively transferred into IL-15⁻ mice failed to proliferate and disappeared rapidly (Judge et al., J Exp Med 196(7): 935-46, 2002). It should be mentioned that NK cells, which are CD122^{hi}, were also found to be exquisitely dependent on IL-15 for survival. Thus, like CD122^{hi} MP CD8 cells, NK cells are markedly reduced in IL-15⁻ mice (Kennedy, et al. J. Exp. Med. 191: 771-780, 2000). IL-15⁻ mice also show a 50% reduction in numbers of naïve CD8 cells, indicating that IL-15 plays a significant role in sustaining survival of naïve CD8 cells (Kennedy et al., J Exp Med 191(5): 771-780, 2000; Berard et al., J Immunol 170(10): 5018-26, 2003). Unlike CD8 cells, the homeostasis of naïve and MP CD4 cells is not noticeably affected in IL-15⁻ mice (Kennedy et al., J Exp Med 191(5): 771-780, 2000).

The direct role of IL-15 on memory CD8 cells is also indicated by the finding that over-expression of IL-15, as in IL-15 transgenic mice, increases the total numbers of CD122^{hi} MP CD8 cells (Marks-Konczalik et al., Proc Natl Acad Sci USA 97(21):11445-50, 2000; Fehniger et al., J Exp Med 193(2):219-31, 2001). As with other cytokines that signal through γc receptors, IL-15 probably supports survival of memory CD8 cells by upregulating anti-apoptotic molecules such as Bcl-2. The signaling pathways triggered by IL-15 appear to be transmitted via STATS and are negatively modulated by SOCS-1. Thus, increased numbers of MP CD8 cells are present in transgenic mice expressing a constitutively activated form of STATS (Burchill et al., J Immunol 171(11): 5853-64, 2003) and, even more strikingly, in mice where the negative effect of SOCS-1 is abrogated, as in IFNγ⁻ SOCS-1⁻ mice (Ilangumaran et al., J Immunol 171(5): 2435-45). In both cases, naïve CD8 cells seem to display increased sensitivity to IL-15, which causes these cells to undergo spontaneous proliferation and subsequent differentiation into MP cells, this transition being dependent on TCR signaling from contact with self-peptide/MHC ligands Ilangumaran et al., J Immunol 171(5): 2435-45; Davey et al., J Exp Med 202(8):1099-108, 2005).

Although considered a soluble cytokine, IL-15 under in vivo conditions is presented in a cell-associated form bound to the IL-15Rα chain. The essential role of IL-15Rα for presentation of IL-15 was first observed with human cell lines (Dubois et al., Immunity 17(5):537-47, 2002). Subsequent work in mice showed that both IL-15 and IL-15Rα need to be synthesized by the same cell, indicating that IL-15 is pre-associated with the IL-15Rα chain in the cytoplasm prior to expression on the cell surface (Burkett et al., J Exp Med 200(7): 825-34, 2004). This unique mode of presentation explains the paradox that MP CD8 cells transferred to IL-15Rα⁻ mice fail to undergo bystander proliferation in response to Poly I:C (Lodolce et al., J Exp Med 194(8):1187-94, 2001). This model also explains why IL-15Rα⁻ mice lack MP CD8 cells, and confirms the authors' original suggestion that IL-15Rα is required for recognition of IL-15 (Lodolce et al., Immunity 9(5):669-76, 1998). It should be noted that the IL-15Rα chain is expressed on many cell types, including T cells and APC, and is readily upregulated upon activation of these cells, although only non-T cells appear to synthesize IL-15 (Doherty et al., J Immunol 156(2): 735-41, 1996). Although the obligatory role for IL-15Rα expression on APC for presentation of IL-15 is clear, the reason why CD8 cells express IL-15Rα is obscure. Thus, T cell expression of IL-15Rα is largely dispensable for recognition of IL-15 by CD8 cells, and expression of only the β and γ chains of IL-15R on CD8 cells is sufficient for normal responses of memory CD8 cells to IL-15 (Lodolce et al., J Exp Med 194(8):1187-94, 2001; Burkett et al., Proc Natl Acad Sci USA 100(8): 4724-9, 2003). The function of IL-15Rα on CD8 cells remains a mystery, but it could be involved in trans-presentation of soluble IL-15 to other T cells (Dubois et al., Immunity 17(5):537-47, 2002) or possibly for augmenting the activation of APC (Budagian et al., J Biol Chem 279(40):42192-201, 2004).

Under normal conditions, the basal level of IL-15 is probably established by the constitutive production of IL-15 by DC, which synthesize both IL-15 and IL-15Rα (Burkett et al., J Exp Med 200(7): 825-34, 2004). Since production of IL-15 is efficiently induced by IFNs, especially by IFN-I, the question arises whether background production of IFN-I maintain the basal level of IL-15. In support of this idea IFN-I receptor-deficient mice possess less than half the numbers of CD122^{hi} MP CD8 cells found in normal B6 mice and there is even further depletion of CD122^{hi} MP CD8 cells apparent in STAT-1⁻ mice, which are unresponsive to both IFN-I and IFN-γ.

The hematopoietic system is composed of different cell types that perform distinct functions. Many of its diverse function requires coordinated movement of cell surface receptors including ion channels, adhesion surface molecules to coordinate cell-cell interaction, and cytokine receptors. Despite their diverse functional activities, all hematopoietic cells are believed to develop from a multipotent bone marrow hematopoietic stem cell. Such stem cell has been shown to express a surface marker termed CD34. During differentiation, the stem cell gives rise to progenitor cells in each of several specific hematopoietic cell lineages. The progenitor cells then undergo a series of morphological and functional changes to produce mature functionally committed hematopoietic cells.

Among the functions performed by hematopoietic cells, certain cell types are involved exclusively in immunity. For example, lymphocytes, which include T cells, B cells and natural killer (NK) cells, are effectors in immune responses. Monocytes and granulocytes (i.e., neutrophils, basophils and eosinophils) play a role in non-specific forms of defense. Lymphocytes, monocytes and granulocytes are collectively referred to as white blood cells or leukocytes. On the other hand, other hematopoietic cells perform functions that are unrelated to the immune system. For example, erythrocytes are involved in gas transport, and cells of the thrombocytic series are involved in blood clotting.

T cells and B cells recognize antigens and generate an immune response. T cells recognize antigens by heterodimeric surface receptors termed the T cell receptor (TCR). The TCR is associated with a series of polypeptides collectively referred to as CD3 complex. B cells recognize antigens by surface immunoglobulins (Ig), which are also secretory molecules. In addition, a large number of co-stimulatory surface receptors have been identified in T cells and B cells, which augment cellular activation during antigen-induced activation.

In addition to the T cell antigen receptor/CD3 complex (TCR/CD3), other molecules expressed by T cells which mediate an activation signal, include but are not limited to, CD2, CD4, CD5, CD6, CD8, CD18, CD27, CD28, CD43, CD45, CD152 (CTLA-4), CD154, MHC class I, MHC class II, CDw137 (4-1BB), CDw150, and the like (Barclay et al., The Leucocyte Antigen Facts Book, 1997, Second edition, Academic Press; Leucocyte Typing, 1984, Bernard et al. (eds.), Springer-Verlag; Leukocyte Typing II, 1986, Reinherz et al. (eds.), Springer-Verlag; Leukocyte Typing III, 1987, McMichael (ed.), Oxford University Press; Leukocyte Typing IV, 1989, Knapp et al. (eds.), Oxford University Press; CD Antigens, 1996, VI Internet Workshop and Conference on Human Leukocyte Differentiation Antigens. Cell surface antigens that work together with TCR/CD3 are often referred to as co-receptors in the art.

Specific antibodies have been generated against all of the aforementioned T cell surface antigens. Other molecules that bind to the aforementioned T cell surface receptors include antigen-binding antibody derivatives such as variable domains, peptides, superantigens, and their natural ligands such as CD58 (LFA-3) for CD2, HIV gp120 for CD4, CD27L for CD27, CD80 or CD86 for CD28 or CD152, ICAM1, ICAM2 and ICAM3 for CD11a/CD18, 4-1BBL for CDw137.

Activation molecules expressed by B cells, include but are not limited to, surface Ig, CD18, CD19, CD20, CD21, CD22, CD23, CD40, CD45, CD80, CD86 and ICAM1. Similarly, natural ligands of these molecules and antibodies directed to them as well as antibody derivatives can be used to deliver an activation signal to B cells.

“Neoplastic disease”, “cancer”, “malignancy”, “solid tumor” or “hyperproliferative disorder” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” or “solid tumor cell” is understood as a cell having specific structural properties, lacking differentiation and being capable of invasion and metastasis. “Neoplastic disease” or “cancer” refers to all types of cancer or neoplasm or malignant tumors found in mammals, including carcinomas, sarcomas, lymphomas and leukemias. Examples are cancers of the breast, lung, stomach, and oesophagus, brain and nervous system, head and neck, bone, liver, gall bladder, pancreas, colon, genitourinary system, urinary bladder, urinary system, kidney, testes, uterus, ovary, prostate, skin and skin appendices, melanoma, mesothelioma, endocrine system. (see DeVita, et al., (eds.), 2001, Cancer Principles and Practice of Oncology, 6th. Ed., Lippincott Williams & Wilkins, Philadelphia, Pa.; this reference is herein incorporated by reference in its entirety for all purposes).

“Metastatic” refers to tumor cells as defined above which spread to other organs or to distant sites of the same organ.

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

In the context of the cancer, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” refers to tumor cells, e.g., human solid tumor or genitourinary malignancy, that are able to establish secondary tumor lesions in the lungs, liver, bone or brain of immune deficient mice upon injection into the mammary fat pad and/or the circulation of the immune deficient mouse.

### Cancer Treatment

A cytokine antibody complex or a cytokine/cytokine receptor complex is useful in a method of treating disease, for example, neoplastic disease. A “solid tumor” includes, but is not limited to, sarcoma, melanoma, carcinoma, or other solid tumor cancer.

“Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectatic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

### Therapeutic Application of an Antibody Cytokine Complex

As is well understood in the art, biospecific capture reagents include antibodies, binding fragments of antibodies which bind to cytokines or lymphokines (e.g., complete antibody molecules having full length heavy and light chains, or any fragment thereof or affibodies (Affibody, Teknikringen 30, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The hybrid antibodies and hybrid antibody fragments include complete antibody molecules having full length heavy and light chains, or any fragment thereof, e.g., antibody fragments including the Fc region. Chimeric antibodies which have variable regions as described herein and constant regions from various species are also suitable. See, for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like Target objects include any substance which is capable of exhibiting antigenicity and are usually proteins or protein polysaccharides. Examples include receptors, enzymes, hormones, growth factors, peptides, and the like. It should be understood that not only are naturally occurring antibodies suitable for use in accordance with the present disclosure, but engineered antibodies and antibody fragments which are directed to a predetermined object are also suitable.

Antibodies (Abs) that can be subjected to the techniques set forth herein include monoclonal and polyclonal antibodies, and antibody fragments that include the Fc region, such as diabodies, antibody light chains, antibody heavy chains and/or antibody fragments derived from phage or phagemid display technologies. To begin with, an initial antibody is obtained from an originating species. More particularly, the nucleic acid or amino acid sequence of the variable portion of the light chain, heavy chain or both, of an originating species antibody having specificity for a target antigen is needed. The originating species is any species which was used to generate the antibodies or antibody libraries, e.g., rat, mice, rabbit, chicken, monkey, human, and the like. Techniques for generating and cloning monoclonal antibodies are well known to those skilled in the art. After a desired antibody is obtained, the variable regions (V_{H} and V_{L}) are separated into component parts (i.e, frameworks (FRs) and CDRs) using any possible definition of CDRs (e.g., Kabat alone, Chothia alone, Kabat and Chothia combined, and any others known to those skilled in the art). Once that has been obtained, the selection of appropriate target species frameworks is necessary. One embodiment involves alignment of each individual framework region from the originating species antibody sequence with variable amino acid sequences or gene sequences from the target species. Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species can be other than human.

“Treating” refers to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with disease, e.g., neoplastic disease, autoimmune disease, cell depleting radiation or chemotherapy, or infectious disease. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect. “Concomitant administration” of a known cancer therapeutic drug or autoimmune therapeutic drug with a pharmaceutical composition of the present invention means administration of the drug and the antibody or cytokine antibody complex composition or cytokine/cytokine receptor complex composition at such time that both the known drug and the composition of the present invention will have a therapeutic effect. Such concomitant administration can involve concurrent (i.e. at the same time), prior, or subsequent administration of the cancer therapeutic drug or autoimmune therapeutic drug with respect to the administration of a compound of the present invention. A person of ordinary skill in the art, would have no difficulty determining the appropriate timing, sequence and dosages of administration for particular drugs and compositions of the present invention.

“Treating” or “treatment” of disease, e.g., neoplastic disease, autoimmune disease, cell depleting radiation or chemotherapy, or infectious disease, using the methods of the present invention includes preventing the onset of symptoms in a subject that can be at increased risk of disease but does not yet experience or exhibit symptoms of infection, inhibiting the symptoms of infection (slowing or arresting its development), providing relief from the symptoms or side-effects of infection (including palliative treatment), and relieving the symptoms of infection (causing regression).

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding a cytokine, antibody, or cytokine receptor, as described herein, or amino acid sequence of a cytokine, antibody, or cytokine receptor, as described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math, 2: 482, 1981, by the homology alignment algorithm of Needleman and Wunsch, J. Mol. Biol, 48:443, 1970, by the search for similarity method of Pearson and Lipman, Proc. Nat'l. Acad. Sci. USA, 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology. 1995 supplement).

A preferred example of algorithm that is suitable for determining percent sequence identity and sequence similarity are the BLAST and BLAST 2.0 algorithms, which are described in Altschul et al., Nuc. Acids Res, 25:3389-3402, 1977 and Altschul et al., J. Mol. Biol, 215:403-410, 1990, respectively. BLAST and BLAST 2.0 are used, with the parameters described herein, to determine percent sequence identity for the nucleic acids and proteins as embodiments of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA, 89:10915, 1989) alignments (B) of 50, expectation (E) of 10, M=5, N=−4, and a comparison of both strands.

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles as embodiments of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell, 3rd ed., 1994) and Cantor and Schimmel, Biophysical Chemistry Part I: The Conformation of Biological Macromolecules, 1980. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, “Techniques in Biochemistry and Molecular Biology—Hybridization with Nucleic Probes,” Overview of principles of hybridization and the strategy of nucleic acid assays, 1993. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y., 1990.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

Except when noted, “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compositions can be administered. In some embodiments of the present invention, the patient will be suffering from neoplastic disease, autoimmune disease, cell depleting radiation or chemotherapy, infectious disease, or a condition that causes lowered resistance to disease, e.g., HIV. In an exemplary embodiment of the present invention, to identify subject patients for treatment with a pharmaceutical composition comprising one or more cytokine antibody complexes according to the methods, accepted screening methods are employed to determine the status of an existing disease or condition in a subject or risk factors associated with a targeted or suspected disease or condition. These screening methods include, for example, examinations to determine whether a subject is suffering from an disease. These and other routine methods allow the clinician to select subjects in need of therapy.

After selecting suitable frame work region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

Assembly of a physical antibody or antibody fragment library is preferably accomplished using synthetic oligonucleotides. In one example, oligonucleotides are designed to have overlapping regions so that they could anneal and be filled in by a polymerase, such as with polymerase chain reaction (PCR). Multiple steps of overlap extension are performed in order to generate the V_{L} and V_{H} gene inserts. Those fragments are designed with regions of overlap with human constant domains so that they could be fused by overlap extension to produce full length light chains and Fd heavy chain fragments. The light and heavy Fd chain regions can be linked together by overlap extension to create a single Fab library insert to be cloned into a display vector. Alternative methods for the assembly of the humanized library genes can also be used. For example, the library can be assembled from overlapping oligonucleotides using a Ligase Chain Reaction (LCR) approach. Chalmers et al., Biotechniques, 30-2: 249-252, 2001.

Various forms of antibody fragments can be generated and cloned into an appropriate vector to create a hybrid antibody library or hybrid antibody fragment library. For example variable genes can be cloned into a vector that contains, in-frame, the remaining portion of the necessary constant domain. Examples of additional fragments that can be cloned include whole light chains, the Fc portion of heavy chains, or fragments that contain both light chain and heavy chain Fc coding sequence.

Any selection display system can be used in conjunction with a library according to the present disclosure. Selection protocols for isolating desired members of large libraries are known in the art, as typified by phage display techniques. Such systems, in which diverse peptide sequences are displayed on the surface of filamentous bacteriophage have proven useful for creating libraries of antibody fragments (and the nucleotide sequences that encode them) for the in vitro selection and amplification of specific antibody fragments that bind a target antigen. Scott et al., Science, 249: 386, 1990. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage or T7 capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward. Methods for the construction of bacteriophage antibody display libraries and lambda phage expression libraries are well known in the art. McCafferty et al., Nature, 348: 552, 1990; Kang et al., Proc. Natl. Acad. Sci. U.S.A., 88: 4363, 1991.

The present invention further relates to antibodies and T-cell antigen receptors (TCR) which specifically bind the cytokines or lymphokines of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention and include, but are not limited to, single-chain antibodies, disulfide-linked Fvs (sdFv), fragments comprising the Fc domain, and fragments comprising either a V_{L} or V_{H} domain. The antibodies can be from any animal origin including birds and mammals. Preferably, the antibodies are human, murine, rabbit, goat, guinea pig, camel, horse, or chicken.

Antigen-binding antibody fragments, including single-chain antibodies, can comprise the variable region(s) alone or in combination with the entire or partial of the following: hinge region, CH₁, CH₂, and CH₃ domains. Also included in the invention are any combinations of variable region(s) and hinge region, CH₁, CH₂, and CH₃ domains. The present invention further includes monoclonal, polyclonal, chimeric, humanized, and human monoclonal and human polyclonal antibodies which specifically bind the polypeptides of the present invention. The present invention further includes antibodies which are anti-idiotypic to the antibodies of the present invention.

The antibodies of the present invention can be monospecific, bispecific, trispecific or of greater multispecificity. Multispecific antibodies can be specific for different epitopes of a polypeptide of the present invention or can be specific for both a polypeptide of the present invention as well as for heterologous compositions, such as a heterologous polypeptide or solid support material. See, e.g., WO 93/17715; WO 92/08802; WO 91/00360; WO 92/05793; Tutt et al., J. Immunol. 147: 60-69, 1991; U.S. Pat. Nos. 5,573,920; 4,474,893; 5,601,819; 4,714,681; 4,925,648, each incorporated herein by reference in their entirety and for all purposes; Kostelny et al., J. Immunol. 148: 1547-1553, 1992.

Antibodies of the present invention can be described or specified in terms of the epitope(s) or portion(s) of a polypeptide of the present invention which are recognized or specifically bound by the antibody. The epitope(s) or polypeptide portion(s) can be specified as described herein, e.g., by N-terminal and C-terminal positions, by size in contiguous amino acid residues. Antibodies which specifically bind any epitope or polypeptide of the present invention can also be excluded. Therefore, the present invention includes antibodies that specifically bind polypeptides of the present invention, and allows for the exclusion of the same.

Antibodies of the present invention can also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention can also be described or specified in terms of their binding affinity. Preferred binding affinities include those with a dissociation constant or K_{d} less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies to cytokines that form a cytokine antibody complex have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow and Lane, supra, incorporated herein by reference in its entirety and for all purposes.

The antibodies of the present invention can be used either alone or in combination with other compositions. The antibodies can further be recombinantly fused to a heterologous polypeptide at the N- or C-terminus or chemically conjugated (including covalently and non-covalently conjugations) to polypeptides or other compositions. For example, antibodies can be recombinantly fused or conjugated to cytokine or lymphokine molecules. For example, antibodies of the present invention can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387, each incorporated herein by reference in their entirety and for all purposes.

The antibodies to cytokines or lymphokines of the present invention can be prepared by any suitable method known in the art. For example, cytokines or lymphokines of the present invention or an antigenic fragment thereof can be administered to an animal in order to induce the production of sera containing polyclonal antibodies. The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art and taught in Harlow and Lane, supra; Hammerling et al., Monoclonal Antibodies and T-Cell Hybridomas, 563-681, 1981, said references incorporated by reference in their entireties. Fab and F(ab′)₂ fragments can be produced by proteolytic cleavage, using enzymes such as papain (to produce Fab fragments) or pepsin (to produce F(ab′)₂ fragments).

Alternatively, antibodies to cytokines or lymphokines can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727 and 5,733,743, each incorporated herein by reference in their entirety and for all purposes.

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce antibody fragments including the Fc region of the antibody can be employed using methods known in the art. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Examples of techniques which can be used to produce single-chain Fvs and antibodies include those described in U.S. Pat. Nos. 4,946,778 and 5,258,498, each incorporated herein by reference in their entirety and for all purposes; Huston et al., Methods in Enzymology, 203: 46-88, 1991; Shu, L. et al., PNAS 90: 7995-7999, 1993; and Skerra et al., Science 240: 1038-1040, 1988. For some uses, including in vivo use of antibodies in humans and in vitro detection assays, it may be preferable to use chimeric, humanized, or human antibodies. Methods for producing chimeric antibodies are known in the art. See e.g., Morrison, Science 229: 1202, 1985; Oi et al., BioTechniques 4: 214, 1986; Gillies et al., J. Immunol. Methods, 125: 191-202, 1989; and U.S. Pat. No. 5,807,715. Antibodies can be humanized using a variety of techniques including CDR-grafting (EP 0 239 400; WO 91/09967; and U.S. Pat. Nos. 5,530,101 and 5,585,089), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). Human antibodies can be made by a variety of methods known in the art including phage display methods described above. See also, U.S. Pat. Nos. 4,444,887; 4,716,111; 5,545,806; and 5,814,318; and WO 98/46645; WO 98/50433; WO 98/24893; WO 98/16654; WO 96/34096; WO 96/33735; and WO 91/10741, each incorporated herein by reference in their entirety and for all purposes.

Further included in the present invention are antibodies recombinantly fused or chemically conjugated (including both covalently and non-covalently conjugations) to a cytokine or lymphokine of the present invention. The antibodies can be specific for antigens other than cytokines or lymphokines of the present invention. For example, antibodies can be used to target the cytokines or lymphokines of the present invention to particular cell types, either in vitro or in vivo, by fusing or conjugating the polypeptides of the present invention to antibodies specific for particular cell surface receptors. Antibodies fused or conjugated to the polypeptides of the present invention can also be used in in vitro immunoassays and purification methods using methods known in the art. See e.g., Harbor et al., supra, and WO 93/21232; EP 0 439 095; Naramura et al., Immunol. Lett. 39: 91-99, 1994; U.S. Pat. No. 5,474,981, incorporated herein by reference in its entirety and for all purposes; Gillies et al., PNAS 89: 1428-1432, 1992; Fell et al., J. Immunol. 146: 2446-2452, 1991.

The present invention further includes compositions comprising the cytokines or lymphokines of the present invention fused or conjugated to antibody domains of an antibody Fc region, or portion thereof. The antibody portion fused to a polypeptide of the present invention can comprise the hinge region, CH₁ domain, CH₂ domain, and CH₃ domain or any combination of whole domains or portions thereof. The cytokines or lymphokines of the present invention can be fused or conjugated to the above antibody portions to increase the in vivo half life of the polypeptides or for use in immunoassays using methods known in the art. The polypeptides can also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the cytokine antibody complex of the present invention to antibody portions are known in the art. See, e.g., U.S. Pat. Nos. 5,336,603; 5,622,929; 5,359,046; 5,349,053; 5,447,851; 5,112,946; EP 0 307 434, EP 0 367 166; WO 96/04388; and WO 91/06570, each incorporated herein by reference in their entirety and for all purposes; Ashkenazi et al., PNAS, 88: 10535-10539, 1991; Zheng et al., J. Immunol., 154: 5590-5600, 1995; and Vil et al., PNAS, 89: 11337-11341, 1992.

The invention further relates to antibodies which act as agonists of the cytokines or lymphokines of the present invention. For example, the present invention includes antibodies which activate the receptor for cytokines or lymphokines. These antibodies can act as agonists for either all or less than all of the biological activities affected by ligand-mediated receptor activation. The antibodies can be specified as agonists for biological activities comprising specific activities disclosed herein. The above antibody agonists can be made using methods known in the art. See e.g., WO 96/40281; U.S. Pat. No. 5,811,097, each incorporated herein by reference in their entirety and for all purposes; Deng et al., Blood 92: 1981-1988, 1998; Chen, et al., Cancer Res., 58: 3668-3678, 1998; Harrop et al., J. Immunol. 161: 1786-1794, 1998; Zhu et al., Cancer Res., 58: 3209-3214, 1998; Yoon, et al., J. Immunol., 160: 3170-3179, 1998; Prat et al., J. Cell. Sci., 111: 237-247, 1998; Pitard et al., J. Immunol. Methods, 205: 177-190, 1997; Liautard et al., Cytokine, 9: 233-241, 1997; Carlson et al., J. Biol. Chem., 272: 11295-11301, 1997; Taryman et al., Neuron, 14: 755-762, 1995; Muller et al., Structure, 6: 1153-1167, 1998; Bartunek et al., Cytokine, 8: 14-20, 1996. As discussed above, antibodies to cytokines or lymphokines on metastatic cells can, in turn, be utilized to generate anti-idiotype antibodies that “mimic” polypeptides as embodiments of the invention using techniques well known to those skilled in the art. (See, e.g., Greenspan et al., FASEB J. 7: 437-444, 1989 and Nissinoff, J. Immunol. 147: 2429-2438, 1991). For example, antibodies which bind to and competitively inhibit polypeptide multimerization and/or binding of a polypeptide as embodiments of the invention to ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide as embodiments of the invention and/or to bind its ligands/receptors, and thereby block its biological activity.

“Inhibitors,” “activators,” and “modulators” of cytokine receptor on metastatic cells or infected cells, or Fc receptor on macrophage cells are used to refer to inhibitory, activating, or modulating molecules, respectively, identified using in vitro and in vivo assays for receptor binding or signaling, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate the activity of cytokines or lymphokines on cell receptors, e.g., antagonists. Activators are agents that, e.g., bind to, stimulate, increase, open, activate, facilitate, enhance activation, sensitize or up regulate the activity of cytokines or lymphokines on cell receptors, e.g., agonists. Modulators include agents that, e.g., alter the interaction of cytokine or lymphokine receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring cytokines or lymphokines, e.g., with altered activity, as well as naturally occurring and synthetic ligands, antagonists, agonists, small chemical molecules and the like. Such assays for inhibitors and activators include, e.g., applying putative modulator compounds to a cell expressing a cytokine or lymphokine receptor and then determining the functional effects on cytokine or lymphokine signaling, as described herein. Samples or assays comprising a receptor that are treated with a potential activator, inhibitor, or modulator are compared to control samples without the inhibitor, activator, or modulator to examine the extent of activation or inhibition. Control samples (untreated with inhibitors) can be assigned a relative receptor activity value of 100%. Inhibition of receptor is achieved when the receptor activity value relative to the control is about 80%, optionally 50% or 25-0%. Activation of receptor is achieved when the receptor activity value relative to the control is 110%, optionally 150%, optionally 200-500%, or 1000-3000% higher.

The ability of a molecule to bind to cytokine or lymphokine receptor can be determined, for example, by the ability of the putative ligand to bind to activated receptor immunoadhesin coated on an assay plate. Specificity of binding can be determined by comparing binding to non-activated receptor.

In one embodiment, antibody binding to cytokine or lymphokine receptor can be assayed by either immobilizing the ligand or the receptor. For example, the assay can include immobilizing cytokine or lymphokine receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

### Expression of Recombinant Antibodies and Receptors

Chimeric, humanized and human antibodies and receptors to cytokines or lymphokines, e.g., cytokine antibody complexes and cytokine receptor complexes, are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. See U.S. Application No. 20020199213 incorporated herein by reference in its entirety and for all purposes.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins and cytokine receptors or fragments thereof. See Winnacker, From Genes To Clones, VCH Publishers, NY, 1987. A number of suitable host cell lines capable of secreting intact heterologous proteins have been developed in the art, and include Chinese hamster ovary (CHO) cell lines, various COS cell lines, HeLa cells, L cells and myeloma cell lines. Preferably, the cells are nonhuman. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Preferred expression control sequences are promoters derived from endogenous genes, cytomegalovirus, SV40, adenovirus, bovine papillomavirus, and the like. Co, et al., J Immunol. 148: 1149, 1992.

Alternatively, antibody and cytokine receptor coding sequences can be incorporated in transgenes for introduction into the genome of a transgenic animal and subsequent expression in the milk of the transgenic animal. See, e.g., U.S. Pat. Nos. 5,741,957; 5,304,489; and 5,849,992, each incorporated herein by reference in their entirety and for all purposes. Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., Molecular Cloning). For production of transgenic animals, transgenes can be microinjected into fertilized oocytes, or can be incorporated into the genome of embryonic stem cells, and the nuclei of such cells transferred into enucleated oocytes.

Once expressed, collections of antibodies and receptors are purified from culture media and host cells. Antibodies and receptors can be purified according to standard procedures of the art, including HPLC purification, column chromatography, gel electrophoresis and the like. Usually, antibody chains are expressed with signal sequences and are thus released to the culture media. However, if antibody chains are not naturally secreted by host cells, the antibody chains can be released by treatment with mild detergent. Antibody chains can then be purified by conventional methods including ammonium sulfate precipitation, affinity chromatography to immobilized target, column chromatography, gel electrophoresis and the like (see generally Scopes, Protein Purification, Springer-Verlag, N.Y., 1982).

The above methods result in libraries of nucleic acid sequences encoding antibody chains having specific affinity for a chosen target. The libraries of nucleic acids typically have at least 5, 10, 20, 50, 100, 1000, 10⁴, 10⁵, 10⁶, 10⁷, 10⁸, or 10⁹ different members. Usually, no single member constitutes more than 25 or 50% of the total sequences in the library. Typically, at least 25, 50%, 75, 90, 95, 99 or 99.9% of library members encode antibody chains with specific affinity for the target molecules. In the case of double chain antibody libraries, a pair of nucleic acid segments encoding heavy and light chains respectively is considered a library member. The nucleic acid libraries can exist in free form, as components of any vector or transfected as a component of a vector into host cells.

The nucleic acid libraries can be expressed to generate polyclonal libraries of antibodies having specific affinity for a target. The composition of such libraries is determined from the composition of the nucleotide libraries. Thus, such libraries typically have at least 5, 10, 20, 50, 100, 1000, 10⁴, 10⁵, 10⁶, 10⁷, 10⁸, or 10⁹ members with different amino acid composition. Usually, no single member constitutes more than 25 or 50% of the total polypeptides in the library. The percentage of antibody chains in an antibody chain library having specific affinity for a target is typically lower than the percentage of corresponding nucleic acids encoding the antibody chains. The difference is due to the fact that not all polypeptides fold into a structure appropriate for binding despite having the appropriate primary amino acid sequence to support appropriate folding. In some libraries, at least 25, 50, 75, 90, 95, 99 or 99.9% of antibody chains have specific affinity for the target molecules. Again, in libraries of multi-chain antibodies, each antibody (such as a Fab or intact antibody) is considered a library member. The different antibody chains differ from each other in terms of fine binding specificity and affinity for the target. Some such libraries comprise members binding to different epitopes on the same antigen. Some such libraries comprises at least two members that bind to the same antigen without competing with each other.

Polyclonal libraries of human antibodies resulting from the above methods are distinguished from natural populations of human antibodies both by the high percentages of high affinity binders in the present libraries, and in that the present libraries typically do not show the same diversity of antibodies present in natural populations. The reduced diversity in the present libraries is due to the nonhuman transgenic animals that provide the source materials not including all human immunoglobulin genes. For example, some polyclonal antibody libraries are free of antibodies having lambda light chains. Some polyclonal antibody libraries as embodiments of the invention have antibody heavy chains encoded by fewer than 10, 20, 30 or 40 V_{H} genes. Some polyclonal antibody libraries as embodiments of the invention have antibody light chains encoded by fewer than 10, 20, 30 or 40 V_{L} genes.

### Modified Antibodies and Receptors

Also included in the invention are modified antibodies to cytokines or lymphokines and receptors to cytokines or lymphokines, for increasing T cell populations and for treatment of disease.

“Modified antibody” refers to antibodies and antibody fragments optimized chemically or by molecular engineering into different formats, including but not limited to diabodies, triabodies or bispecific antibodies, pegylated derivatives, variants derived from molecular evolution to enhance affinity, stability, or valency. Modified antibodies also include formats such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody.

The cytokine/antibody complex or cytokine/cytokine receptor complex can be used to modify a given biological response or create a biological response (e.g., to recruit effector cells). The drug moiety is not to be construed as limited to classical chemical therapeutic agents. For example, the drug moiety can be a protein or polypeptide possessing a desired biological activity. Such proteins can include, for example, an enzymatically active toxin, or active fragment thereof, such as abrin, ricin A, pseudomonas exotoxin, or diphtheria toxin; a protein such as tumor necrosis factor or interferon-alpha; or, biological response modifiers such as, for example, lymphokines, interleukin-1 (“IL-1”), interleukin-2 (“IL-2”), interleukin-4 (“IL-4”), interleukin-6 (“IL-6”), interleukin-7 (“IL-7”), interleukin-15 (“IL-15”), granulocyte macrophage colony stimulating factor (“GM-CSF”), granulocyte colony stimulating factor (“G-CSF”), or other growth factors. Other derivatives can include antibody fusion proteins with apoptosis inducing moieties such as TRAIL, tumor necrosis factor-related apoptosis-inducing ligand, and reporter molecules such as luciferase or fluorescent probes and nano-particles for non-invasive imaging or targeted delivery of pay-load molecules to sites with tumor burden and micro- and macro-metastases.

In certain embodiments of the invention, the cytokine/antibody complex or cytokine/cytokine receptor complex, for example, can be coupled or conjugated to one or more therapeutic or cytotoxic moieties. As used herein, “cytotoxic moiety” simply means a moiety that inhibits cell growth or promotes cell death when proximate to or absorbed by a cell. Suitable cytotoxic moieties in this regard include radioactive agents or isotopes (radionuclides), chemotoxic agents such as differentiation inducers, inhibitors and small chemotoxic drugs, toxin proteins and derivatives thereof, as well as nucleotide sequences (or their antisense sequence). Therefore, the cytotoxic moiety can be, by way of non-limiting example, a chemotherapeutic agent, a photoactivated toxin or a radioactive agent.

In general, therapeutic agents can be conjugated to the cytokine/antibody complex or cytokine/cytokine receptor complex compositions, for example a cytokine antibody complex alone or in combination with another therapeutic agent, by any suitable technique, with appropriate consideration of the need for pharmokinetic stability and reduced overall toxicity to the patient. A, alone or in combination with another therapeutic agent, can be coupled to a suitable antibody moiety either directly or indirectly (e.g. via a linker group). A direct reaction between a cytokine or lymphokine and an antibody is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity can also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups can be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues (see, e.g., U.S. Pat. No. 4,671,958).

As an alternative coupling method, cytotoxic agents can be coupled to the antibodies and to the cytokine antibody complex compositions as embodiments of the invention, for example, through an oxidized carbohydrate group at a glycosylation site, as described in U.S. Pat. Nos. 5,057,313 and 5,156,840. Yet another alternative method of coupling the antibody and antibody compositions to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytokine, lymphokine, or cytotoxic moiety is more potent when free from the antibody portion of the immunoconjugates of the present invention, it can be desirable to use a linker group which is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It can be desirable to couple more than one therapeutic, cytokine, lymphokine, or cytotoxic and/or imaging moiety to a cytokine/antibody complex or cytokine/cytokine receptor complex compositions. By poly-derivatizing the antibodies, several therapeutic and/or cytotoxic strategies can be simultaneously implemented, an antibody can be made useful as a contrasting agent for several visualization techniques, or a therapeutic antibody can be labeled for tracking by a visualization technique. In one embodiment, multiple molecules of a cytotoxic moiety are coupled to one antibody molecule. In another embodiment, more than one type of moiety can be coupled to one antibody. For instance, a therapeutic moiety, such as a cytokine or a lymphokine, can be conjugated to an antibody in conjunction with a chemotoxic or radiotoxic moiety, to increase the effectiveness of the chemo- or radiotoxic therapy, as well as lowering the required dosage necessary to obtain the desired therapeutic effect. Regardless of the particular embodiment, immunoconjugates with more than one moiety can be prepared in a variety of ways. For example, more than one moiety can be coupled directly to an antibody molecule, or linkers that provide multiple sites for attachment (e.g., dendrimers) can be used. Alternatively, a carrier with the capacity to hold more than one cytotoxic moiety can be used.

As explained above, a carrier can bear the agents in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier can also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful in chemotoxic therapeutic embodiments, as they can allow the therapeutic compositions to gradually release a chemotoxic moiety over time while concentrating it in the vicinity of the target cells.

Preferred radionuclides for use as cytotoxic moieties are radionulcides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other .beta.-radiation emitting nuclides, which have an effective range of several millimeters. ¹²³I, ¹²⁵I, ¹³¹I, or ²¹¹At can be conjugated to antibody moieties for use in the compositions and methods utilizing any of several known conjugation reagents, including lodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope can be utilized in the recited iodo-reagents. Other radionuclides can be conjugated to the cytokine/antibody complex or cytokine/cytokine receptor complex compositions by suitable chelation agents known to those of skill in the nuclear medicine arts.

Preferred chemotoxic agents include small-molecule drugs such as methotrexate, and pyrimidine and purine analogs. Preferred chemotoxin differentiation inducers include phorbol esters and butyric acid. Chemotoxic moieties can be directly conjugated to the cytokine/antibody complex or cytokine/cytokine receptor complex compositions via a chemical linker, or can encapsulated in a carrier, which is in turn coupled to the cytokine/antibody complex or cytokine/cytokine receptor complex composition.

Preferred toxin proteins for use as cytotoxic moieties include ricin, abrin, diphtheria toxin, cholera toxin, gelonin, Pseudomonas exotoxin, Shigella toxin, pokeweed antiviral protein, and other toxin proteins known in the medicinal biochemistry arts. As these toxin agents can elicit undesirable immune responses in the patient, especially if injected intravascularly, it is preferred that they be encapsulated in a carrier for coupling to the cytokine/antibody complex or cytokine/cytokine receptor complex compositions.

The cytotoxic moiety of the immunotoxin can be a cytotoxic drug or an enzymatically active toxin of bacterial or plant origin, or an enzymatically active fragment (“A chain”) of such a toxin. Enzymatically active toxins and fragments thereof used are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the antibodies are conjugated to small molecule anticancer drugs. Conjugates of the monoclonal antibody and such cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as his (p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin can be joined to the Fab fragment of antibodies.

Advantageously, the cytokine/antibody complex or cytokine/cytokine receptor complex compositions specifically binding the external domain of the cytokine or antibody can be conjugated to ricin A chain. Most advantageously the ricin A chain is deglycosylated and produced through recombinant means. An advantageous method of making the ricin immunotoxin is described in Vitetta et al., Science 238, 1098, 1987, which is incorporated by reference in its entirety.

The term “contacted” when applied to a cell is used herein to describe the process by which an antibody, antibody composition, cytotoxic agent or moiety, gene, protein and/or antisense sequence, is delivered to a target cell or is placed in direct proximity with the target cell. This delivery can be in vitro or in vivo and can involve the use of a recombinant vector system.

In another aspect, the present invention features an cytokine/antibody complex or cytokine/cytokine receptor complex, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates which include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, duocarmycin, saporin, dihydroxy anthracin didne, mitoxantrone, mithramycin, actinomycin D, 1-dehydrotestosterone, glucocorticoids, procaine, tetracaine, lidocaine, propranolol, and puromycin and analogs or homologs thereof.

Suitable therapeutic agents for forming immunoconjugates include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a further embodiment, the therapeutic agent is doxorubicin (adriamycin), cisplatin bleomycin sulfate, carmustine, chlorambucil, cyclophosphamide hydroxyurea or ricin A.

cytokine/antibody complex or cytokine/cytokine receptor complex compositions also can be conjugated to a radiotoxin, e.g., radioactive iodine, to generate cytotoxic radiopharmaceuticals for treating, for example, a cancer. The cytokine/antibody complex or cytokine/cytokine receptor complex can be used to modify a given biological response, and the drug moiety is not to be construed as limited to classical chemical therapeutic agents. For example, the drug moiety can be a protein or polypeptide possessing a desired biological activity. Such proteins can include, for example, an enzymatically active toxin, or active fragment thereof, such as abrin, ricin A, pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-4 (“IL-4”), interleukin-6 (“IL-6”), interleukin-7 (“IL-7”), interleukin-15 (“IL-15”), granulocyte macrophage colony stimulating factor (“GM-CSF”), granulocyte colony stimulating factor (“G-CSF”), or other growth factors.

Techniques for conjugating such therapeutic moiety to antibodies are well known. See, e.g., Amon et al., “Monoclonal Antibodies For Immunotargeting Of Drugs In Cancer Therapy”, in Reisfeld et al., eds., Monoclonal Antibodies And Cancer Therapy, Alan R. Liss, Inc., pp. 243-56, 1985); Hellstrom et al., “Antibodies For Drug Delivery”, in Controlled Drug Delivery 2nd Ed., Marcel Dekker, Inc., Robinson et al., eds., pp. 623-53, 1987; Thorpe, “Antibody Carriers Of Cytotoxic Agents In Cancer Therapy: A Review”, in Monoclonal Antibodies '84: Biological And Clinical Applications, Pinchera et al., eds., pp. 475-506, 1985; “Analysis, Results, And Future Prospective Of The Therapeutic Use Of Radiolabeled Antibody In Cancer Therapy”, in Monoclonal Antibodies For Cancer Detection And Therapy, Baldwin et al., eds., Academic Press, pp. 303-16 1985, and Thorpe et al., “The Preparation And Cytotoxic Properties Of Antibody-Toxin Conjugates”, Immunol. Rev., 62: 119-58, 1982.

### Uses of Cytokine/Antibody Complex or Cytokine/Cytokine Receptor Complex Compositions

Each of the cytokine/antibody complex or cytokine/cytokine receptor complex compositions, e.g., cytokine antibody complexes that stimulate expansion of T cell populations, identified herein can be used in numerous ways. The following description should be considered exemplary and utilizes known techniques.

A cytokine/antibody complex or cytokine/cytokine receptor complex composition can be used to assay protein levels in a biological sample using antibody-based techniques. For example, protein expression in tissues can be studied with classical immunohistological methods. Jalkanen et al., J. Cell. Biol. 101: 976-985, 1985; Jalkanen et al., J. Cell. Biol. 105: 3087-3096, 1987. Other antibody-based methods useful for detecting protein gene expression include immunoassays, such as the enzyme linked immunosorbent assay (ELISA) and the radioimmunoassay (MA). Suitable antibody assay labels are known in the art and include enzyme labels, such as, glucose oxidase, and radioisotopes or other radioactive agent, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying secreted protein levels in a biological sample, proteins or antibody compositions can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the antibody by labeling of nutrients for the relevant scFv clone.

A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein. In vivo tumor imaging is described in Burchiel et al., Tumor Imaging: The Radiochemical Detection of Cancer 13, 1982.

Moreover, cytokine/antibody complex or cytokine/cytokine receptor complex compositions can be used to treat disease. For example, patients can be administered a cytokine antibody complex compositions of the present invention in an effort to enhance the expansion of a T cell population, to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, cytokine/antibody complex or cytokine/cytokine receptor complex compositions can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane receptor.

### Treatment Regimes

The invention provides pharmaceutical compositions comprising cytokine/antibody complex or cytokine/cytokine receptor complex compositions for the treatment of disease, e.g., neoplastic disease, autoimmune disease, cell depleting radiation or chemotherapy, or infectious disease, formulated together with a pharmaceutically acceptable carrier. Some compositions include a combination of multiple (e.g., two or more) cytokine/antibody complex or cytokine/cytokine receptor complex compositions.

In prophylactic applications, pharmaceutical compositions or medicaments are administered to a patient susceptible to, or otherwise at risk of a disease or condition (e.g., neoplastic disease, autoimmune disease, cell depleting radiation or chemotherapy, or infectious disease) in an amount sufficient to eliminate or reduce the risk of recurrence of the a disease or condition, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient anti-proliferative response has been achieved. Typically, the anti-proliferative response is monitored and repeated dosages are given if the anti-proliferative response starts to wane.

### Effective Dosages

Effective doses of the cytokine/antibody complex or cytokine/cytokine receptor complex compositions, e.g., antibodies to cytokine or lymphokine, for the treatment of diseases, e.g., neoplastic disease, autoimmune disease, cell depleting radiation or chemotherapy, or infectious disease, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

### Routes of Administration

Cytokine/antibody complex or cytokine/cytokine receptor complex compositions for inducing an immune response, e.g., cytokine/antibody complex or cytokine/cytokine receptor complex for the treatment of disease, e.g., neoplastic disease, autoimmune disease, cell depleting radiation or chemotherapy, or infectious disease, can be administered by parenteral, topical, intravenous, oral, subcutaneous, intraarterial, intracranial, intraperitoneal, intranasal or intramuscular means for prophylactic as inhalants for antibody preparations targeting brain lesions, and/or therapeutic treatment. The most typical route of administration of an cytokine antibody complex composition is subcutaneous although other routes can be equally effective. The next most common route is intramuscular injection. This type of injection is most typically performed in the arm or leg muscles. In some methods, agents are injected directly into a particular tissue where deposits have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

Cytokine/antibody complex or cytokine/cytokine receptor complex compositions can optionally be administered in combination with other agents that are at least partly effective in treating various diseases including various immune-related diseases. In the case of tumor metastasis to the brain, agents can also be administered in conjunction with other agents that increase passage of the agents across the blood-brain barrier (BBB).

### Formulation

Cytokine/antibody complex or cytokine/cytokine receptor complex compositions for inducing an immune response, for the treatment of diseases, e.g., neoplastic disease, autoimmune disease, cell depleting radiation or chemotherapy, or infectious disease, are often administered as pharmaceutical compositions comprising an active therapeutic agent, i.e., and a variety of other pharmaceutically acceptable components. (See Remington's Pharmaceutical Science, 15^{th} ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions that are embodiments of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of the cytokine/antibody complex or cytokine/cytokine receptor complex compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1.

### Kits

Also within the scope of the invention are kits comprising the cytokine/antibody complex or cytokine/cytokine receptor complex compositions (e.g., monoclonal antibodies, human sequence antibodies, human antibodies, multispecific and bispecific molecules) and instructions for use. The kit can further contain a least one additional reagent, or one or more additional human antibodies (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

### EXEMPLARY EMBODIMENTS

### Example 1

### T Cell Proliferation in Mice Following Injection of Recombinant Mouse IL-2

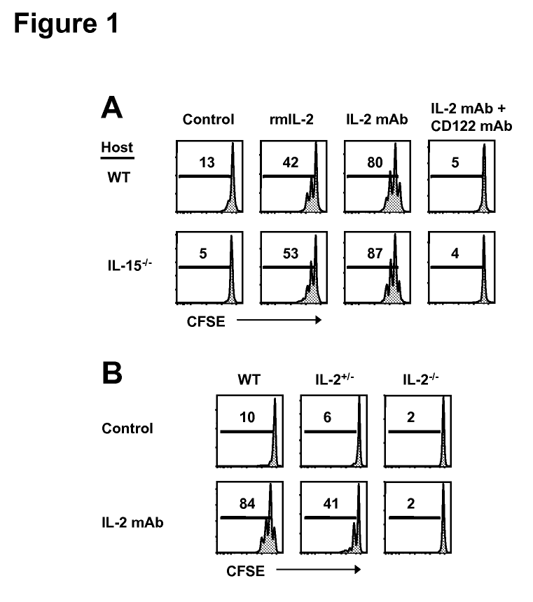

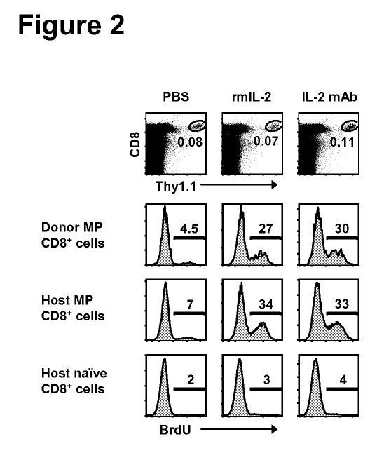

Previous studies have shown that the turnover of MP CD8⁺ cells in vivo can be increased by injecting either IL-2 or IL-2 mAb (FIG. 1). For IL-2, proliferation of CD8⁺ cells in vivo, measured by dilution of the dye CFSE (FIG. 1A, B) or incorporation of bromodeoxyuridine (BrdU) (FIG. 2), was prominent after injection of recombinant mouse IL-2 (rmIL-2) and was largely restricted to MP CD8⁺ cells, both for host and adoptively-transferred purified CD8⁺ cells. In contrast, stimulation of naïve T cells, as defined by low expression of CD122 and CD44, was minimal (FIG. 2). Confirming previous findings, even greater proliferation occurred following injection of IL-2 mAb, specifically by the anti-mouse IL-2 mAb S4B6 (FIGS. 1 and 2). Ku et al., Science 288: 675, 2000; Kamimura et al., J Immunol 173: 6041, 2004. This effect was also seen in IL-15^{-/-} hosts and was blocked by CD122 mAb (FIG. 1A), confirming that the effector cytokine for proliferation is not IL-15 but nevertheless stimulates via CD122. Ku et al., Science 288: 675, 2000; Kamimura et al., J Immunol 173: 6041, 2004.

FIG. 1 shows stimulation of MP CD8⁺ cells in vivo by IL-2 or IL-2 mAb. CFSE-labeled purified Thy1.1 MP (CD44^{hi} CD122^{hi}) CD8⁺T cells were transferred intravenously (iv) to (A) wild-type (WT) or IL-15^{-/-} mice, which then received daily intraperitoneal (ip) injections of PBS, rmIL-2, S4B6 IL-2 mAb, or IL-2 mAb plus CD122 mAb, or to (B) WT, IL-2^{+/-} or IL-2^{-/-} mice, followed by daily injections of S4B6 IL-2 mAb or control mAb. Donor cells were analyzed on day 7 by flow cytometry. Numbers represent percentages of divided (CFSE^{lo}) donor Thy1.1⁺CD8⁺ cells. All data in this and the following figures are representative of at least 2 separate experiments.

FIG. 2 shows proliferation of MP CD8⁺ cells in vivo in response to IL-2 or IL-2 mAb. (A) Purified Thy1.1-marked MP CD44^{hi} CD122^{hi} CD8⁺T cells were transferred intravenously (iv) to normal B6 (Thy1.2) mice. Subsequently, host mice received daily intraperitoneal (ip) injections of PBS, 1.5 μg rmIL-2, or 0.5 mg S4B6 IL-2 mAb for 1 week. To measure T cell turnover, BrdU was given in the drinking water for the last 3 days. Lymph node (LN) and spleen cells were isolated after 7 days and analyzed by flow cytometry. Numbers represent percentages of donor Thy1.1⁺ CD8⁺ cells (top row) or BrdU⁺ cells (lower rows).

### Example 2

### T Cell Proliferation in Response to Cytokine Antibody Complex is Abolished in IL-2^{-/-} Mice or Reduced in IL-2^{+/-} Mice

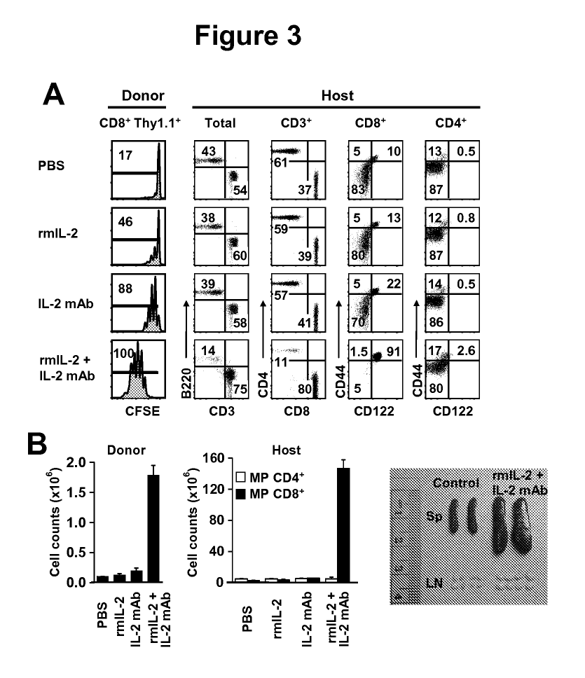

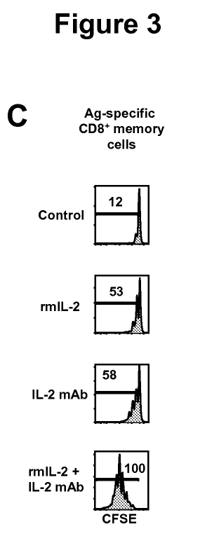

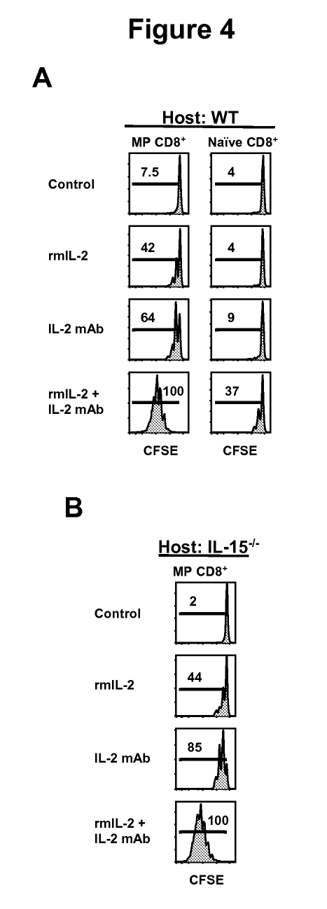

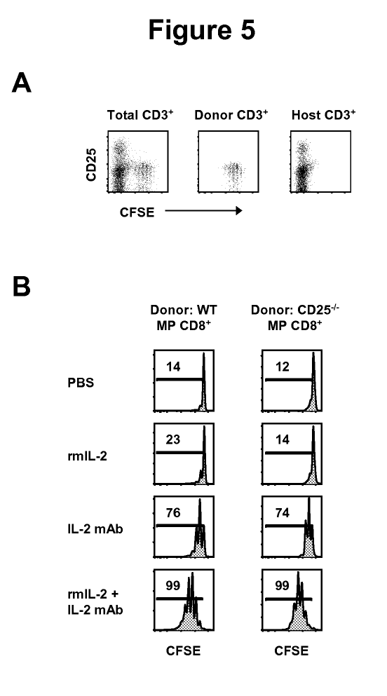

An unexpected finding was that stimulation of MP CD8⁺ cells by IL-2 mAb on adoptive transfer was abolished in IL-2^{-/-} hosts and considerably reduced in IL-2^{-/-} hosts (FIG. 1B). The implication therefore is that, despite its reported neutralizing function in vitro, S4B6 IL-2 mAb functions in vivo by increasing the biological activity of pre-existing IL-2, perhaps through formation of immune complexes. Zurawski et al., J Immunol 137: 3354, 1986. To assess this possibility, we used a regime of daily injections of IL-2 and IL-2 mAb. The resulting proliferation of adoptively-transferred and host MP CD8⁺ cells was dramatically enhanced over that seen with single IL-2 or IL-2 mAb administration (FIG. 3A) and led to a massive (>100-fold) increase in total numbers of MP CD8⁺ cells in spleen and LN on day 7 with marked enlargement of these organs (FIG. 3B). The combined regime of IL-2 and IL-2 mAb also caused a marked (20-30-fold) increase in total numbers of another CD122^{hi} population, namely NK (CD3⁻ NK1.1⁺ DX5⁺) cells, but had minimal effects on other cells, including MP CD44^{hi} CD4⁺ cells and B220⁺ B cells (FIG. 3A, 3B). Proliferation of transferred naïve CD8⁺ cells was relatively low, suggesting that the IL-2/IL-2 mAb combination was acting largely on pre-existing CD122^{hi} cells, rather than on naïve CD122^{lo} precursors (FIG. 4A). Proliferation was independent of IL-15 because comparable data occurred with transfer to IL-15^{-/-} hosts (FIG. 4B). There was also strong stimulation of primed virus-specific CD8⁺ cells (FIG. 3C), indicating that proliferation of CD122^{hi} CD8⁺ cells applied to defined antigen (Ag)-specific memory cells as well as to MP cells. For the latter, proliferation did not lead to CD25 upregulation and was unimpaired with CD25^{-/-} MP CD8⁺ cells, indicating that stimulation occurred only via IL-2Rβγ (CD122) and not IL-2Rαβγ (FIG. 5).

FIG. 3 shows marked selective expansion of MP and antigen (Ag)-specific memory CD8⁺ T cells in vivo by a combination of IL-2 and IL-2 mAb. (A) CFSE-labeled MP CD8⁺T cells were transferred to B6 mice, followed by daily ip injections of PBS, rmIL-2, S4B6 IL-2 mAb or rmIL-2 plus IL-2 mAb. Donor and host cells from lymph nodes (LN) were examined for the markers shown on day 7. Comparable results were obtained for spleen cells. (B) Total spleen and LN cell numbers of donor and host CD44^{hi} T cells from mice in (A) (+SD, 2 mice/group). A photograph of two representative spleens and LN from the injected mice is shown at the right. (C) CFSE-labeled Ag (LCMV)-specific memory CD8⁺ T cells were transferred to B6 mice, followed by daily injections as described above. Donor cells were analyzed on day 7 by flow cytometry. Numbers indicate percentages of divided (CFSE^{lo}) cells (A left column, C).

FIG. 4 shows proliferation of CD8⁺ T cells to IL-2/IL-2 mAb complexes is largely confined to CD122^{hi} MP cells and is IL-15-independent. CFSE-labeled purified MP CD8⁺T cells (A left column, and B) or naïve CD44^{lo} CD8⁺T cells (A right column) were prepared and transferred to WT mice (A) or to IL-15^{-/-} mice (B). Then, daily injections of PBS, 1.5 μg rmIL-2, 50 μg S4B6 IL-2 mAb or 1.5 μg rmIL-2 plus 50 μg IL-2 mAb were administered ip. Donor cells were analyzed on day 7 by flow cytometry. Numbers indicate the percentages of divided (CFSE^{lo}) cells.

FIG. 5 shows proliferation of MP CD8⁺ cells to IL-2/IL-2 mAb complexes in vivo does not require CD25. (A) Purified Thy1.1-marked MP CD122^{hi} CD44^{hi} CD8⁺ T cells from WT B6 mice were labeled with CFSE and transferred to normal B6 (Thy1.2) mice, which then received ip injections of 1.5 μg rmIL-2 plus 50 μg S4B6 IL-2 mAb every other day. On day 7 after adoptive transfer, LN and spleen cells were analyzed by flow cytometry for the expression of CD25 on total CD3⁺ (left), donor Thy1.1⁺CD3⁺ (middle), and host Thy1.1⁻ CD3⁺ cells (right). (B) Purified Thy1.2-marked MP CD8⁺T cells from WT (left column) or CD25^{-/-} (right column) mice were labeled with CFSE and transferred to Thy1.1-marked normal B6.PL mice, which were then injected ip with PBS, 1.5 μg rmIL-2, 50 μg S4B6 IL-2 mAb or 1.5 μg rmIL-2 plus 50 μg IL-2 mAb every other day. 7 d after adoptive transfer, LN and spleen cells were analyzed by flow cytometry. Numbers indicate the percentages of divided (CFSE^{lo}) cells.

### Example 3

### Proliferation of T Cell Subsets in Response to Cytokine Antibody Complex Specific to Monoclonal Antibody

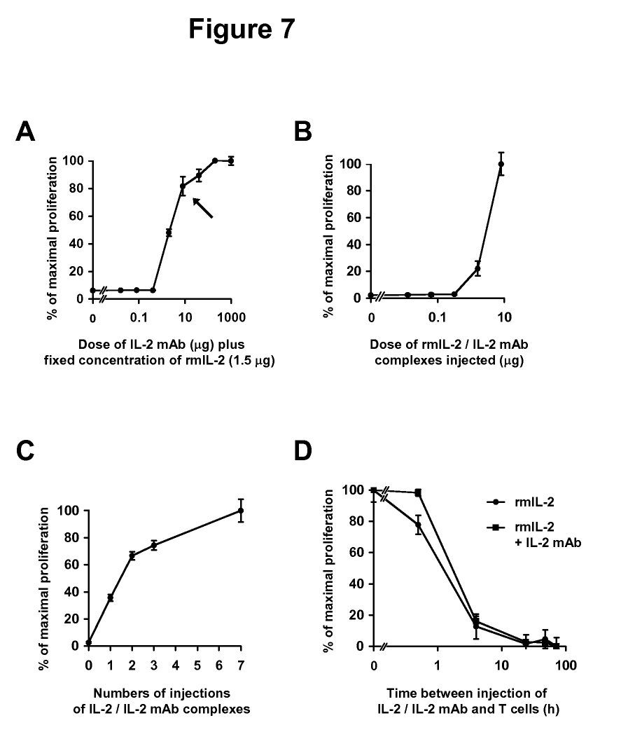

Near-optimal expansion of CD122^{hi} CD8⁺ cells occurred with daily injections of a pre-mixed 2:1 molar ratio of IL-2 to IL-2 mAb for 1 week (FIG. 7A arrow, FIG. 7B). At this ratio, even a single injection of IL-2/IL-2 mAb complex caused considerable expansion of CD122^{hi} CD8⁺ cells (FIG. 7C). Based on the results of injecting IL-2/IL-2 mAb complexes at various times before T cell transfer, the biological half-life of IL-2/IL-2 mAb complexes was determined to be relatively short, i.e. <4 hours (FIG. 7D).

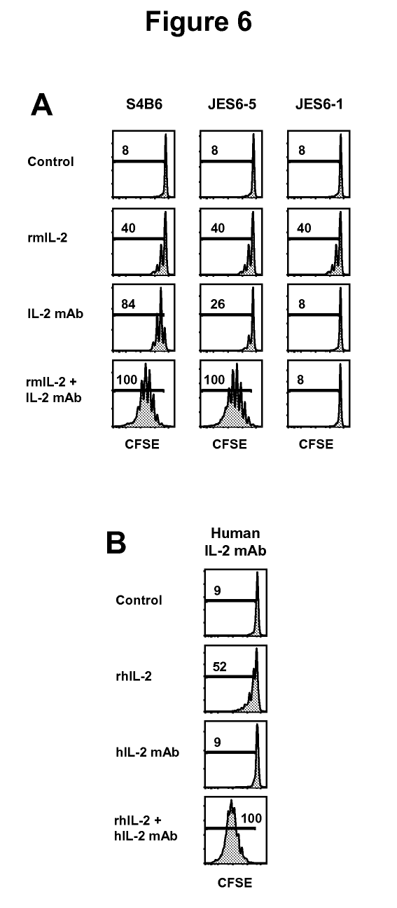

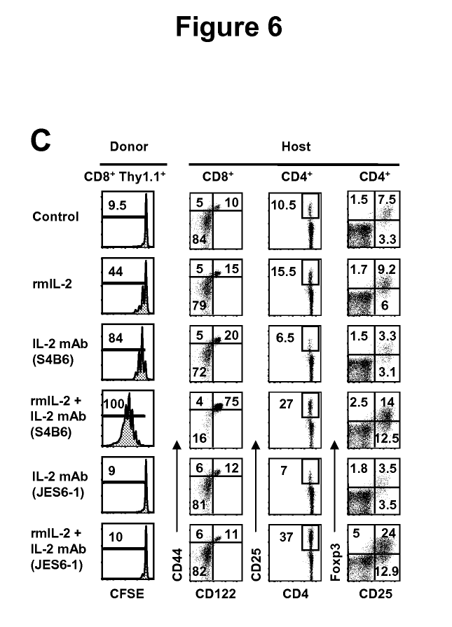

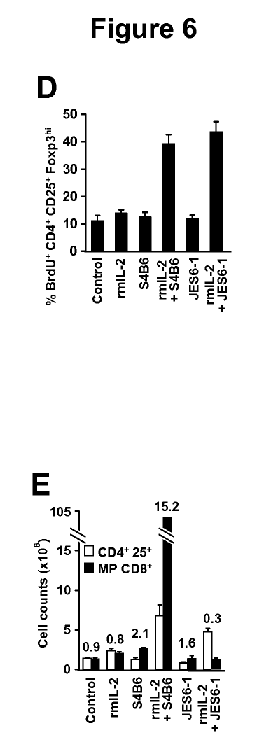

In addition to S4B6 IL-2 mAb, we also observed equivalent proliferation with another anti-mouse IL-2 mAb, JES6-5H4 (JES6-5), plus rmIL-2 (FIG. 6A) and an anti-human IL-2 mAb, MAB602, plus recombinant human IL-2 (rhIL-2) (FIG. 6B). When complexed with IL-2, each of these three mAbs (S4B6, JES6-5 and MAB602) caused marked expansion of CD122^{hi} CD8⁺ cells on adoptive transfer (FIG. 6A, 6B) and strong and selective expansion of host CD122^{hi} cells, including both MP CD8⁺ cells and NK cells.

Interestingly, the results with a third anti-mouse IL-2 mAb, JES6-1A12 (JES6-1), were quite different (FIG. 6A). IL-2/JES6-1 complexes caused lower proliferation of CD122^{hi} CD8⁺ cells than IL-2 alone, indicating that JES6-1 blocked the in vivo response to IL-2. However, JES6-1 plus IL-2 injection led to mild proliferation of a different IL-2-responsive population, namely CD25⁺CD4⁺ cells (FIG. 6C, D). These cells were predominantly Foxp3⁺ and thus resembled T regs. Expansion of these cells was also seen with injection of the other IL-2 mAbs, although this effect was dwarfed by the huge expansion of CD122^{hi} CD8⁺ cells (FIG. 6E).

FIG. 6 shows selective stimulation of T cell subsets by different IL-2/IL-2 mAb complexes. CFSE-labeled MP CD8⁺ T cells were transferred to B6 mice, followed by (A, B) daily ip injections of control mAb, IL-2 (rmIL-2 in A, rhIL-2 in B), IL-2 mAb or IL-2 plus IL-2 mAb as in FIG. 3A. The IL-2 mAbs used were (A) anti-mouse S4B6, JES6-5 or JES6-1, or (B) anti-human MAB602. (C) MP CD8⁺ T cells were transferred to B6 mice, followed by daily injections of control mAb, rmIL-2, IL-2 mAb, or rmIL-2 plus IL-2 mAb as above. Donor and host cells from spleen were examined for the markers shown on day 7. (D) Mice treated as in (C) were given BrdU in the drinking water for the last 3 days. Shown are the percentages of CD3⁺ CD4⁺ CD25⁺ Foxp3^{hi} cells that were BrdU⁺ (+SD, 2 mice/group). (E) Total cell counts of CD4⁺ CD25⁺ and MP CD8⁺ cells in spleen from mice in (C). Numbers on top of the bars indicate the ratios of MP CD8⁺ to CD4⁺ CD25⁺ cells. Mice were analyzed on day 7 (A-E). Numbers indicate percentages of divided (CFSE^{lo}) cells (A, B, C left column).

FIG. 7 shows requirements for stimulating MP CD8⁺ cells with IL-2/IL-2 mAb complexes in vivo. (A) Purified Thy1.1-marked MP CD8⁺ T cells were labeled with CFSE and transferred to B6 mice, which then received daily injections of titrated doses (0 to 1000 μg) of S4B6 IL-2 mAb plus a fixed concentration (1.5 μg) of rmIL-2. On day 7 after adoptive transfer, LN and spleen cells were analyzed by flow cytometry. The arrow denotes proliferation observed with injection of a 2:1 molar ratio of IL-2 to IL-2 mAb, i.e. where neither reagent was in excess. (B) Recipients of purified CFSE-labeled MP CD8⁺ cells were given daily titrated doses of a 2:1 molar ratio of rmIL-2/S4B6 IL-2 mAb complexes, starting at 1.5 μg rmIL-2 plus 8 μg S4B6 IL-2 mAb and titrating down in 5-fold dilutions. On day 7 after adoptive transfer, LN and spleen cells were analyzed by flow cytometry. (C) Recipients of purified CFSE-labeled MP CD8⁺ cells were given ip injections of 1.5 μg rmIL-2 plus 8 μg S4B6 IL-2 mAb at various time points after adoptive transfer: 0, no rmIL-2 plus S4B6; 1, rmIL-2 plus S4B6 on day 0; 2, rmIL-2 plus S4B6 on days 0 and 4; 3, rmIL-2 plus S4B6 on days 0, 2 and 4; 7, daily injections of rmIL-2 plus S4B6. All mice were sacrificed on day 7 after adoptive transfer and LN and spleen cells were analyzed by flow cytometry. (D) Recipients of purified CFSE-labeled MP CD8⁺ cells were given one single injection of 1.5 μg rmIL-2 () or 1.5 μg rmIL-2 plus 50 μg S4B6 IL-2 mAb (▪) at 72 h, 48 h, 24 h, 4 h or 30 min. before adoptive transfer of T cells, or at the same time as the adoptive cell transfer for time point 0. All data in this figure are expressed as percent of maximal proliferation where the maximal proliferation was set as 100% and the other values were calculated relative to it.

### Example 4

### Monoclonal Antibodies that May Bind to Different Sites on IL-2

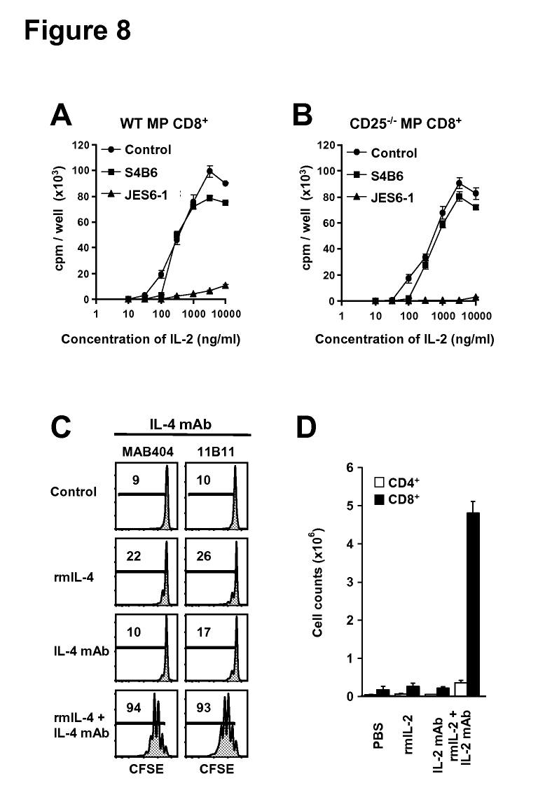

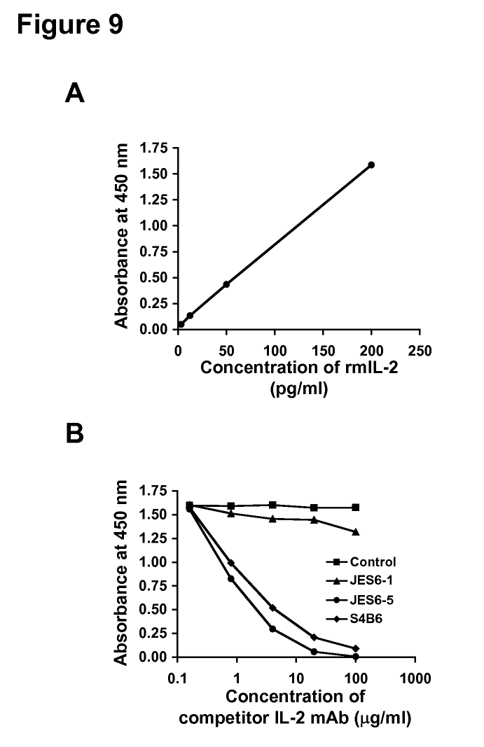

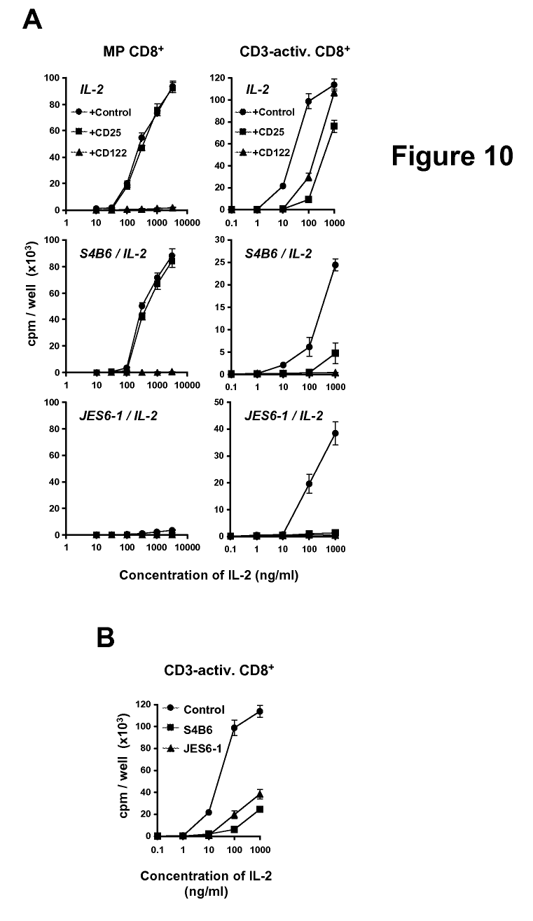

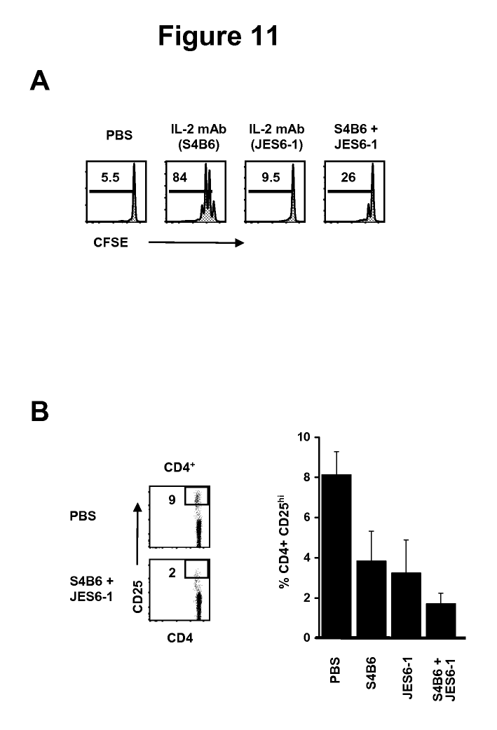

The above results suggested that S4B6 and related mAbs may bind to a different site on IL-2 than JES6-1. IL-2/IL-2 mAb sandwich ELISA assays provided direct support for this possibility (FIG. 9). As in vivo, JES6-1 totally blocked the response of both normal and CD25^{-/-} MP CD8⁺ cells to IL-2 in vitro via CD122 (IL-2Rβγ) (FIG. 8A, 8B). However, as for CD4⁺ CD25⁺ T regs in vivo, JES6-1/IL-2 complexes were able to induce weak but significant in vitro stimulation of cells expressing high-affinity IL-2Rαβγ, namely CD25⁺ CD3-activated naïve CD8⁺ cells (FIG. 10); these cells were very sensitive to IL-2 and easily inhibited by CD25 mAb. Thus, JES6-1 mAb apparently binds to an IL-2 site that is crucial for interaction with CD122 but less crucial for binding to CD25 (IL-2Rαβγ). In contrast, S4B6 failed to inhibit (or enhance) the response of MP CD8⁺ cells to IL-2 in vitro (FIG. 8A, 8B) but strongly inhibited the IL-2 response of CD3-activated CD8⁺ cells (FIG. 10). Hence, S4B6 binds to an IL-2 site that partly occludes binding to CD25 but does not impede binding to CD122. Notably, when not complexed with exogenous IL-2, a mixture of JES6-1 and S4B6 mAbs caused near abolition of T cell proliferation in vivo, both for MP CD8⁺ cells and for T regs (FIG. 11), further suggesting that S4B6 and JES6-1 recognize different sites on IL-2.

FIG. 8 shows features of T cell stimulation by cytokine/mAb complexes. (A, B) Purified MP CD8⁺ cells from WT (A) or CD25^{-/-} (B) mice were cultured together with titrated concentrations of a 2:1 molar ratio of rmIL-2 plus S4B6 mAb (S4B6, ▪), or rmIL-2 plus JES6-1 mAb (JES6-1, ▴) for 3 days; soluble IL-2 plus an irrelevant mAb was used as a control (Control, ). Proliferation was measured by adding [³H]-thymidine for the last 16 h. (C) Purified CFSE-labeled MP CD8⁺ cells were transferred to B6 mice, which were then given every other day ip injections of control mAb, rmIL-4, IL-4 mAb (MAB404 or 11B11) or rmIL-4 plus IL-4 mAb. Mice were analyzed on day 7. Numbers indicate percentages of divided (CFSE^{lo}) cells. (D) B6 mice were irradiated with 1000 cGy and injected iv with unseparated vs T-depleted B6 BM cells, followed by daily ip injections of PBS, rmIL-2, S4B6 IL-2 mAb or mL-2 plus S4B6 IL-2 mAb. 8 days after adoptive transfer spleen cells were analyzed by flow cytometry. Shown are mean cell numbers of CD3⁺ CD4⁺ and CD3⁺ CD8⁺ cells from recipients of unseparated BM (+SD, 2 mice/group). With injection of T-depleted BM, no restoration of T cell numbers occurred.

FIG. 9 shows JES6-5 and S4B6 IL-2 mAb bind to similar sites on IL-2 which are distinct from the binding site of JES6-1. (A) A standard IL-2 sandwich ELISA with plate-bound unconjugated JES6-1 as capture mAb and biotinylated JES6-5 as detection mAb was used for detecting titrated concentrations of rmIL-2, starting at 200 pg/ml and titrating down in 4-fold dilutions. Binding of the detection mAb was quantitated using streptavidin-conjugated horseradish peroxidase together with the substrate o-phenylenediamine, and absorbance was measured at 450 nm (see Materials and Methods). (B) The ELISA approach in (A) was modified by using JES6-1 as a capture mAb plus a fixed concentration (200 pg/ml) of rmIL-2. Then, purified unconjugated competitor mAbs were added at titrated concentrations (5-fold dilutions starting at 100 μg/ml) to the wells to detect whether these mAbs could block binding of biotinylated JES6-5 detection mAb. The competitor mAbs used were control mAb, JES6-1, JES6-5, or S4B6 IL-2 mAb. The samples were then measured as described above.

FIG. 10 shows effects of IL-2/IL-2 mAb complexes in vitro. Purified MP CD8⁺ cells from B6 mice (A left column), or total CD8⁺ cells activated by plate-bound anti-CD3 mAb (CD3-active. CD8⁺; A right column, and B), were cultured together with titrated concentrations of a 2:1 molar ratio of rmIL-2 plus S4B6 mAb (S4B6/IL-2, middle row), or rmIL-2 plus JES6-1 mAb (JES6-1/IL-2, bottom) for 3 days; soluble IL-2 plus an irrelevant mAb was used as a control (IL-2, top). A fixed concentration (10 μg/ml) of an irrelevant control mAb (+Control, ), CD25 mAb (+CD25, ▪), or CD122 mAb (+CD122, ▴) was also added to the wells. (B) CD3-active. CD8⁺ cells, prepared as in (A), were cultured together with titrated concentrations of a 2:1 molar ratio of rmIL-2 plus S4B6 mAb (S4B6, ▪), or rmIL-2 plus JES6-1 mAb (JES6-1, ▴) for 3 days; soluble IL-2 plus an irrelevant mAb was used as a control (Control, ). Proliferation was measured by adding [³H]-thymidine for the last 16 h.

FIG. 11 shows injecting a mixture of S4B6 and JES6-1 IL-2 mAbs blocks proliferation of both MP CD8⁺ cells and CD4⁺ CD25⁺ cells. (A) Purified Thy1.1-marked MP CD122^{hi} CD44^{hi} CD8⁺T cells from WT B6 mice were labeled with CFSE and transferred to normal B6 (Thy1.2) mice, which then were injected ip with PBS, 50 μg S4B6 IL-2 mAb, 50 μg JES6-1 IL-2 mAb, or a combination of 50 μg S4B6 IL-2 mAb together with 50 μg JES6-1 IL-2 mAb every day. On day 7 after adoptive transfer, LN and spleen cells were analyzed by flow cytometry. Numbers indicate the percentages of divided (CFSE^{lo}) cells. (B) Mice treated as in (A) were sacrificed after 7 days, and (endogenous) CD4⁺ CD25⁺ cells analyzed by flow cytometry (left) and quantified (right). Numbers indicate the percentages of CD4⁺ CD25⁺ cells in the quadrant. The data at the right refer to the percent of CD4⁺ cells that were CD25^{hi}.

### Example 5

### Complexes of IL-2 Plus F(ab′)₂ Monoclonal Antibody Fragments are Much Less Stimulatory than Complexes of IL-2 Plus Intact Monoclonal Antibody

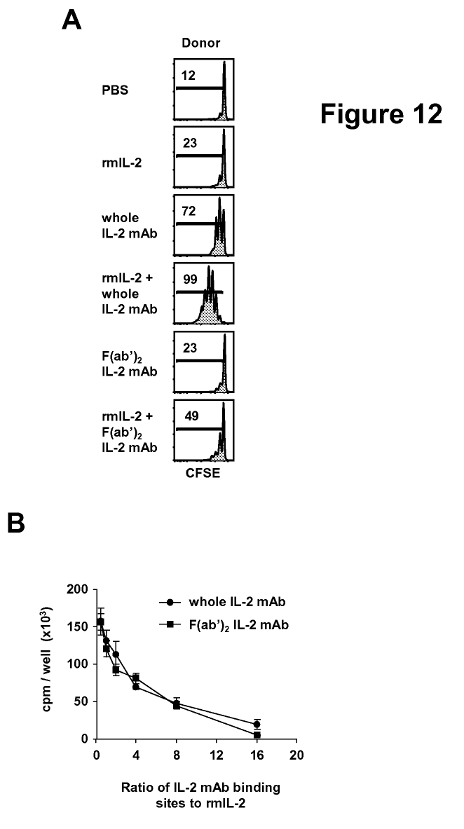

Why IL-2/IL-2 mAb complexes are so potent in vivo is unclear. It was reported previously that binding to antibody can increase the half-life of IL-2, and also IL-4, in vivo but, other than inducing a mild increase in NK cell-mediated tumor rejection, the effects of IL-2/IL-2 mAb complexes on T cells were not mentioned. Sato et al., Biotherapy 6: 225, 1993; Finkelman et al., J Immunol 151: 1235, 1993; Courtney et al., Immunopharmacology 28: 223, 1994. It was also reported that removal of the Fc portion of anti-IL-2 mAb did not alter the increased half-life of IL-2 and did not diminish the increased NK cell-mediated anti-tumor activity. Sato et al., Biol Pharm Bull 17:1101, 1994. In contrast, for the marked expansion of CD122^{hi} MP CD8⁺ cells reported here, F(ab′)₂ mAb fragments were much less stimulatory than intact mAb (FIG. 12), suggesting that the complexes became bound to cells via the mAb Fc region. Such presentation may be unusually efficient and explain why IL-2/IL-2 mAb complexes are far more stimulatory in vivo than in vitro.

FIG. 12 shows F(ab′)₂ fragments of IL-2 mAb are much less efficient than whole IL-2 mAb. (A) Purified Thy1.1-marked MP CD122^{hi} CD44^{hi} CD8⁺ T cells from B6 mice were labeled with CFSE and transferred to normal B6 (Thy1.2) mice, which then received ip injections every other day of PBS, 1.5 μg rmIL-2, 50 μg whole S4B6 IL-2 mAb, 1.5 μg rmIL-2 plus 50 μg whole S4B6 IL-2 mAb, 50 μg F(ab′)₂ S4B6 IL-2 mAb, or 1.5 μg rmIL-2 plus 50 μg F(ab′)₂ S4B6 IL-2 mAb. After 7 days, LN and spleen cells were analyzed by flow cytometry. Numbers indicate the percentages of divided (CFSE^{lo}) cells. (B) F(ab′)₂ S4B6 IL-2 mAb was compared to whole S4B6 IL-2 mAb in vitro for its ability to inhibit rmIL-2 driven proliferation of the IL-2-sensitive cell line CTLL-2. CTLL-2 cells, 2×10⁴ cells/well, were cultured for 48 h in the presence of a fixed concentration (100 ng/ml) of rmIL-2 and titrated doses of either F(ab′)₂ S4B6 IL-2 mAb (▪) or whole S4B6 IL-2 mAb (). Shown is the ratio of IL-2 mAb binding sites to rmIL-2 molecules. 1 μg of F(ab′)₂ S4B6 IL-2 mAb (MW ˜100 kD) equals 1.5 μg of whole S4B6 IL-2 mAb (MW ˜150 kD) in terms of IL-2 binding sites. Proliferation was measured by adding [³H]-thymidine for the last 24 h of the culture period.

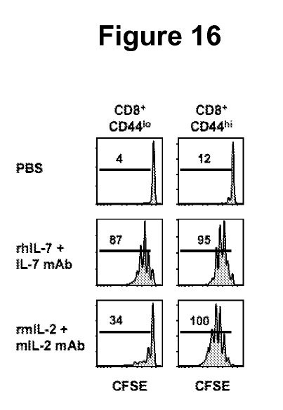

The stimulatory effects of IL-2/IL-2 mAb complexes also applied to complexes of IL-4 and IL-4 mAb (FIG. 8C) and IL-7 and IL-7 mAb (FIG. 16). Thus, proliferation of CD8⁺ cells was much higher after injection of these cytokine/mAb complexes than with cytokine or mAb alone.

For S4B6 and related antibodies, injecting IL-2/IL-2 mAb complexes might be clinically useful for tumor immunotherapy and for expanding T cell numbers after bone marrow (BM) transplantation. In support of this latter idea, irradiated mice given unseparated BM cells and then a course of IL-2/S4B6 injections showed a rapid restoration of mature T cell numbers, especially CD8⁺ cells, as early as 1 week post-transfer (FIG. 8D). Conversely, expansion of CD4 T regs by IL-2 and JES6-1 or related mAbs could be useful for treating autoimmune disease.

### Example 6

### IL-2/IL-2 mAb Complexes Display Significantly Higher Biological Activity than Covalently Linked IL-2-Ab Recombinant Fusion Proteins

Upon their introduction in the early 90s, the cytokine/mAb complexes received only brief attention thereafter, presumably because of concomitant advent of recombinant fusion proteins comprised of cytokines covalently linked to Abs (Davis and Gillies, Cancer Immunol Immunother 52:297-308, 2003; Cruz et al., Clin Exp Med 4:57-64, 2004). Since the increased biological activity of cytokine/mAb complexes was largely considered to reflect the increased half-life of the cytokine, the recombinant fusion proteins were favored because of their convenience and versatility in production. Thus, fusion proteins were constructed whereby IL-2, GM-CSF or IL-12 were covalently linked to either an anti-hapten mAb, to promote prolonged in vivo longevity, or to a mAb reactive to tumor antigens, thus directing the cytokines to tumor sites (Davis and Gillies, Cancer Immunol Immunother 52:297-308, 2003; Cruz et al., Clin Exp Med 4:57-64, 2004; Lode et al., Pharmacol Ther 80:277-292, 1998). At least two Ab-IL-2 fusion proteins were generated and studied over the past 13 years (Davis and Gillies, Cancer Immunol Immunother 52:297-308, 2003; Cruz et al., Clin Exp Med 4:57-64, 2004; Lode et al., Pharmacol Ther 80:277-292, 1998). Studies in mice showed that these fusion proteins displayed significantly better tumoricidal activity than either the Ab or cytokine alone or as a mixture without the covalent linkage (Davis and Gillies, Cancer Immunol Immunother 52:297-308, 2003; Cruz et al., Clin Exp Med 4:57-64, 2004; Lode et al., Pharmacol Ther 80:277-292, 1998). However, other than showing that tumoricidal activity required CD8 cells and NK cells, the direct in vivo effects of the fusion proteins on T cell subsets were largely ignored.

Although one might expect the biological activity of the fusion proteins to be identical to that of cytokine/mAb complexes under in vivo conditions, these constructs have yet to be compared directly. To this end, we have recently obtained Ab-IL-2 fusion proteins from Dr. Sherie Morrison (UCLA, CA) for comparison with IL-2/mAb complexes. The recombinant fusion protein, designated anti-DNS-IgG3-IL-2, is comprised of human IL-2 covalently attached to the Fc end of a chimeric Ab, containing human IgG3 constant region with mouse variable region specific for an hapten, dansyl (5-dimethylamino naphthalene 1-sulfonyl chloride, DNS) (Harvill et al., J Immunol 157:3165-3170, 1996). Since DNS is not found in the mouse, this fusion protein should persist systemically, similar to cytokine/mAb complexes. The in vivo half-life of anti-DNS-IgG3-IL-2 fusion protein in mice is measured to be ˜7 hr (Harvill et al., J Immunol 157:3165-3170, 1996), which is similar to that of IL-2/mAb complexes (Sato et al., Biotherapy 6:225-231, 1993) and slightly longer than of the other Ab-IL-2 fusion protein ch14.18-IL-2 (Kendra et al., Cancer Immunol Immunother 48:219-229, 1999). Under in vitro conditions all these reagents displayed IL-2 activity similar to free rIL-2 (Gillies et al., Proc Natl Acad Sci USA 89:1428-32; Harvill and Morrison, Mol Immunol 33:1007-1014, 1996).

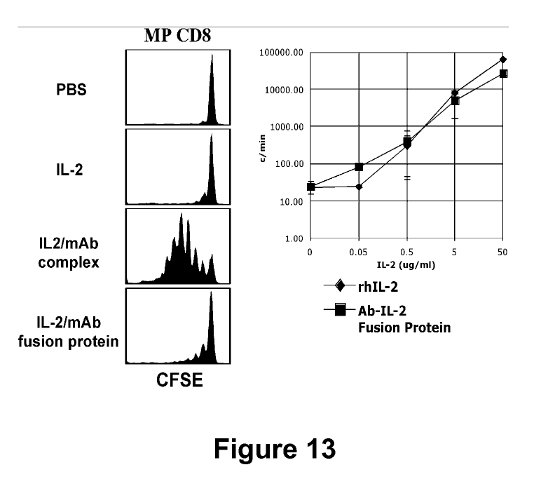

To directly compare the biological activity of anti-DNS-IgG3-IL-2 fusion proteins to IL-2/IL-2 mAb complexes, CFSE-labeled MP CD8 cells from B6.PL mice were purified, injected into a group of unirradiated B6 mice, which were then injected with either PBS, rhIL-2, the fusion protein or the complex at a molar equivalent dose. Since the fusion protein was constructed with human IL-2, the complex was created by binding rhIL-2 with anti-human IL-2 mAb (MAB602), which we showed to be very effective in inducing proliferation of mouse MP CD8 cells (FIG. 6B). Strikingly, unlike the efficient donor cell proliferation induced by control rhIL-2/MAB602 complexes, anti-DNS-IgG3-IL-2 fusion proteins, which displayed expected IL-2 activity, were minimally effective in promoting the donor cell proliferation; in fact the fusion proteins were no better than free rhIL-2 (FIG. 13). This finding strongly indicates that IL-2/mAb complexes display significantly higher in vivo biological activity than analogous fusion proteins.

FIG. 13 shows IL-2/mAb complexes are significantly more potent than Ab-IL-2 fusion proteins. CFSE-labeled Thy-1.1 memory-phenotype (MP) CD8 cells were transferred into normal B6 mice and then injected with either PBS, 1 μg rhIL-2, 1 μg rhIL-2+5 MAB602 or a molar equivalent (˜10 μg) of anti-DNS-IgG3-IL-2 fusion proteins every other day and then CFSE profiles of donor CD8 cells in host LN analyzed 7 d after start of the experiment (left side). IL-2 activity was measured by incubating CTLL-2 cells with molar equivalent titrating concentrations of rhIL-2 or anti-DNS-IgG3-IL-2 fusion proteins (right side).

### Example 7

### Ability of IL-7/IL-7 Monoclonal Antibody Complex to Expand Naïve T Cells

IL-7 is a small (MW: ˜25 Kd) type-I cytokine that belongs to same family of cytokines as IL-2, -4, -9, -15 and -21 (Fry and Mackall, J Immunol 174:6571-76, 2005; Sugamura et al., Annu Rev Immunol 14:179-205. 1996). IL-7 was initially discovered in 1988 for its ability to support growth of B cell progenitors, and the gene was cloned from a bone-marrow (BM) stromal cell line (Namen et al., Nature 333:571-73, 1988; Namen et al., J Exp Med 167:988-1002, 1988). The T cell-tropic function of IL-7 was subsequently realized, starting with the finding that IL-7 promotes growth and differentiation of T cell progenitors for both αβ and γδ TCR subsets in the thymus (Conlon et al., Blood 74:1368-73, 1989; Watanbe et al., Int Immunol 3:1067-75, 1991), and with the confirmation of these roles with the generation of IL-7- and IL-7 receptor (R)-deficient mice (Peschon et al., J Exp Med 180:1955-60, 1994; vonFreeden-Jeffry et al., J Exp Med 181:1519-26, 1995). Severe impairment in both B and T cell development in these mutant mice demonstrated a non-redundant role for IL-7 in B and T lymphopoiesis. Nonetheless, variation in the dependence of IL-7 between different species is apparent, as immunodeficient human patients with defective IL-7R are severely deficient in T cells, but possess normal numbers of B cells (vonFreeden-Jeffry et al., J Immunol 161:5673-5680, 1998; Puel et al., Nat Genet 20:394-97, 1998; vonFreeden-Jeffry et al., Immunity 7:147-154, 1997; Schluns et al., Nature Immunol 1:426-32, 2000; Tan et al., Proc Natl Acad Sci USA 98:8732-37, 2001).

The overall size and composition of the mature T cell pool are regulated by homeostatic mechanisms. Surh et al., Sem. Immunol. 17:183, 2005; Schluns et al., Nat Rev Immunol. 3:269, 2003; Jameson Nat Rev Immunol. 2:547, 2002. Survival of a constant number naïve T cells require signals from contact with self-MHC/peptide ligands and IL-7, whereas signals from contact with IL-7 and IL-15 are required for survival of constant number of memory T cells. Surh et al., Sem. Immunol. 17:183, 2005; Schluns et al., Nat Rev Immunol. 3:269, 2003; Jameson Nat Rev Immunol. 2:547, 2002. The presence of homeostatic mechanisms that regulate the overall size of the T cell pool is apparent by the finding that T cells have the capacity to undergo spontaneous “homeostatic” expansion in a response to T cell (T) deficiency. Ernst et al., Immunity 11:173, 1999; Goldrath et al., Immunity 11:183, 1999. Moreover, homeostatic expansion of T cells is dependent on IL-7 and IL-15. Thus, in the absence of IL-7 homeostatic expansion of naïve T cells fails to occur and in the absence of both IL-7 and IL-15 memory T cells cannot undergo homeostatic expansion. Schluns et al., Nat Immunol 1:426, 2000; Tan et al., Proc Natl Acad Sci 98:8732, 2001; Tan et al., J Exp Med 195:1523, 2002; Goldrath et al., J Exp Med 195: 1515, 2002. These findings collectively have led to the current paradigm that constitutively produced basal levels of IL-7 and IL-15 supports survival of a finite number of T cells, and upon T cell depletion, the basal concentrations of IL-7 and IL-15 increase, from lack of utilization, and drive the remaining T cells to undergo homeostatic expansion Surh et al., Sem. Immunol. 17:183, 2005. It should be emphasized that survival and homeostatic expansion of naïve T cells are almost exclusively dependent on IL-7, whereas survival and homeostatic proliferation of memory T cells can be supported by either IL-7 or IL-15, but most optimally by both IL-7 and IL-15. Surh et al., Sem. Immunol. 17:183, 2005; Schluns et al., Nat Rev Immunol. 3:269, 2003; Jameson Nat Rev Immunol. 2:547, 2002.

To be effective in vivo, IL-7 has to be injected in large amounts. For instance, injecting IL-7 in quantities sufficient to raise blood levels 10-100 fold for 3 wks caused only a 3-7-fold increase in T cell numbers in humans [Rosenberg, 2006 #1889]. Hence much of the administered IL-7 may have limited biological activity, perhaps because of a short half life or a failure to reach appropriate sites in the lymphoid tissues. With regard to the former, it was shown several years ago that the half-life of several γ_{c} cytokines, including IL-7, can be increased by binding to specific anti-cytokine mAbs [Sato, 1993 #1805; Courtney, 1994 #1895; Finkelman, 1993 #1775; Valenzona, 1998 #1896; Klein, 1995 #1876]. For IL-2, however, we have recently shown that association with IL-2 mAb has a much more dramatic effect on cytokine activity in vivo than can be attributed solely from an increase in cytokine half-life [Boyman, 2006 #1835; Kamimura, 2006 #1840]. In this report we show that IL-7/IL-7 mAb complexes are vastly superior to free IL-7 in eliciting expansion of pre-B cells, and pre-T cells. These complexes also act strongly on mature CD4⁺ and CD8⁺ T cells and cause both naïve and memory T cells to undergo efficient homeostatic expansion under normal T cell-replete conditions.

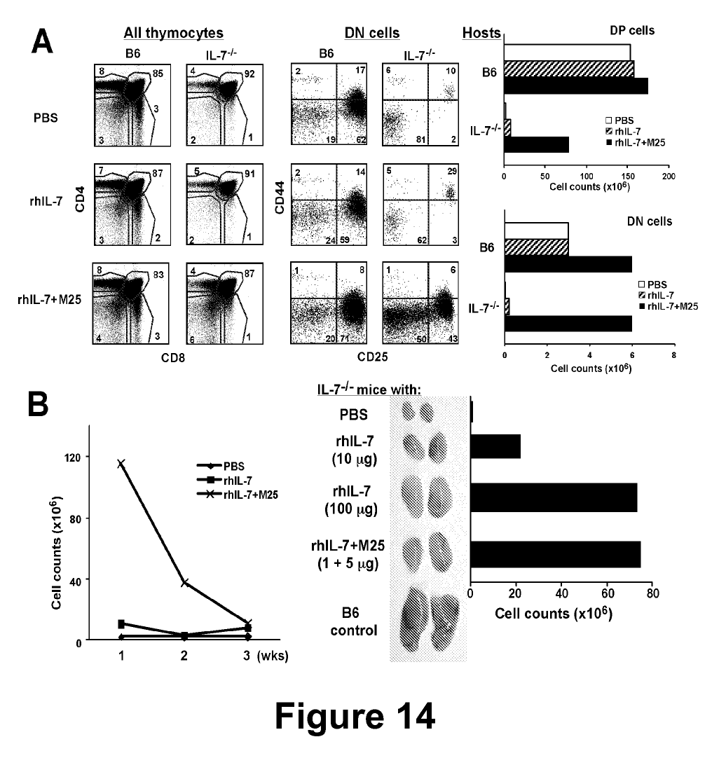

IL-7/IL-7 mAb complexes can augment or restore thymopoiesis. The thymus of B6 mice injected with rhIL-7/IL-7 mAb (M25) complexes had 15-20% higher cellularity than PBS-injected B6 mice, mostly from a rise in numbers of CD4/CD8 double positive (DP) cells (FIG. 14). To better assess the effect on thymopoiesis, groups of IL-7^{-/-} mice, which have a very small thymus (10, 11), were injected with rhIL-7/M25 complexes, rhIL-7 alone or PBS. Two injections of 1.5 μg rhIL-7 plus 15 μg M25, 3 d apart, caused the thymus in IL-7^{-/-} mice to greatly enlarge, and showed a 50-100-fold increase in cellularity by 7 d; by contrast injection of 1.5 μg rhIL-7 alone induced only a relatively minor 2-3-fold increase in cell number (FIG. 14). Analysis of the CD4−CD8− (DN) population of thymocytes revealed that injection of rhIL-7/M25 complexes induced the selective emergence of CD25+CD44− DN3 and CD25−CD44− DN4 cells, which were severely deficient in IL-7^{-/-} mice; these cells were not restored with injection of 1.5 μg rhIL-7 alone (FIG. 14A). The restoration of thymopoiesis induced by rhIL-7/M25 complexes was transient as the thymus of the injected IL-7^{-/-} mice reverted to a hypocellular state by 3 wks after injecting the complexes (FIG. 14B, left). It should be mentioned that in contrast to the marked effect on the thymus, injection of rhIL-7/M25 complexes caused only a 2-fold of increase in the spleen cellularity of IL-7^{-/-} mice.

To estimate the relative biological activity of rhIL-7/M25 complexes, IL-7^{-/-} mice were injected twice over 7 d with a moderate dose (1+5 μg) of rhIL-7/M25 complexes vs. a titrated doses (1, 10 and 100 μg) of free rhIL-7. The striking finding was that enlargement of the thymus induced by 1 μg rhIL-7 bound to M25 was equivalent to the thymus size elicited by injecting 100 μg of free rhIL-7 (FIG. 14B, right).

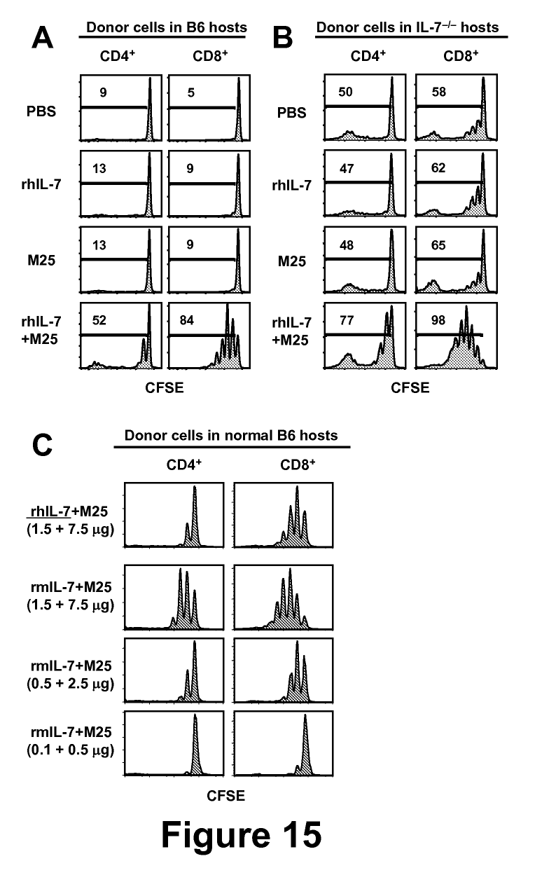

FIGS. 15 through 18 provide further evidence for increased biological activity of IL-7/IL-7 mAb complex over IL-7. FIG. 15 shows IL-7/IL-7 mAb (M25) complexes induce homeostatic proliferation of naïve T cells. To assess the ability of IL-7/M25 complexes to induce expansion of mature T cells, CFSE-labeled CD45-congenic B6.CD45.1 LN cells were adoptively transferred into unirradiated normal B6 mice. These hosts were then injected with rhIL-7/M25 complexes (1.5+7.5 μg, 3× over 7 days) and the fate of donor cells analyzed; control hosts received only PBS, rhIL-7 or M25. Notably, while donor B and T cells did not proliferate in control hosts, injections of rhIL-7/M25 complexes induced up to 4-5 rounds of proliferation of most donor CD8⁺ cells, one round of proliferation of about a one half of the donor CD4⁺ cells, and almost no proliferation of donor B220+B cells ((FIG. 15A). Considering the slow pace of the proliferation, it is likely that the rhIL-7/M25 complexes caused “homeostatic” proliferation of donor T cells in response to self-MHC/peptide ligands, despite being in normal T cell-replete hosts. Consistent with this notion, rhIL-7/M25-induced proliferation two lines of naïve TCR transgenic CD8⁺ cells tested (P14 and OT-1), and proliferation of naïve CD8⁺ cells was largely abrogated in the absence of MHC class I molecules, i.e., in TAP-1^{-/-} hosts. Moreover, injection of rhIL-7/M25 complexes caused naïve T cells to undergo homeostatic proliferation in IL-7^{-/-} hosts, which do not support homeostatic proliferation donor naïve T cells (FIG. 15B). Here, control injection of rhIL-7 alone at the equivalent dose failed to elicit donor T cell proliferation (FIG. 15B). Finally, rhIL-7/M25 induced proliferation of donor T cells by directly engaging IL-7R, as the proliferation was completely abrogated when anti-IL-7Rα mAb A7R34 was co-injected with the complexes.

As for homeostatic proliferation to endogenous IL-7 in lymphopenic hosts (12, 13), injection of rhIL-7/M25 complexes caused much weaker proliferation of CD4+ cells than CD8+ cells. Since mouse (m) IL-7R binds rhIL-7 with a slightly lower affinity than rmIL-7, we examined the effects of rmIL-7/M25 complexes on CD4⁺ cells. Notably, rmIL-7/M25 complexes displayed 2-3-fold greater biological activity than rhIL-7/M25 complexes and, significantly, induced efficient proliferation of both donor CD4⁺ and CD8⁺ subsets of cells in normal B6 hosts (FIG. 15C). Proliferation of both CD4⁺ and CD8⁺ cell subsets was also seen with rhIL-7/M25 complexes when these complexes were injected in higher doses. Expansion also applied for host T cells as the size of the naïve T cell pool in these hosts increased about 3-fold. A massive expansion of B cell precursors was observed in spleen and bone marrow of mice injected with IL-7+M25 complex as previously reported. Finkelman et al., J Immunol 151: 1235, 1993.

FIG. 16 shows that rhIL-7+M25 (IL-7/mAb) complex can drive expansion of both naïve and memory T cells. IL-7/mAb complex is almost as effective as IL-2/mAb complex in expanding memory (CD44^{hi}) CD8 cells, but IL-7/mAb is much more efficient than IL-2/mAb in inducing expansion of naïve (CD44^{lo}) CD8 cells. IL-7/mAb complex was also found to induce homeostatic proliferation of naïve and memory CD4 cells, but at lower rates than CD8 cells (FIG. 15C). In experimental procedures, CFSE-labeled B6.Thy-1.1 naïve (CD44^{lo}) and purified memory (CD44^{hi}) CD8 cells were injected into normal B6 mice and the hosts were then injected with PBS, IL-7+M25 (1.5+15 μg), or IL-2+S4B6 (1.5+15 μg) every other day. Donor T cells were analyzed 7 days after cell injection by flow cytometry after staining host splenic cells for Thy-1.1, and CD8. Shown are CFSE profiles of gated donor CD8 cells. Similar data were obtained from LN.

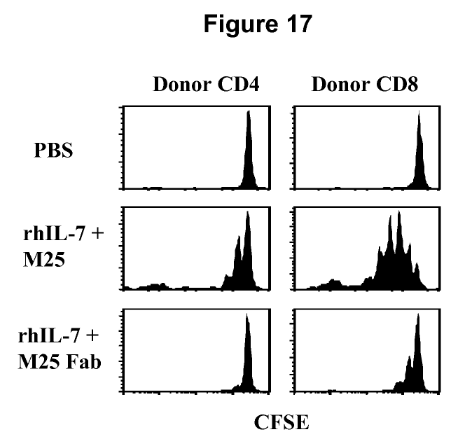

FIG. 17 shows that the Fc portion of anti-IL-7 mAb M25 is required for its enhancing effect when complexed to IL-7. Removal of the Fc portion from M25 destroys most of the capacity of IL-7+M25 complex to induce expansion of naïve T cells. In experimental procedures, CFSE-labeled B6.Thy-1.1 LN cells were injected into normal B6 mice and the hosts were then injected with PBS, IL-7+M25 (1.5+15 μg/injection), or IL-7+M25 Fab (1.5+15 μg/injection) every other day. Donor T cells were analyzed 7 d after cell injection by flow cytometry after staining host LN cells for Thy-1.1, CD4 and CD8. Shown are CFSE profiles of gated donor CD4 and CD8 cells. Similar data were obtained from spleen.

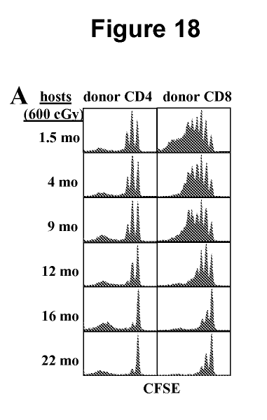

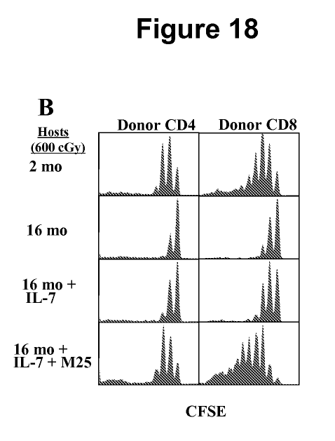

FIG. 18 shows the ability of IL-7/mAb complex to restore defect in naïve T cell homeostasis apparent with advanced age. The mature T cell pool in young individuals is composed of mostly naïve cells. Aging does not significantly change the total number and the ratio of CD4⁺ to CD8⁺ cells, but is associated with a gradual increase in the proportion of memory cells with the compensatory reduction in naïve cells. Hodes Immunol. Rev. 160:5, 1997; Miller Vaccine 18:1654, 2000; Linton et al., Nat. Immunol. 5:133, 2004. Although the exact cause of the age-associated shift in the representation of naïve and memory cells is unknown, the simplest idea is that this is a reflection of decreased thymic output of naïve cells combined with the continued antigen-driven conversion of naïve cells into memory cells throughout life. This view, however, is likely to be an over-simplification as there are probably multiple mechanisms contributing to loss of naïve T cell with aging. One likely contributing factor could be defect in the homeostatic mechanisms that regulate the survival and the overall size of the mature T cell pool. We have recently found evidence that aging is associated with a severe decline in the innate ability to support homeostasis of naïve T cells. This defect appears to be IL-7 related, but it is not due to a decline in production of IL-7. Rather, there seems to be a problem in presentation of IL-7 to T cells. Although the underlying cause of this defect is yet to be identified, we describe a novel method to reverse age-induced decline in the ability to support homeostasis of naïve T cells.

FIG. 18 shows that exogenous free IL-7 is ineffective, but IL-7/mAb complex can efficiently restore homeostatic defect of aged mice. The fact aging is associated with a defect in the ability to support homeostasis of naïve T cells is shown in FIG. 18A. Here, it is shown that the ability of lymphopenic hosts to support homeostatic expansion declines starting around 1 year of age and becomes severely impaired by 16 months of age. To determine whether the inability of old mice to support homeostatic expansion of naïve T cells can be restored, the effect of injecting free IL-7 and IL-7/mAb complexes was tested. As shown in FIG. 18B, injection of IL-7/mAb complex was able to completely restore the defect in old hosts whereas injecting free IL-7 was ineffective.

FIG. 18 shows that aging is associated with a severe decline in the ability to support homeostatic proliferation of naïve T cells and this can be restored using IL-7 in combination with anti-IL-7 mAb complex. (A) The ability to support homeostasis of naïve T cells declines with age. Groups of B6 mice at various ages (1.5-22 mo) were Irradiated (600 cGy) and injected with 1×10⁶ of CFSE-labeled LN cells from young (2 mo) B6.Thy1.1 mice and the CFSE profiles of donor T cells analyzed 7 d later. Shown are results from host LN; similar data were obtained from host spleen. Each group comprised of 2-3 mice. (B) Effective restoration of homeostatic defect using IL-7-mAb complex. FACS-sorted naïve (CD44^{lo}) B6.Thy-1.1 T cells were CFSE-labeled and injected into irradiated young (2 mo) and old (16 mo) B6 mice. Mice were injected with either rhIL-7 or rhIL-7 plus M25 (anti-hIL-7 mAb) complex every other day and CFSE profile of donor T cells analyzed on d 7 post cell injection. A dose of 1.5 ug rhIL-7 was injected per mouse; the same dose of rhIL-7 was incubated with 15 ug M25 for at least 30 min and injected together as rhIL-7 plus M25 complex. Shown are representative CFSE profiles of three independent experiments with 2-3 mice in each group.

### Example 8

### Converting IL-15 to a Superagonist by Binding to Soluble IL-15Rα

IL-15 is normally presented in vivo as a cell-associated cytokine bound to IL-15Rα. We show here that the biological activity of soluble IL-15 is much improved following interaction with recombinant soluble IL-15Rα; after injection, soluble IL-15/IL-15Rα complexes rapidly induce strong and selective expansion of memory-phenotype CD8⁺ cells and NK cells. These findings imply that binding of IL-15Rα to IL-15 may create a conformational change that potentiates IL-15 recognition by the βγ_{c}, receptor on T cells. The enhancing effect of IL-15Rα binding may explain why IL-15 normally functions as a cell-associated cytokine. Significantly, the results with IL-2, a soluble cytokine, are quite different; thus, IL-2 function is markedly inhibited by binding to soluble IL-2Rα.

In mice, certain cells, namely memory-phenotype (MP) CD8⁺ T cells and NK cells, are highly sensitive to IL-15 (Kennedy et al., J Exp Med 191:771-80, 2000; Judge et al., J Exp. Med 196:935-46, 2002; Fehniger and Caligiuri, Blood 97:14-32, 2001; Becker et al, J Exp Med 195:1541-48, 2002; Zhang et al., Immunity 8:591-99, 1998; Waldmann, T. A., J Clin Immunol 22:51-56, 2002; Zeng et al., J Exp Med 201:139-48, 2005; Van Belle and Grooten, Arch Immunol Ther Exp (Warz) 53:115-26, 2005; Schluns et al., Int J Biochem Cell Biol 37:1567-71, 2005). MP CD8⁺ cells display high levels of CD44 and, like NK cells, also show high expression of CD122 (IL-2Rβ), a component of the receptor for both IL-15 and IL-2 (Waldmann, T. A., J Clin Immunol 22:51-56, 2002). For resting cells, responsiveness to these two cytokines is controlled by a two-chain receptor, βγ_{c}, consisting of the β chain (CD122) plus the common γ chain, γ_{c}, which controls intracellular signalling.

IL-15 is normally not secreted in soluble form (Van Belle and Grooten, Arch Immunol Ther Exp (Warz) 53:115-26, 2005; Schluns et al., Int J Biochem Cell Biol 37:1567-71, 2005; Nguyen et al., J Immunol 169:4279-87, 2002) but is held on the cell surface bound to a unique receptor, IL-15Rα, especially on dendritic cells (DC) (Dubois et al., Immunity 17:597-47, 2002; Burkett et al., J Exp Med 200:825-34, 2004; Burkett et al., Proc Natl Acad Sci USA 100:4724-29, 2003; Schluns et al., Blood 103:988-994, 2004; Zaft et al., J Immunol 175:6428-35, 2005; Sandau et al., J Immunol 173:6537-6541, 2004). Cell-bound IL-15 is then presented in trans to T cells and NK cells and is recognized by the βγ_{c} receptor on these cells; such recognition maintains cell survival and intermittent proliferation.

IL-15Rα plays a mandatory role in presenting endogenous IL-15. Thus, like IL-15^{-/-} mice (1), IL-15Rα^{-/-} mice lack CD122^{hi} CD8⁺ cells and NK cells (Lodolce et al., Immunity 9:669-76, 1998), presumably because the IL-15 synthesized in IL-15R^{-/-} mice fails to leave the cytoplasm. Nevertheless, IL-2Rβγ_{c}⁺ cells can proliferate in response to a soluble recombinant form of IL-15 in the absence of IL-15Rα (Lodolce et al., J Exp Med 194:1187-94, 2001). Moreover, under certain conditions, IL-15Rα can be inhibitory. Thus, injecting mice with a soluble (s) recombinant form of IL-15Rα is reported to suppress NK cell proliferation (Nguyen et al., J Immunol 169:4279-87, 2002) and certain T-dependent immune responses in vivo (Ruckert et al., Eur J Immunol 33:3493-3503, 2003; Ruckert et al., J Immunol 174:5507-15, 2005; Wei et al., J Immunol 167:577-82, 2001; Ruchatz et al., J Immunol 160:5654-5660, 1998), and adding sIL-15Rα in vitro can block the response of cell lines to IL-15 (Ruckert et al., J Immunol 174:5507-15, 2005; Wei et al., J Immunol 167:577-82, 2001; Ruchatz et al., J Immunol 160:5654-5660, 1998; Budagian et al., J Biol Chem 279:40368-75, 2004; Mortier et al., J Immunol 173:1681-1688, 2004; Eisenman et al., Cytokine 20:121-29, 2002). Despite these findings, there are other reports that sIL-15Rα (Giron-Michel et al., Blood 106:2302-10, 2005), and also a soluble sushi domain of IL-15Rα (Mortier et al., J Biol Chem, 2005, E-pub ahead of print), can enhance IL-15 responses of human cell lines.

### Example 9

### Stimulation by IL-15/IL-15Rα Complexes In Vitro

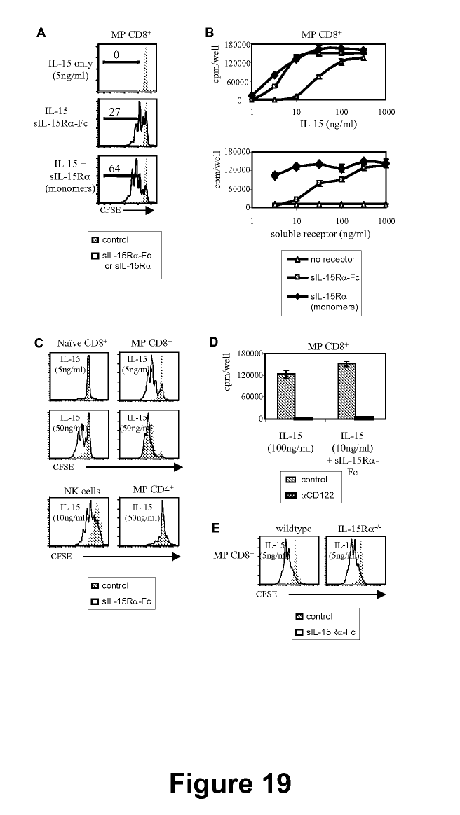

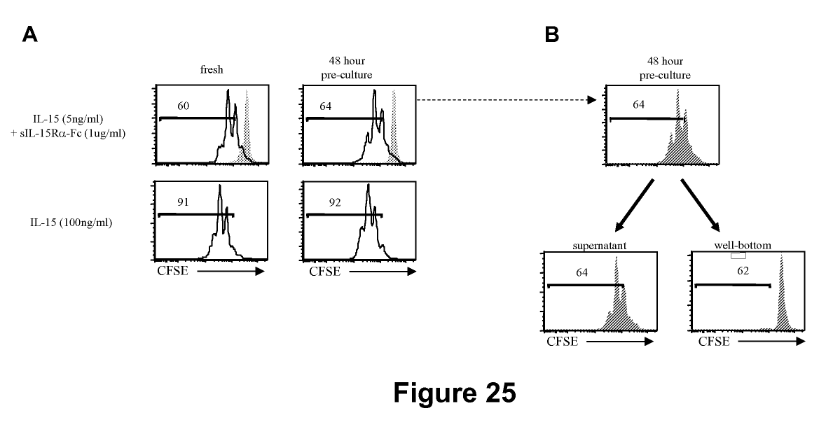

To examine whether the stimulatory function of soluble IL-15 is altered by binding to sIL-15Rα, purified MP CD44^{hi} CD122^{hi} CD8⁺ cells were cultured in vitro with mouse IL-15±mouse sIL-15Rα covalently linked to the Fc portion of human IgG1 (sIL-15Rα-Fc). For IL-15 alone, half-maximal responses required about 30 ng/ml and responses were negligible with <10 ng/ml (FIG. 19A, 19B). Here, the notable finding was that supplementing a low concentration of IL-15, e.g. 5 ng/ml, with sIL-15Rα-Fc led to strong proliferative responses of MP CD8⁺ cells as measured either by CFSE dilution (FIG. 19A) or [³H]-thymidine incorporation (FIG. 19B). No proliferation occurred with sIL-15Rα-Fc alone (FIG. 19B), and addition of sIL-15Rα-Fc failed to alter the response of MP CD8⁺ cells to a different cytokine, IL-2 (data not shown). With IL-15, we could find no evidence that sIL-15Rα-Fc acted by enhancing the half-life of IL-15 in vitro (FIG. 25).

With a limiting concentration of cytokine, IL-15 responses were generally improved by 6-9 fold by addition of sIL-15Rα-Fc. Adding sIL-15Rα-Fc also considerably improved the IL-15 response of CD122^{hi} NK cells (FIG. 19C), but was relatively ineffective on MP (CD44^{hi}) CD4⁺ cells which express intermediate levels of CD122 (FIG. 19C). Unexpectedly, sIL-15Rα-Fc plus IL-15 led to significant proliferation of typical naïve CD44^{lo} CD122^{lo} CD8⁺ cells, though only with high concentrations of IL-15 (FIG. 19C).

For MP CD8⁺ cells, responses to both soluble IL-15 alone and IL-15 plus sIL-15Rα-Fc were mediated solely through βγ_{c} receptors. Thus, responses were abolished by addition of CD122 mAb (FIG. 19D) and were as high with MP CD8⁺ cells from IL-15Rα^{-/-} mice as with normal MP CD8⁺ cells (FIG. 19E).

Being a dimeric molecule, sIL-15Rα-Fc might enhance IL-15 activity by presenting this cytokine in cross-linked form. However, enzyme-cleaved monomeric fragments of sIL-15Rα-Fc were no less potent in augmenting IL-15 responses than dimeric molecules (FIG. 19A, 19B). Indeed, under limiting conditions, responses were appreciably higher with the receptor monomers than with the Fc dimers (FIG. 19B). Why the receptor monomers were more effective than the dimers is unclear, although for steric reasons the monomer/IL-15 complexes may bind more effectively to the βγ_{c} receptor.

FIGS. 19A, 19B, 19C, 19D, and 19E show soluble IL-15Rα augments IL-15-mediated lymphocyte proliferation in vitro. (A) Purified MP (CD44^{hi}) CD8⁺ T cells were labeled with CFSE and cultured at 5×10⁴ cells/well with 5 ng/ml of IL-15. As indicated, 1 μg/ml of either sIL-15Rα-Fc (dimers) or sIL-15Rα (monomers) was added to the cultures. CFSE dilution was assessed on day 4. Representative data are shown. (B) Purified MP CD8⁺ T cells were cultured with either titrated amounts of IL-15 plus a fixed concentration of soluble receptor (1 μg/ml) (top) or titrated amounts of soluble receptor plus a fixed concentration of IL-15 (10 ng/ml) (bottom). The data show mean levels of [³H]-thymidine incorporation (±SD) for triplicate cultures on day 3. (C) Purified naïve (CD44^{lo}) CD8⁺ T cells, MP CD8⁺ T cells, NK cells, or MP CD4⁺T cells were cultured with IL-15 as indicated. Soluble IL-15Rα-Fc was added at 1 μg/ml. CFSE dilution was assessed on day 3. (D) As in (B) except 10 μg/ml of anti-CD122 antibody was added as indicated. (E) MP CD8⁺ T cells from wild type Ly5.2 and IL-15Rα^{-/-} /Ly5.1 mice were mixed together, labeled with CFSE, and cultured as indicated. CFSE dilution on Ly5.1⁻ (wild type) and Ly5.1 (IL-15Rα^{-/-}) cells was measured on day 3.

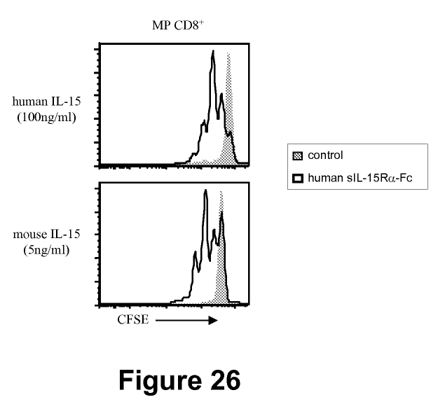

The above data refer to mouse IL-15 and mouse soluble IL-15Rα. Quite similar data applied to human IL-15/IL-15Rα. Thus, the response of mouse MP CD8⁺ cells to either human or mouse IL-15 was considerably enhanced by addition of human sIL-15Rα-Fc (FIG. 26). Addition of human IL-15Rα monomers was even more effective. Note that, for mouse IL-2Rβγ_{c} responses, human IL-15 is considerably weaker than mouse IL-15 (Eisenman et al., Cytokine 20:121-29, 2002).

FIGS. 25A and 25B show survival of IL-15 in vitro. (A) Purified CFSE-labeled MP CD8⁺ T cells were added to cultures containing IL-15 alone at 5 ng/ml (top, grey), 100 ng/ml (bottom), or 5 ng/ml of IL-15 plus 1 μg/ml sIL-15Rα-Fc (top, solid line). These cultures were either freshly prepared (fresh) or were left for 48 hours at 37° C. (48 hours pre-culture) before addition of T cells. (B), as for (A) except that T cells were cultured with supernatants taken from the “48 hour pre-cultures” (supernatant) vs the latter cultures that had been emptied of supernatant without washing (well-bottom). Interpretation: The experiment shows that the biological activity of IL-15 (cultured alone) did not decline significantly during culture for 48 hours at 37° C., thus making it unlikely that sIL-15Rα-Fc acted simply by prolonging the half-life of IL-15. Furthermore, the absence of proliferation of cells transferred to the emptied wells (well-bottom) suggests that the enhancing activity of the soluble receptor did not reflect cross-linked presentation of IL-15 bound via the receptors to the plastic bottom of the well.

FIG. 26 shows human sIL-15Rα-Fc enhances the response of mouse MP CD8⁺ cells to both mouse and human IL-15. CFSE-labeled purified MP CD8⁺ T cells were cultured at 5×10⁴ cells/well with either 100 ng/ml of human IL-15 or 5 ng/ml of murine IL-15. As indicated, 1 μg/ml of human sIL-15Rα-Fc was added to the cultures. CFSE dilution was assessed after 3 days of culture. Note that, in direct contrast to CTLL (which express IL-15Rα plus βγ_{c} mouse MP CD8⁺ cells respond better to mouse IL-15 than to human IL-15. Eisenman, J., et al., Cytokine 20: 121-129, 2002.

### Example 10

### In Vivo Responses

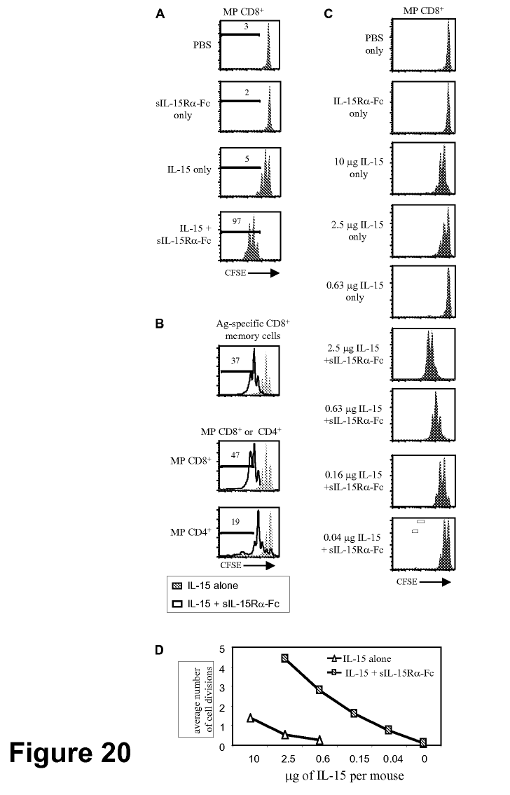

Confirming previous findings (Judge et al., J Exp. Med 196:935-46, 2002; Zhang et al., Immunity 8:591-99, 1998), injecting mice ip with IL-15 after iv injection of CFSE-labeled MP CD8⁺ cells caused about 50% of the donor cells to divide 1-2 times (FIG. 20A). With coinjection of sIL-15Rα-Fc, however, virtually all of the donor cells divided and >95% of the cells divided 3 times or more (compared with <5% for IL-15 injected alone); by contrast, injection of sIL-15Rα-Fc alone had no effect on proliferation. The capacity of sIL-15Rα-Fc to enhance responses of MP CD8⁺ cells to IL-15 also applied to antigen-specific memory CD8⁺ cells, i.e. to antigen-primed P14 TCR transgenic CD8⁺ cells (FIG. 20B, top). There was also enhancement of the IL-15 response of MP CD4⁺ cells (FIG. 20B, bottom).

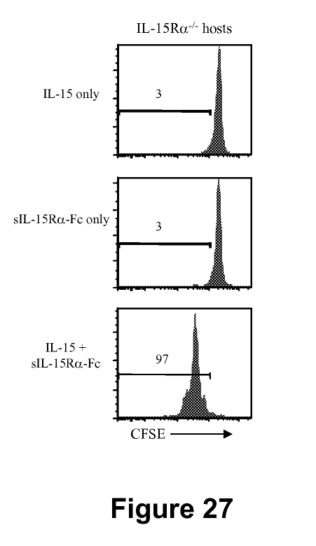

For MP CD8⁺ cells, IL-15 titration experiments showed that in vivo responses to IL-15 were increased about 50-fold when limiting doses of IL-15 were coinjected with sIL-15Rα-Fc (FIG. 20C, 20D). Endogenous IL-15Rα was not required because similar data applied with T cell transfer to IL-15Rα^{-/-} hosts (FIG. 27).

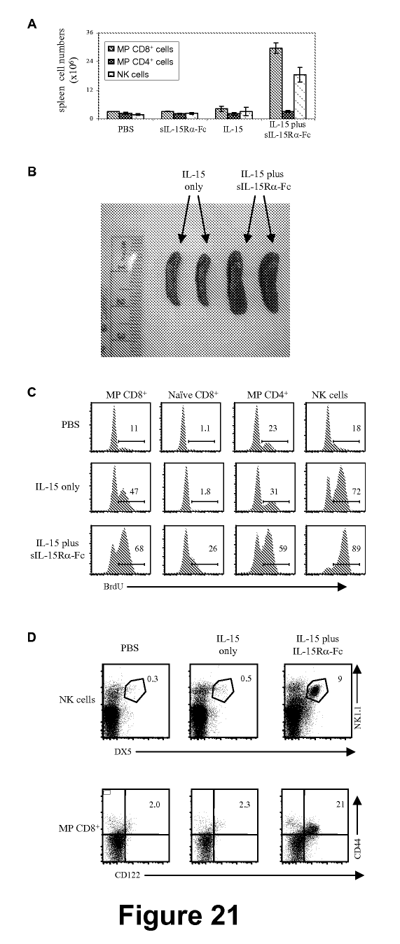

The above data apply to CFSE-labeled donor cells. For host cells, injection of IL-15 or sIL-15Rα-Fc alone had little effect on cell numbers. By contrast, two injections of IL-15 plus sIL-15Rα-Fc caused a marked increase in total numbers of host MP CD8⁺ cells and NK cells by day 3 after initial injection and the spleen was obviously enlarged (FIG. 21A, 21B). Likewise, proliferation as measured by BrdU incorporation was much higher with injection of IL-15 plus sIL-15Rα-Fc than with IL-15 alone (FIG. 21C).

IL-15Rα^{-/-} mice lack CD122^{hi} MP CD8⁺ cells and NK cells (Lodolce et al., Immunity 9:669-76, 1998), presumably because the absence of IL-15Rα precludes presentation of endogenous IL-15. As shown in FIG. 21D, injecting IL-15Rα^{-/-} mice with a mixture of IL-15 and sIL-15Rα-Fc rapidly restored numbers of host NK1.1⁺DX5⁺NK cells and CD122^{hi} MP CD8⁺ cells; at the dose used, IL-15 alone was ineffective.

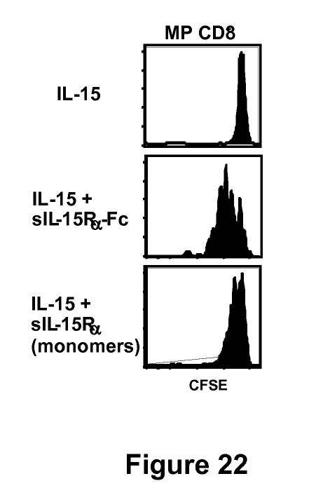

The above in vivo effect applied to IL-15 complexed with dimeric IL-15Rα-Fc. To determine whether Fc component is required, the ability of IL-15 complexes generated with monomeric IL-15Rα devoid of Fc to induce proliferation of MP CD8⁺ cells was tested under in vivo conditions. Strikingly, while monomer IL-15/sIL-15Rα complexes induced higher proliferation of MP CD8⁺ cells under in vitro conditions than dimeric IL-15/IL-15Rα-Fc complexes (FIG. 19A), the opposite was the case under in vivo conditions (FIG. 22). Thus, in contrast to the potent activity of dimeric IL-15/sIL-15Rα-Fc complexes, monomer IL-15/sIL-15Rα complexes depleted of the Fc portion displayed only slightly better in vivo activity than free IL-15 (FIG. 22). The Fc part of the receptor therefore appears important for the in vivo activity of the complexes.

FIGS. 20A, 20B, 20C, and 20D show soluble IL-15Rα augments IL-15-mediated donor lymphocyte proliferation in vivo. (A) CFSE-labeled T cells were transferred iv into C57BL/6 (B6) recipients. On days 1 and 2 after transfer, the recipients were given ip injections of PBS, sIL-15Rα-Fc alone (7 μg), IL-15 alone (1.5 μg), or sIL-15Rα-Fc plus IL-15 (7 μg and 1.5 μg, respectively, which represents a 1:2 molar ratio). CFSE dilution of the donor cells was measured in spleen on day 4. Representative data for gated MP CD8⁺ cells are shown. (B) As in (A) except that the cells transferred were from LCMV-immune mice (top) versus normal mice (bottom). (C) CFSE-labeled MP CD8⁺ T cells were transferred to normal B6 hosts; one day later, the hosts were injected with the indicated dose of IL-15 with or without sIL-15Rα-Fc; the dose of sIL-15Rα-Fc varied such that a 2:1 molar ratio of IL-15 to sIL-15Rα-Fc was injected. CFSE profiles for donor MP CD8⁺ cells in spleen at 2 days after injection are shown. (D) Compilation of data from (C). For A, B, and C, data shown are representative of 2 mice per group and are also representative of 2 independent experiments.

FIGS. 21A, 21B, 21C, and 21D show soluble IL-15Rα augments IL-15-mediated host lymphocyte proliferation. (A) Normal B6 mice were injected iv on days 1 and 2 with PBS, sIL-15Rα-Fc alone, IL-15 alone, or sIL-15Rα-Fc/IL-15 as described for FIG. 20A. Total numbers of CD8⁺ MP T cells, CD4⁺ MP T cells, and NK cells recovered from spleen on day 3 are shown. (B) Spleens from (A) were photographed as indicated. (C) Mice were treated as in (A), except that the mice were also given an iv injection of BrdU on day 1 and placed on BrdU in the drinking water until sacrifice. Shown is BrdU staining for MP CD8⁺, naïve CD8⁺, MP CD4⁺, and NK cells. (D) IL-15Rα^{-/-} mice were injected iv on days 1, 3, 5, and 7, with either PBS, IL-15 (0.6 μg), or IL-15 (0.6 μg)/sIL-15Rα-Fc (1 μg). The data show staining of spleen cells on day 9. For B, C, D, representative data are shown. All data are representative of at least 2 independent experiments.

FIG. 22 shows sIL-15Rα-Fc (dimers) are better than sIL-15Rα (monomers) under in vivo conditions. CFSE-labeled Thy-1.1 MP CD8 cells were injected into normal B6 hosts and injected with 1 μg IL-15, 1 μg IL-15+5 μg sIL-15Rα-Fc, or 1 μg sIL-15Rα-Fc+10 sIL-15Rα and then analyzed on d3. Shown are CFSE profiles on donor CD8 cells recovered from host spleen.

FIG. 27 shows stimulation by IL-15/sIL-15-Rα-Fc complexes in IL-15Rα^{-/-} hosts. Purified CFSE-labeled MP CD8⁺T cells were transferred iv into IL-15Rα^{-/-} recipients. On day 1 after transfer, recipient mice were given ip injections of PBS, sIL-15Rα-Fc alone (10 μg), IL-15 alone (2 μg), or sIL-15Rα-Fc plus IL-15 (10 μg and 2 μg, respectively). On day 3 after transfer, spleen cells were harvested and CFSE dilution was assessed by flow cytometric analysis.

### Example 11

### Failure of sIL-15Rα-Fc to Block Presentation of Endogenous IL-15

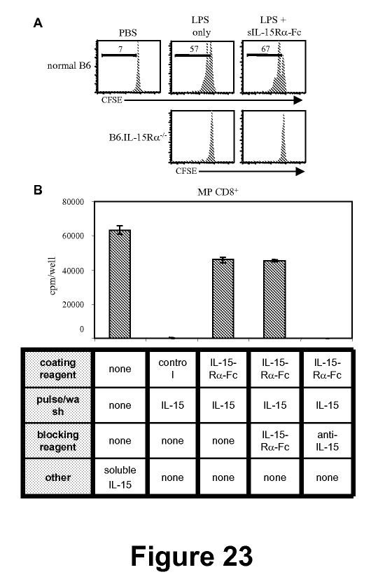

Injecting mice with LPS is known to cause a brief increase in endogenous IL-15 (and IL-15Rα) synthesis by non-T cells in vivo, with a consequent transient increase in the proliferation rate of IL-15-responsive CD122^{hi} MP CD8⁺ cells. Mattei et al., J Immunol 167:1179, 2001; Tough et al., J Exp Med 185:2089, 1997. Such LPS-induced bystander proliferation is illustrated in FIG. 23A where most donor MP CD8⁺ cells underwent 1-2 cell divisions by day 3 following exposure to LPS in normal B6 hosts, which contrasted with the lack of proliferation in IL-15Rα^{-/-} hosts. Significantly, injecting sIL-15Rα-Fc after LPS injection failed to reduce proliferation, even with daily injections of sIL-15Rα-Fc (10 μg/injection). Hence, injection of sIL-15Rα-Fc was unable to block T cell contact with endogenous IL-15 bound to endogenous IL-15Rα. Also, for IL-15Rα^{-/-} hosts, sIL-15Rα-Fc was clearly unable to compensate for the lack of endogenous IL-15Rα, presumably because the latter is essential for conveying IL-15 to the cell surface.

Similar findings applied to an in vitro system where MP CD8⁺ cells were cultured in wells that were first coated with sIL-15Rα-Fc and then pulsed with IL-15, followed by thorough washing to remove unbound cytokine (FIG. 23B). Thus, proliferative responses elicited by the bound IL-15Rα-Fc/IL-15 complexes could not be inhibited by addition of soluble (unbound) IL-15Rα-Fc as a blocking reagent. By contrast, addition of a polyclonal antibody against IL-15 abolished proliferation.

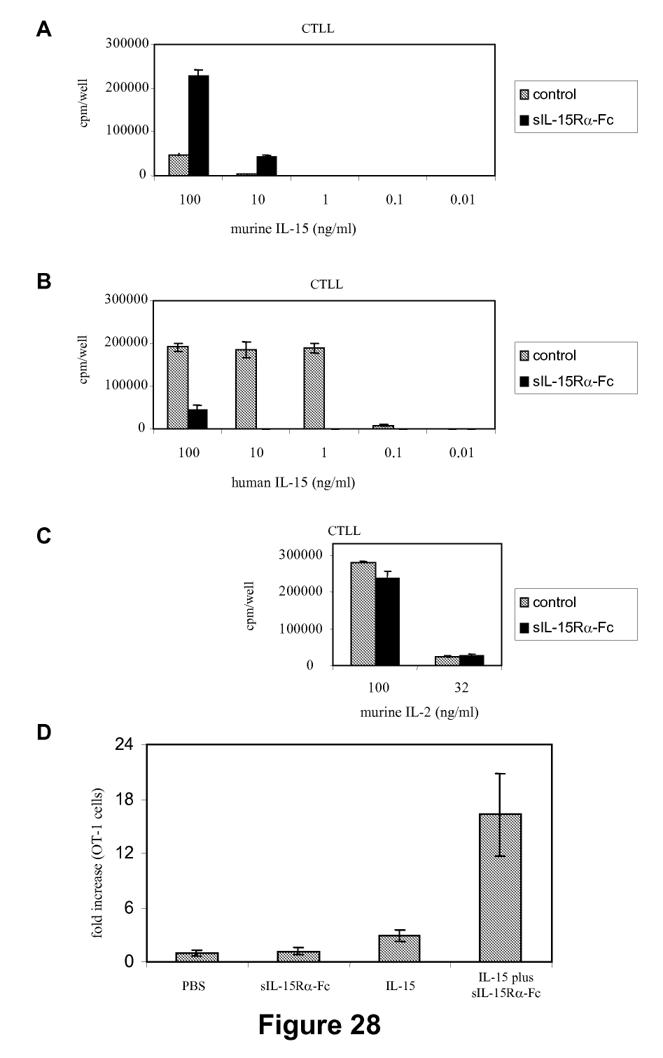

Based on the above findings, the IL-15 molecule has only a single binding site for interaction with IL-15Rα. Once this site is occluded, either by binding to endogenous IL-15Rα on cells in vivo or to IL-15Rα attached to plastic in vitro, interaction with exogenous sIL-15Rα-Fc fails to occur and there is no interference with presentation of IL-15 to T cells. This scenario does not explain why sIL-15Rα can block the response of cell lines to IL-15 ((Ruckert et al., J Immunol 174:5507-15, 2005; Wei et al., J Immunol 167:577-82, 2001; Ruchatz et al., J Immunol 160:5654-5660, 1998; Budagian et al., J Biol Chem 279:40368-75, 2004; Mortier et al., J Immunol 173:1681-1688, 2004; Eisenman et al., Cytokine 20:121-29, 2002). Here it may be relevant that these studies used human or simian IL-15, and not mouse IL-15 as in our study, which raises the possibility of distinct species differences in IL-15. In favor of this idea, we found that, as for MP CD8⁺ cells, the response of mouse CTLL cells to mouse IL-15 was enhanced by mouse sIL-15Rα-Fc (FIG. 28A). By contrast, confirming the findings of others (Ruckert et al., J Immunol 174:5507-15, 2005; Wei et al., J Immunol 167:577-82, 2001; Ruchatz et al., J Immunol 160:5654-5660, 1998; Budagian et al., J Biol Chem 279:40368-75, 2004), the high response of CTLL cells to human IL-15 (Eisenman et al., Cytokine 20:121-29, 2002) was strongly inhibited by mouse sIL-15Rα-Fc (FIG. 28B). CTLL responses to IL-2 as a control were not affected by adding sIL-15Rα-Fc (FIG. 28C).

The above findings do not explain the reports that murine sIL-15Rα constructs are inhibitory for NK cell proliferation (Nguyen et al., J Immunol 169:4279-87, 2002) and antigen-driven T cell responses in vivo (Ruckert et al., Eur J Immunol 33:3493-3503, 2003; Ruckert et al., J Immunol 174:5507-15, 2005; Wei et al., J Immunol 167:577-82, 2001; Ruchatz et al., J Immunol 160:5654-5660, 1998). This discrepancy has yet to be resolved, although it is of interest that antigen-specific proliferative responses of naïve OT-1 TCR transgenic CD8⁺ cells to specific peptide in vivo were not blocked by coinjection of sIL-15Rα-Fc, and the responses were considerably enhanced when a mixture of sIL-15Rα and IL-15 was injected (FIG. 28D). As yet we have not used the very large doses of sIL-15Rα required to block in vivo responses, i.e. 400 μg/injection for NK cell proliferation (Nguyen et al., J Immunol 169:4279-87, 2002). Also, it is possibly relevant that the studies showing inhibition by sIL-15Rα in vivo used constructs grown in bacteria, whereas our constructs were grown in mammalian cells.

FIGS. 23A and 23B show proliferation to IL-15 immobilized by IL-15Rα cannot be blocked by soluble IL-15Rα-Fc. (A) CFSE-labeled T cells were injected iv into Thy1-congenic B6 or IL-15Rα^{-/-} hosts. One day later, mice were injected ip with PBS or 500 ng of LPS. As indicated, mice were also treated ip with 10 μg of sIL-15Rα-Fc daily beginning the day of LPS injection. Three days after LPS injection, mice were sacrificed, and CFSE dilution of MP CD8⁺ T cells was assessed. (B) 96-well plates were pre-coated overnight with 10 μg/ml of sIL-15Rα-Fc. Plates were then washed and incubated with 1 μg/ml IL-15 for 1 hour at 37 degrees. Thereafter, plates were washed and 5×10⁴ MP CD8⁺ T cells were added together with 1) 10 μg/ml sIL-15Rα-Fc, 2) 10 μg/ml of polyclonal anti-IL-15 antibody, or 3) control media; as an additional control, free IL-15 (32 ng/ml) was added to some wells. The data show mean levels of [³H]-thymidine incorporation (SD) for triplicate cultures on day 3.

FIG. 28A, 28B, and 28C show blocking effects of sIL-15Rα-Fc for responses to mouse vs human IL-15. (A, B, C) CTLL-2 cells were cultured for 2 days with either (A) murine IL-15, (B) human IL-15, or (C) murine IL-2. As indicated, cultures were supplemented with 1 μg/ml of murine sIL-15Rα-Fc. [³H]-thymidine as added during the last 24 hours of culture. The data show mean levels of [³H]-thymidine incorporation (±SD) for triplicate cultures. (D) One million OT-1 cells (Thy1.1 congenic) were adoptively transferred iv into B6 recipient mice on day −1. On day 0, mice were vaccinated with one million SIINFEKL-pulsed dendritic cells iv. On days 1-7, recipient mice were given daily ip injections of PBS, sIL-15Rα-Fc alone (5 μg), IL-15 alone (1 μg), or sIL-15Rα-Fc plus IL-15 (5 μg and 1 μg, respectively). On day 8, spleens were harvested, counted, and evaluated by flow cytometric analysis for donor OT-1 cells. The data show fold increase of absolute numbers of OT-1 cells relative to vaccination without cytokine or receptor treatment. All data are representative of at least 2 independent experiments.

### Example 12

### Stimulation by IL-2 Plus IL-2Rα

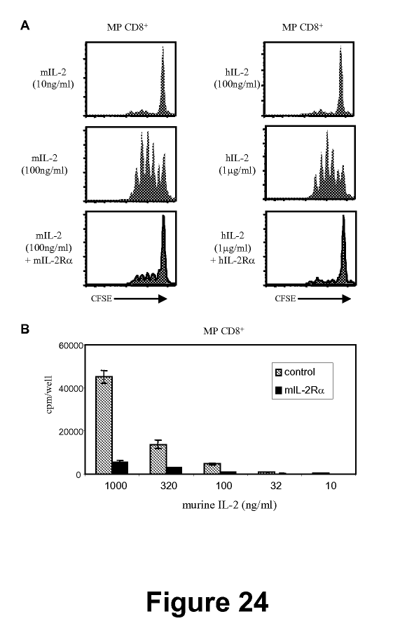

The observation that the biological activity of IL-15 was enhanced by binding to soluble IL-15Rα raised the question whether comparable findings would apply to IL-2 and IL-2Rα (CD25). As shown in FIG. 24, this was clearly not the case. Thus, proliferative responses of MP CD8⁺ cells to mouse IL-2 in vitro were markedly inhibited by addition of soluble mouse IL-2Rα (FIG. 24A left, 24B). Similar inhibition applied to MP CD8⁺ cells (mouse) responding to human IL-2 and soluble human IL-2Rα. (FIG. 24A right).

Thus, whereas soluble IL-15Rα potentiated the function of IL-15, soluble IL-2Rα blocked the function of IL-2.

FIGS. 24A and 24B show soluble IL-2Rα inhibits IL-2-mediated proliferation. (A) Purified CFSE-labeled MP CD8⁺T cells were cultured with either murine IL-2 or human IL-2 at the concentration shown. As indicated, 2.5 ng/ml of either soluble murine IL-2Rα or soluble human IL-2Rα was added to the cultures. CFSE dilution was assessed on day 3. Representative data are shown. (B) Purified MP CD8⁺ T cells were cultured with titrated amounts of murine IL-2 with or without soluble mIL-2Rα (2.5 μg/ml). The data show mean levels of [³H]-thymidine incorporation (±SD) for triplicate cultures on day 3.

### Example 13

### Soluble Complexes of IL-15 and IL-15Rα are More Stimulatory than Soluble IL-15 Alone

The main conclusion from the above experiments is that soluble complexes of IL-15 and IL-15Rα are much more stimulatory than soluble IL-15 alone, both in vivo and in vitro. Without structural studies on IL-15/IL-15Rα interaction, one can only speculate on why and how this interaction potentiates IL-15 function. There are several possibilities:

First, binding of IL-15Rα to IL-15 might impair IL-15 internalization by T cells and thereby strengthen signaling through the βγ_{c} receptor. This idea is in line with reports that internalization of certain cytokines, e.g. IL-2, serves to attenuate receptor signaling (Chang et al., J Biol Chem 271:13349-55, 1996). However, we do not favor this possibility for two reasons. First, if the strong stimulation by IL-15/sIL-15Rα complexes reflected reduced IL-15 internalization, one would expect to see a parallel reduction in internalization of CD122, the receptor for IL-15. As measured by downregulation from the cell surface, however, the opposite applies, i.e. greater downregulation of CD122 with IL-15/sIL-15Rα complexes than with IL-15 alone (data not shown). The second argument against IL-15/sIL-15Rα complexes preventing IL-15 internalization is that, if this were the case, we should have seen similar findings with IL-2/sIL-2Rα, which was not so. Thus, IL-2/sIL-2Rα complexes were much less stimulatory than soluble IL-2 alone, which clearly contrasted with IL-15Rα/IL-15 complexes being more stimulatory than IL-15 alone.

A second possibility for how sIL-15Rα potentiates IL-15 activity is that sIL-15Rα might prevent degradation of IL-15. This notion deserves consideration because the enhancing effect of sIL-15Rα-Fc on IL-15 function was more pronounced in vivo than in vitro. Here, it is notable that binding of certain cytokines to antibodies or soluble receptors can extend cytokine survival in vivo (Finkelman et al., J Immunol 151:1235-44, 1993; Ma et al., J Pharmacol Exp Ther 279:340-50, 1996; Peters et al., J Exp Med 183:1399-1406, 1996; Rosenblum et al., Cancer Res 45:2421-24, 1985; Peleg-Shulman et al., J Biol Chem 279:18046-18053, 2004; Kobayashi et al., Cytokine 11:1065-75, 1999). Hence, sIL-15Rα-Fc binding to IL-15 may increase the half-life of IL-15 in vivo. Notably, however, we failed to observe an increase in IL-15 half-life in vitro.

In light of the above, we favor a third possibility, namely that IL-15Rα improves the function of IL-15 by inducing a conformational change in IL-15, which augments interaction with the βγ_{c} receptor, thus changing IL-15 from an agonist to a superagonist. This model is in line with the affinity of IL-15/IL-15Rα interaction being far higher than for IL-2/IL-2Rα interaction (Fehniger and Caligiuri, Blood 97:14-32, 2001; Van Belle and Grooten, Arch Immunol Ther Exp (Warz) 53:115-26, 2005) and explains why, unlike IL-2, IL-15 functions so well as a cell-associated cytokine. Testing this idea directly will obviously require structural studies. In this respect, it is notable that the interaction between IL-15 and IL-15Rα involves a unique network of ionic interactions not found in other cytokine/cytokine receptor complexes (Lorenzen et al., J Biol Chem, 2005, E-pub ahead of print). Whether this unique interaction results in a conformational change in IL-15 has yet to be determined.

There is accumulating evidence that IL-15 has beneficial effects on T cell survival and memory generation and also has potential for restoring the T cell pool after irradiation and other forms of cytoreduction (Becker et al, J Exp Med 195:1541-48, 2002; Zhang et al., Immunity 8:591-99, 1998; Waldmann, T. A., J Clin Immunol 22:51-56, 2002; Zeng et al., J Exp Med 201:139-48, 2005; Van Belle and Grooten, Arch Immunol Ther Exp (Warz) 53:115-26, 2005; Schluns et al., Int J Biochem Cell Biol 37:1567-71, 2005; Lodolce et al., J Exp Med 194:1187-94, 2001; Rubinstein et al., J Immunol 169:4928-35, 2002; Diab et al, Cytotherapy 7:23-35, 2005). As shown here, the biological activity of IL-15 as a therapeutic reagent could be considerably enhanced by administering preformed soluble IL-15/IL-15R complexes.

### Example 14

### Materials and Methods

Mice. C57BL/6 (B6), B6.Ly5.1, B6.Thy1.1, and OT-1 mice were purchased from Jackson Laboratory (Bar Harbor, Me.). IL-15Rα^{-/-} mice (Lodolce et al., Immunity 9:669-76, 1998) were a generous gift of Averil Ma (University of California, San Francisco) and IL-7 transgenic (tg) mice (Mertshing et al., Int Immunol 7:401-14, 1995) were a generous gift of J. Andersson (Basel Institute, Switzerland). P14 TCR tg mice were kindly provided by J. Lindsay Whitton (Scripps Research Institute). IL-15Rα^{-/-}, IL-7 μg, P14, and OT-1 TCR tg mice were all maintained on a B6 background and for some experiments crossed to either B6.Ly5.1 or B6.Thy1.1 mice. IL-15Rα^{-/-} mice were crossed to IL-7 tg mice to generate IL-7 tg/IL-15Rα^{-/-} mice. As we have previously described with IL-7 tg/IL-15^{-/-} mice (Kieper et al., J Exp Med 195:1533-39, 2002), IL-7 tg/IL-15Rα^{-/-} mice have similar large numbers of CD122^{hi} MP CD8⁺ T cells as IL-7 tg mice.

Recombinant Proteins. Murine sIL-15Rα-Fc, human sIL-15Rα-Fc, and human IL-2Rα were purchased from R&D systems (Minneapolis, Minn.). Monomeric sIL-15Rα and mouse IL-2Rα were purchased from R&D systems as prerelease reagents. Monomeric sIL-15Rα was generated by enzyme digestion of the dimeric sIL-15Rα. We verified complete digestion by western blot using anti-IL-15Rα polyclonal antibodies (AF551 and BAF551, R&D systems) (data not shown). Recombinant cytokines (including mouse IL-15, human IL-15, mouse IL-2, human IL-2, mouse IL-4, and mouse GM-CSF) were purchased from Ebioscience and/or R&D systems.

Isolation of T cells and CFSE labeling. To obtain adequate numbers of cells, in most experiments MP CD8⁺ cells were prepared from IL-7 transgenic mice. By all parameters tested MP CD8⁺ cells from IL-7 tg mice are identical to cells from normal mice. Moreover, the main findings reported here for IL-15/sIL-15Rα-Fc complexes were also observed with cells prepared from normal mice, both in vivo and in vitro. MP CD8⁺T cells used for either in vitro or adoptive transfer experiments were isolated from LN and spleen, and purified by cell sorting. In brief, single cell suspensions were first enriched for CD3 T cells using a mouse T cell enrichment columns (MTCC-25, R&D systems, Minneapolis, Minn.). Enriched T cells were labeled with antibodies and purified by cell sorting for CD8⁺ CD44^{hi} T cells. In some experiments, we used a similar protocol and isolated CD8⁺CD44^{lo}, CD4⁺ CD44^{hi}, NK1.1⁺/DX5⁺ cells. Cell sorting was performed using a BD FACSAria. Purity of sorted cells was routinely tested and over 98%. In some experiments, total T cells or OT-1 cells were used as donor lymphocytes. For these experiments, cells from spleen and LN were purified using a mouse T cell enrichment column (MTCC-25). For experiments using CFSE-labeled cells, T cells were labeled with 1.5 μm CFSE (Molecular Probes, Eugene, Oreg.) according to the manufacturer's directions.

Generation of antigen-specific CD8⁺T cells. We generated antigen-specific memory T cells following adoptive transfer of P14 TCR tg CD8⁺T cells (which recognize the LCMV gp33 peptide) and LCMV infection. Briefly, 5×10⁴ P14 TCR transgenic CD8⁺T cells were adoptively transferred into Thy1 congenic IL-7 tg recipient mice. Twenty-four hours later, mice received 2×10⁵ plaque-forming units of the LCMV Armstrong strain. Two months after viral infection, T cells were isolated using a mouse T cell enrichment column (MTCC-25), labeled with CFSE, and adoptively transferred into Ly5 congenic recipient mice. Donor P14 CD8⁺T cells (Thy1.1) represented 15-20% of the donor CD8⁺T cell (Ly5.2) population.

In vitro assays. All cultures were performed in RPMI 1640 supplemented with 10% FCS, glutamine, 2-ME, non-essential amino acids, and antibiotics. FACS-purified T cells and NK cells were isolated as described above. CTLL (CTLL-2) cells were obtained from ATCC (Manassas, Va.), and cultured in RPMI medium supplemented with murine IL-2. For experiments with FACS-purified lymphocytes, 5×10⁴ cells in 200 ul were plated per well in 96 well plates. Cytokine and/or soluble receptor were added at concentrations described in the figure or figure legend. For CD122 blocking experiments, we used purified anti-CD122 antibody (TM-β1 (NA/LE), BD Pharmingen). For experiments to block plate-bound IL-15, polyclonal anti-IL-15 antibody (AF447, R&D systems) was used. Experiments with CTLL cells were plated as with FACS-purified lymphocytes except using 2×10⁴ cells per well. For proliferation experiments with [³H]-thymidine, 1 μCi/ml was added as indicated in the figure legend. Cells were cultured in triplicate wells.

In vivo assays. For experiments assessing proliferation of adoptively transferred cells, T cells were isolated and labeled with CFSE (as described above), and then injected iv into Ly5 or Thy1 congenic recipient mice. In experiments to measure proliferation of host cells, mice were injected ip with BrdU (2 mg) and then maintained on BrdU drinking water (0.8 mg/ml) using methodology previously described (Judge et al., J Exp. Med 196:935-46, 2002). For injections of cytokine and soluble receptor, IL-15 and sIL-15Rα-FC were incubated together for 20 minutes at 37° C. Samples were then diluted at least 10 fold in PBS to a volume of 500 ul prior to injection into mice. In control conditions, cytokine or receptor alone was also incubated for 20 minutes at 37° C. LPS (ALX-581-008, Alexis Biochemicals, San Diego, Calif.) were injected ip in PBS. For vaccination experiments, dendritic cells were prepared as previously described by culture of bone marrow cells with GM-CSF and IL-4 (Rubinstein et al., J Immunol 169:4928-35, 2002). Dendritic cells were pulsed for 2 hours with SIINFEKL peptide at 37° C., washed, and injected iv.

Flow Cytometric Analysis. Cells were analyzed by flow cytometric analysis using standard protocols. Briefly, cells were washed in FACS buffer containing 1% FCS and 2 mM EDTA, and stained with combinations of the antibodies: CD8-PerCP-Cy5.5, -APC, or -APC-Cy7 (53-6.7, eBioscience and BD Pharmingen), CD49b-PE and -APC (DX5, eBioscience), NK1.1-FITC and -PE (PK136, BD Pharmingen), CD3-PE, -PerCP-Cy5.5, -PE-Cy7, or -APC (145-2C11, eBioscience and BD Pharmingen), CD3-Pacific Blue (500A2, BD Pharmingen), CD4-PE, PE-Cy7, or -APC (RM4-5, eBioscience and BD Pharmingen), Ly5.1-FITC, —PE, -PE-Cy7, and -APC (A20, eBioscience and BD Pharmingen), Ly5.2-FITC, -PE, -PerCP-Cy5.5, and -APC (104, eBioscience and BD Pharmingen), Thy1.1-FITC, PE, -PE-Cy7, and -APC (HIS51, eBioscience), Thy1.2-FITC, PE, and -APC (53-2.1, eBioscience), CD44-FITC -APC, and -Alexa Fluor 405 (IM7, eBioscience and Caltag Laboratories (Burlingame, Calif.)), CD122-PE (TM-131, BD Pharmingen), B220-PerCP-Cy5.5 (RA3-6B2, BD Pharmingen), and TCR Vα2-PE (B20.1, BD Pharmingen). BrdU intracellular staining was performed with reagents from FITC or APC BrdU flow kits (559619 and 552598, BD Pharmingen) according to the manufacturer's directions. Flow cytometric samples were analyzed using a BD LSR II digital flow cytometer (BD Biosciences, San Jose, Calif.). Data was analyzed using FlowJo software (Tree Star, San Carlos, Calif.).

### Example 15

### Materials and Methods

Mice. C57BL/6 (B6), B6.PL (Thy1.1-congenic), IL-2^{+/-} and CD25^{+/-} mice, all on a B6 background, were purchased from The Jackson Laboratory (Bar Harbor, Me.). IL-7 transgenic (tg) mice and P14 tg mice, both on a B6 background, were bred on to a Thy1.1-congenic background. Kieper et al., J Exp Med 195: 1533, 2002; Pircher et al., Nature 351: 482, 1991. All these mice, including IL-15^{-/-} mice on a B6 background, were maintained in our animal facility and used at 3-6 months of age. IL-2^{-/-} and CD25^{-/-} mice were bred from heterozygote breeders and screened by standard PCR protocols provided on the website of The Jackson Laboratory. Judge et al., J Exp Med 196: 935, 200. Experiments involving the use of animals were approved by the Institutional Animal Care and Use Committee at TSRI.

Flow Cytometry and Cell Sorting. Suspensions of spleen or pooled (inguinal, axillary, cervical and mesenteric) LN cells were prepared according to standard protocols and stained for FACS® analysis or sorting using PBS containing 1% FCS and 2 mM EDTA with the following mAbs (from BD Biosciences unless otherwise stated): Alexa Fluor 405-conjugated B220 (RA3-6B2, Caltag Laboratories); PerCP-Cy5.5-conjugated CD3 (145-2C11); Alexa Fluor 405-conjugated CD4 (RM4-5, Caltag Laboratories); PerCP-Cy5.5- or APC-Cy7-conjugated CD8α (53-6.7); PE-conjugated CD813 (H35-17.2); FITC- or PE-conjugated CD25 (PC61.5); APC-conjugated CD44 (IM7, eBioscience); APC-conjugated CD90.1 (HIS51, eBioscience); FITC- or PE-conjugated CD122 (TM-β1 or alternatively 5H4); and PE-conjugated Foxp3 (FJK-16s, eBioscience). Intracellular Foxp3 staining was performed following manufacturer's recommendations. In brief, cells were stained for cell surface markers first, then fixed using 2% paraformaldehyde and permeabilized using saponin before intracellular staining Flow cytometry samples were analyzed using a BD LSR II digital flow cytometer. Cell sorting was performed using BD FACS Aria. Purity of the samples was routinely tested after sorting and was over 98%.

T Cell Transfer and Administration of Cytokines and Antibodies In Vivo. FACS®-sorted memory-phenotype (MP) CD44^{hi} CD122^{hi} CD8⁺ T cells (>98% pure) were obtained from spleen or pooled LN of wild-type (WT) B6.PL, IL-7 tg mice on a Thy1.1 congenic background, or CD25^{-/-} mice where indicated; by all parameters tested, MP CD8⁺ cells from IL-7 tg mice were indistinguishable from the (much smaller) population of these cells prepared from normal B6 (or B6.PL) mice. Sorted MP CD8⁺ cells were injected intravenously (iv) at 1-2×10⁶ cells/mouse. rmIL-2 was purchased from eBioscience and stored according to manufacturer's recommendations. The S4B6.1 hybridoma (rat IgG2a) was obtained from the American Type Culture Collection (ATCC) and cultured in vitro under standard conditions (see below) and secreted mAb was obtained from culture supernatant. For comparison, S4B6 IL-2 mAb was also purchased from BD Biosciences. The IL-2 mAbs JES6-1A12 (rat IgG2a) and JES6-5H4 (rat IgG2b) were purchased from eBioscience. F(ab′)₂ preparations of S4B6 IL-2 mAb were custom ordered from BD Biosciences, run on a 10% SDS-polyacrylamide gel under non-reducing conditions to verify digestion, and tested in vitro for their ability to neutralize rmIL-2 (FIG. 12B).

Starting on the day of adoptive cell transfer, age- and gender-matched mice received daily intraperitoneal (ip) injections of PBS, isotype-matched antibody (rat IgG2a or rat IgG2b, respectively), 1.5 μg rmIL-2, S4B6 (50 μg, except for FIG. 2), 50 μg S4B6 plus 10 μg CD122 mAb (TM-β1), 50 μg JES6-1A12, 50 μg JES6-5H4, 1.5 μg rmIL-2 plus S4B6 (50 μg, except for FIG. 8D), 1.5 μg rmIL-2 plus 50 μg JES6-1A12, or 1.5 μg rmIL-2 plus 50 μg JES6-5H4. For the experiments using recombinant human IL-2 (rhIL-2), rhIL-2 and human IL-2 mAb (MAB602, clone 5355) were purchased from R&D Systems. As described above for rmIL-2, mice were injected ip daily with isotype-matched control antibody (mouse IgG2a), 1.5 μg rhIL-2, 50 μg MAB602 hIL-2 mAb or a mixture of 1.5 μg rhIL-2 plus 50 μg MAB602 hIL-2 mAb. For the experiments using rmIL-4, rmIL-4 was purchased from eBioscience and stored according to manufacturer's recommendations. The anti-mouse IL-4 mAb MAB404 (clone 30340, rat IgG1) was obtained from R&D Systems, the second anti-mouse IL-4 mAb 11B.11 (rat IgG1) was provided from the NCI BRB Preclinical Repository (Rockville, Md.). As described above for IL-2, mice were injected ip every other day with isotype-matched control antibody (rat IgG1), 1.5 μg rmIL-4, 50 μg IL-4 mAb or a mixture of 1.5 μg rmIL-4 plus 50 μg IL-4 mAb. 7 days after adoptive cell transfer, spleen and LN cells were analyzed by flow cytometry as described above.

Generation of antigen-specific memory CD8⁺ cells. Thy1.1-marked P14 mice bearing TCR tg CD8⁺ cells, which recognize the lymphocytic choriomeningitis virus (LCMV) gp33-41 epitope, were used as donors. Spleen cells from these mice were treated with complement plus mAbs against heat-stable antigen (J11d), CD4 (RL172), and MHC-II mAb (28-16-8s), as previously described, in order to obtain ˜95% pure CD8⁺ Thy1.1⁺ cells, which were ˜90% Vα2⁺ and thus TCR tg. Kosaka et al., J Exp Med 176: 1291, 1992. These purified cells were then adoptively-transferred iv to B6 (Thy1.2) mice at 5×10⁴ cells/mouse, which received 1 d after cell transfer 2×10⁵ plaque-forming units of the LCMV strain Armstrong ip. Mice were then left for >2 months to allow for generation of CD8⁺ memory cells. At that time, CD8⁺ cells were purified from spleens by complement plus mAbs as described above, or, alternatively, by FACS®-sorting for CD122^{hi} CD44^{hi} CD8⁺ cells. Purified CD8⁺ cells, containing ˜16-20% Thy1.1⁺ Vα2⁺ LCMV-specific memory T cells, were then CFSE labeled and adoptively transferred at 10-15×10⁶ cells/mouse to B6 (Thy1.2) mice, which subsequently received daily ip injections of PBS, 1.5 μg rmIL-2, 50 μg S4B6 IL-2 mAb or 1.5 μg rmIL-2 plus 50 μg IL-2 mAb. 7 days after adoptive cell transfer, spleen and LN cells were analyzed by flow cytometry as described above.

Measurement of Cell Turnover In Vivo. Proliferation of cells in vivo was measured using dilution of the dye CFSE or incorporation of bromodeoxyuridine (BrdU) (0.8 mg/ml) given in the drinking water. Kieper et al., J Exp Med 195: 1533, 2002; Tough et al., J Exp Med 179: 1127, 1994. CFSE staining was performed as follows: cells were resuspended in PBS containing 1% FCS at 10-20×10⁶ cells/ml and stained with 1 μl of 5 mM Vybrant CFDA SE Cell Tracer kit (Molecular Probes) per milliliter of cell suspension for 10 minutes at 37° C., and then washed twice with ice-cold PBS containing 1% FCS. Intracellular staining for BrdU was performed using the FITC BrdU kit from BD Biosciences following the manufacturer's recommendations.

Proliferation In Vitro. MP CD8⁺ cells were sorted by flow cytometry from B6 or CD25^{-/-} mice. For CD3-activated cells, CD8⁺ cells were purified from pooled LN of young B6 mice using the MACS® CD8⁺T cell isolation kit (Miltenyi Biotec); these cells were >95% CD8⁺ and consisted of ˜90% CD44^{lo} naïve cells. Alternatively, cells were purified using FACS® to obtain >99% pure CD44^{lo} CD8⁺ cells. These purified CD8⁺ cells were then activated using plate-bound CD3 mAb (145-2C11, eBioscience). Where indicated, a fixed concentration (10 μg/ml) of isotype-matched control mAb, CD25 mAb (PC61.5, eBioscience), or CD122 mAb (TM-β1, BD Biosciences) was added to the cells prior to mixing them with IL-2/IL-2 mAb complexes. Cells were seeded at 5×10⁴ cells per well in 96-well plates, and titrated concentrations of complexes of rmIL-2 plus isotype-matched control mAb, rmIL-2 plus S4B6 IL-2 mAb, or rmIL-2 plus JES6-1A12 IL-2 mAb were added to the wells. The rmIL-2/IL-2 mAb complexes were at an exact 2:1 molar ratio to avoid excess of either of the two components. Cells were cultured under standard conditions (37° C., 7% CO₂, humidified atmosphere) for 3 days. [³H]-thymidine (1 μCi/ml) was added for the last 16 h, and cell proliferation was assessed measuring [³H]-thymidine incorporation on a liquid scintillation counter (Harvester 96, Tomtec).

Bone Marrow Reconstitution. Bone marrow (BM) cells were obtained from normal B6 mice, and left either untreated or were depleted of T cells using complement plus mAbs against CD4 (RL172) and CD8 (3.168), which eliminated over 95% of the mature T cells in BM. Recipient B6 mice were irradiated at 1000 cGy before iv injection of 5-10×10⁶ unseparated or T cell-depleted BM cells, respectively. Subsequently, daily injections of PBS, 1.5 μg rmIL-2, 8 μg S4B6 IL-2 mAb or 1.5 μg rmIL-2 plus 8 μg S4B6 IL-2 mAb were given ip. 8 days after adoptive transfer spleen cells were stained and analyzed by flow cytometry.

Enzyme-Linked Immunosorbent Assay (ELISA). A standard IL-2 sandwich ELISA was performed according to manufacturer's recommendations using the eBioscience murine IL-2 ELISA kit. In brief, flat-bottom 96-well plates were coated overnight at 4° C. with purified JES6-1 “capture” mAb. The plates were then washed vigorously after which rmIL-2 was added to the wells and incubated for 2 h at room temperature. Subsequently, the plates were washed vigorously, followed by addition of biotinylated JES6-5 “detection” mAb for 1 h at room temperature. Where indicated, titrated concentrations (5-fold dilutions starting at 100 μg/ml) of purified control mAb, purified JES6-1, purified JES6-5, or purified S4B6 IL-2 mAb were added together with the detection mAb. Subsequently, the plates were washed vigorously before adding streptavidin-conjugated horseradish peroxidase for 30 minutes at room temperature. The samples were then developed using the substrate o-phenylenediamine, and, after stopping the reaction with 2 NH₂SO₄, analyzed at 450 nm with an ELISA reader (Spectramax Plus 384, Molecular Devices).

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed is:

1-91. (canceled)

92. An antibody capable of binding a cytokine, or a cytokine and its natural receptor, for use in a method for improving immune function in a mammalian subject, wherein a biological activity of the cytokine is increased when the antibody alone or the antibody together with the cytokine, or the cytokine and its natural receptor, are administered to the subject.

93. The antibody or cytokine and its natural receptor of claim 92, wherein the method for improving immune function comprises:
(a) preventing or treating autoimmune disease;
(b) preventing or treating neoplastic disease;
(c) expanding a hematopoietic cell population; or
(d) preventing or treating infectious disease.

94. The antibody or cytokine and its natural receptor of any one of the preceding claims, wherein the method further comprises increasing presentation of the cytokine to a target cell in the mammalian subject.

95. The antibody or cytokine and its natural receptor of any one of the preceding claims, wherein the method further comprises complexing the antibody with the cytokine or the cytokine with the natural receptor prior to said administration, and administering the cytokine antibody complex or cytokine natural receptor complex to the mammalian subject.

96. The antibody of any one of the preceding claims, wherein the antibody comprises an Fc portion which binds to the cytokine.

97. The antibody of any one of the preceding claims, wherein the cytokine is IL-1, IL-2, IL-3, IL-4, IL-6, IL-7, IL-9, IL-10, IL-12, IL-15, IL-17, IL-21, a type I interferon, or a type II interferon.

98. The antibody of claim 97, wherein the type I interferon is IFNα or IFNβ, and the type II interferon is IFNγ.

99. The antibody of claim 97, wherein the cytokine is interleukin-2 or interleukin-7.

100. The antibody or cytokine and natural receptor of any one of the preceding claims, wherein increasing the biological activity of the cytokine expands a population of T cells, B cells, or NK cells, or a combination thereof.

101. The antibody or cytokine and natural receptor of claim 100, wherein increasing the biological activity of the cytokine expands CD4⁺T regulatory cells.

102. The cytokine and natural receptor of any one of claim 92 to 95 or 100, wherein the cytokine is interleukin-15 and the receptor is interleukin-15 receptor α.

103. The antibody or cytokine and natural receptor of any one of the preceding claims, wherein the autoimmune disease is rheumatoid arthritis, multiple sclerosis, diabetes, inflammatory bowel disease, psoriasis, systemic lupus erythematosus, allergic disease, or asthma.

104. The antibody or cytokine and natural receptor of any one of the preceding claims, wherein the neoplastic disease is cancer, solid tumor, sarcoma, melanoma, carcinoma, leukemia, or lymphoma.

105. The antibody or cytokine and natural receptor of any one of the preceding claims, wherein the method of (c) further comprises providing a therapeutic effect of a cytokine antibody complex to improve hematopoietic cell recovery from hematopoietic cell depletion resulting from irradiation or cytotoxic drug treatment, or primary or secondary immunodeficiency in the mammalian subject.

106. The antibody or cytokine and natural receptor of any one of the preceding claims wherein the method of (d) further comprises administering a vaccine to increase an immune response and to enhance vaccine efficacy.
